(19) United States
(12) Patent Application Publication
(10) Pub. No.: US 2008/0233564 A1
(43) Pub. Date: Sep. 25, 2008

(54) Methods of Using Databases to Greate Gene-Expression Microarrays, Microarrays Greated Thereby, and Uses of the Microarrays

(75) Inventors: Alicia Bertone, Columbus, OH (US); Weisong Gu, Dublin, OH (US)

Correspondence Address: CALFEE HALTER & GRISWOLD, LLP, 800 SUPERIOR AVENUE, SUITE 1400, CLEVELAND, OH, 44114, US

(73) Assignee: THE OHIO STATE UNIVERSITY, Columbus, OH (US)

(21) Appl. No.: 10/597,064

(22) PCT Filed: Jan. 7, 2005
(86) PCT No.: PCT/US2005/000517

§ 371 Date: Nov. 29, 2007

Related U.S. Application Data
(60) Provisional application No. 60/535,111, filed on Jan. 8, 2004.

(51) Int. Cl. C12Q 1/68; C40B 40/06; G06F 7/06; G06F 17/30

(52) U.S. Cl. 435/6; 506/16; 707/3; 707/E17.014

(57) ABSTRACT

Methods of preparing biological databases, and databases prepared according to those methods. In some embodiments, the methods can be performed entirely using computer resources, relying solely on publicly available biological sequence information. The methods of the invention can be used to generate species-specific nucleic acid microarrays

This application claims priority to U.S. Provisional Application No. 60/535,111 filed Jan. 8, 2004, the entire disclosure of which is incorporated herein by reference.

### SEQUENCE LISTING

The instant application contains a “lengthy” Sequence Listing which has been submitted via CD-R in lieu of a printed paper copy, and is hereby incorporated by reference in its entirety. Said CD-R, recorded on Apr. 1, 2005, are labeled “CRF,” “Copy 1,” “Copy 2,” and “Copy 3,” respectively, and each contains only one identical 4.07 MB file (18525413.APP).

### DESCRIPTION OF THE INVENTION

1. Field of the Invention

The present invention is directed to methods of preparing biological databases, and databases prepared according to those methods. In some embodiments, the methods can be performed entirely using computer resources, relying solely on publicly available biological sequence information. The methods of the invention can be used to generate species-specific nucleic acid microarrays.

### BACKGROUND OF THE INVENTION

DNA microarrays are small, solid supports containing thousands of different gene sequences that are immobilized or attached at fixed locations. (Ekins R and Chu F W, “Microarrays: their origins and applications,” Trends Biotechnol 17:217-218 (1999); Lobenhofer E K, Bushel P R, Afshari C A, and Hamadeh H K, “Progress in the Application of DNA Microarrays,” Environ Health Perspect 109(9):881-891 (2001).) This technology has revolutionized the basic approach to research since its invention. Unlike the traditional methods in molecular biology for one gene in one experiment, hundreds to thousands of genes can be analyzed simultaneously under identical conditions to various biological models, including disease, therapy, or experimental manipulation. Microarrays provide unprecedented opportunities for both qualitative and quantitative analysis in gene expression, gene identification and gene alteration detection, such as polymorphisms. (Galamb O, Molnar B, and Tulassay Z, “DNA chips for gene expression analysis and their application in diagnostics,” Orv Hetil 144:21-27 (2003)). The use of larger scale expression profiling permits the classification of genes by biological function, the contribution of patients' disease patterns directly to research, as well as the discovery of genes of unknown function by association with disease. The expression profiles can be diagnostic, prognostic, as well as disease monitoring. (Bubendorf L, “High-throughput microarray technologies: from genomics to clinics,” Eur. Urol 40:231-238 (2001); Crowther D J, “Applications of microarrays in the pharmaceutical industry,” Curr. Opin. Pharmacol 2:551-554 (2002).)

Mammalian commercial DNA microarrays currently exist for human, mouse, cattle, dogs, and rat, but not for the horse or other domestic animals.

There are currently two dominant DNA microarray technologies: spotted microarrays on glass slides, which were first developed at Stanford University (Schena M, Shalon D, Davis R W, and Brown P O, “Quantitative monitoring of gene expression patterns with a complementary DNA microarray,” Science 270:467-470 (1995)), and in situ synthesized oligonucleotide microarrays produced by Affymetrix Inc. Spotted microarrays contain probes that are complementary DNA (cDNA), polymerase chain reaction products or oligonucleotides. Probes are physically deposited on a chemically modified glass slide. Two purified mRNA samples are separately reverse transcribed using two different fluoroprobes and the resulting dye-labeled cDNA populations are used to hybridize on the array under competitive conditions. After hybridization, the array is analyzed with a two-channel fluorescence scanner and the ratio of the two fluorophores can be determined which is later used to reflect the gene expression level of target genes. (Burgess J K, “Gene expression studies using microarrays,” Clin Exp Pharmacol Physiol 28(4):321-328 (2001).) One of the major advantages of cDNA spotted microarrays is that the genetic information need not be known before putting it on the array. Yet if the genetic information is available, oligonucleotides can be specifically designed to uniquely hybridize the target gene.

Here, we describe a unique computer-based approach for the data mining and sequence selection for the equine gene expression microarray from the GenBank database using a series of Java application programs.

### SUMMARY OF THE INVENTION

The present invention is advantageous in providing a new method for obtaining a species-specific collection of nucleic acid sequences from publicly available databases. In particular, the present invention provides: methods of preparing a species-specific nucleic acid database comprising: selecting from a species-non-specific nucleic acid database species-specific nucleic acids comprising coding sequences; selecting from a species-non-specific nucleic acid database species-specific nucleic acids comprising noncoding sequences; selecting from the coding sequences those sequences that are 3′-compete or 3′-coding biased, wherein 3′-coding biased sequences comprise 5′-partial sequences having desirable characteristics; selecting from the noncoding sequences those sequences that include poly-A tails or are derived from sequences that include poly-A tails; reducing redundancy in selected sequences; comparing sequences comprising unannotated sequences to a collection of sequences comprising annotated coding sequences and selecting those sequences satisfying a threshold of similarity; and collecting all selected sequences. In some embodiments, the species-specific nucleic acid database is an equine-specific nucleic acid database. In some embodiments, the species-non-specific nucleic acid database is GenBank.

The present invention also provides arrays comprising a plurality of oligonucleotide probes designed to be complementary to and hybridize under stringent conditions with a gene listed in one of Tables 33, 35, or 37. In some embodiments, the array consists of less than 100 probes that are complementary to genes not listed in Tables 33, 35, or 37.

The present invention also provides arrays comprising a plurality of oligonucleotides, wherein: a) the oligonucleotides are chosen from the nucleic acid sequences shown in Tables 34, 36, or 38, and wherein the array comprises 10 or more of said oligonucleotides; or b) the oligonucleotides comprise nucleotide probes designed to be complementary to, or hybridize under stringent conditions with, 10 or more nucleic acid sequences shown in Tables 34, 36, or 38. In some embodiments, the oligonucleotides comprise nucleotide probes designed to be complementary to, or hybridize under stringent conditions with, 1000, 2000, or 3000 or more nucleic acid sequences shown in Table 34.

The present invention also provides methods for populating a database of species-specific nucleic acid sequences, comprising querying a database of nucleic acid sequences to identify nucleic acid sequences associated with a subject species; processing the identified sequences to create a first subset containing coding sequences and a second subset containing non-coding sequences; dividing the first subset into a plurality of DNA sequences, if present, and a plurality of mRNA sequences; processing the plurality of DNA sequences to derive a plurality of virtual mRNA sequences; dividing the plurality of mRNA sequences into a plurality of complete and mRNA 3′ partial sequences, and a plurality of mRNA 5′ partial sequences; processing the plurality of mRNA 5′ partial sequences to identify a subset of mRNA 5′ partial sequences, each member of the subset satisfying a threshold level of completeness; identifying members of the second subset containing non-coding sequences that correlate with at least one known coding sequence of at least one species other than the subject species; and combining the plurality of virtual mRNA sequences, the plurality of complete and mRNA 3′ partial sequences, the subset of mRNA 5′ partial sequences, and the identified correlated sequences to create the database of species-specific nucleic acid sequences. In some embodiments, the step of identifying includes comparing each member of the second subset to each member of a database containing annotated human nucleic acid sequences. In some embodiments, the step of identifying includes comparing each member of the second subset to each member of a database containing annotated human and mouse nucleic acid sequences. The database containing annotated human and mouse nucleic acid sequences can be derived from the database of nucleic acid sequences. In some embodiments, the method further comprises eliminating duplicates within the database of species-specific nucleic acid sequences. In some embodiments, the method further comprises populating the database of species-specific nucleic acid sequences with selected species-specific virus definitions. In some embodiments, the method further comprises verifying that each of the identified correlated sequences is represented in sense format.

The present invention also provides methods of identifying changes in gene expression with time, by assaying a biological sample with the microarray of the present invention, repeating the assay after a period of time has elapsed, and comparing the results. Also provided are methods of detecting or monitoring a disease chosen from osteoarthritis, joint inflammation, neurological diseases, such as equine protozoal myelitis, developmental orthopedic diseases, laminitis, and the general condition of stress, comprising testing a biological sample on these microarrays for the presence of a genetic marker associated with the disease being tested for.

Also provided are methods of detecting or monitoring an infectious disease chosen from herpesvirus-2 and equine protozoal myelitis caused by sarcocystis neurona or sarcocystis neurospora, comprising testing a biological sample on a microarray of the invention for the presence of a genetic marker associated with the disease being tested for.

Additional aspects and advantages of the invention will be set forth in part in the description that follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

FIG. 1 is a schematic flow chart of the overall design of 3′-biased equine annotated gene and EST sequence selection.

FIG. 2 shows scatter plots of signal intensities for probe sets in an equine gene expression microarray in various replicates of equine synoviocytes cultured with lipopolysaccharide (LPS; 100 ng/mL; A, C, and E) and without LPS (control; B, D, and F). Lines represent 2-fold, 3-fold, 10-fold, and 30-fold change in gene expression, either up (above midpoint) or down (below midpoint). Light gray points represent genes identified as not expressed or marginally expressed in both replicates, intermediate gray points represent genes identified as expressed in 1 replicate but not expressed in the other replicate, and black points represent genes identified as expressed in both replicates. In all replicates, r, >0.99 and p<0.001.

FIG. 3 shows scatter plots of mean signal intensities for probe sets in an equine gene expression microarray in equine synoviocytes cultured with and without LPS. See FIG. 2 description for key.

FIG. 4 shows validation of high-quality RNA extraction. The RNA was extracted and purified using the Trizol protocol. Peaks for 28S and 18S rRNA indicate high quality non-degraded RNA whereas smaller peaks 20-35 indicate the degree of degradation of RNA.

FIG. 5 shows digital photos of representative samples of cartilage suffering from erosion and fibrillation, as compared to normal cartilage.

FIG. 6 shows a dendogram for clustering experiments.

FIG. 7 shows a scatter plot for horses under stress. Expressed genes (black) for each gene comparison among control (X-axis) and stressed (y-axis) arrays (four control and five stressed produce twenty possible control-stressed array comparisons for 3098 genes=61, 960 dots representing signal log ratios for a gene. Intermediate gray dots were marginally expressed. Black dots are comparisons of genes expressed in at least one of the comparative arrays.) Data analyses were based on the Absolute and Comparative analysis of the CHP files produced upon scanning the microarray and performed by Alan Bakaletz, Bioinformatics and Computational Biology Core, Davis Heart & Lung Research Institute at The Ohio State University.

FIG. 8 shows signal intensity scatter plot of laminitis endothelium (y-axis) Vs Control (x-axis). Four fold change lines are in pairs: y=2x and y=1/2x, y-3x and y=1/3x, y=10x and y=1/10x, y=30x and y=1/30x.

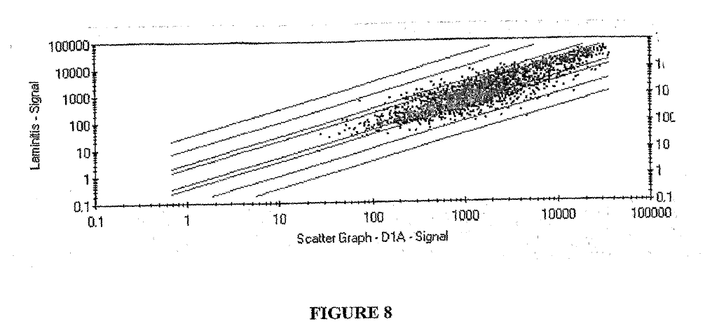

FIG. 9 shows signal intensity scatter plot of Canine OA Vs Control. Four fold change lines are in pairs: y=2x and y=112x, y=3x and y-1/3x, y=10x and y=1/10x, y-30x and y=1/30x.

### DESCRIPTION OF THE EMBODIMENTS

Reference will now be made in detail to specific embodiments of the invention, examples of which are illustrated in the accompanying drawings. Wherever possible, the same reference numbers will be used throughout the drawings to refer to the same or like parts.

The present invention is generally directed to methods for preparing biological databases, and databases prepared according to those methods. The inventive methods can be practiced using readily available hardware and publicly available software. The databases can comprise nucleic acids, including DNA and/or RNA, or polypeptides.

In one embodiment, the invention comprises methods for curating, pruning, and annotating publicly available gene sequences by computer to create high quality nucleic acid sequence data. The data obtained by the present methods can be assembled into a database, which can be used for any purpose, including use in a gene expression microarray.

The methods of the invention take advantage of information available in public databases, including but not limited to, GenBank. As will be readily apparent from this disclosure, other databases can also be used, provided the desired information is available.

The methods of the invention can accommodate selection of any desired characteristics of the nucleic acid sequences. For example, the invention can be used to select all species-specific sequences, such as all equine (Equus caballus), bovine (Bos taurus), ovine (Ovis aries), porcine (Sus scrofa), caprine (Capra hircus), canine (Canis familiaris), feline (Felis catus), avian (domestic chicken, Gallus gallus), or any other desired species. Within any given species, selection can be all inclusive or be made based on tissue, or disease, or pathogen, or any other desired characteristic.

The invention will now be described with reference to a particular embodiment. It should be recognized that the invention comprises other embodiments, and that those of ordinary skill in the art will recognize what those embodiments are. Also, the embodiments described herein comprise several steps or components. It is contemplated that these steps may be rearranged, as desired, to achieve the desired result. The numbering scheme below is simply for clarification in this description and is not intended to define the order of the steps.

Additionally, while the following steps are designed for selecting mRNA sequences, other selections could be made during any step, depending on the desired result. Finally, the following steps selected for 3′-biased mRNA sequences, but other selection forces may be applied, including for example, selecting for all mRNA sequences, selecting for DNA sequences, selecting for complete sequences, etc. The choices will be understood by those of skill in the art upon reading this disclosure.

1. Obtaining a Species-Specific Selection of Nucleic Acid Sequences

In one embodiment of the invention, a species-specific collection of nucleic acid sequences is prepared. In a first step, a public database, such as GenBank, is queried using a species-specific request. For example, to obtain all equine sequences, the database is queried for “Equus caballus,” for bovine, “Bos taurus,” for ovine, “Ovis aires,” for porcine, “Sus scrofa,” for caprine, “Capra hircus,” for canine, “Canis familiaris,” or for feline, “Felis catus.”

It should be recognized that public databases may differ in the information that may be entered for any given field. For example, instead of simply “Equus caballus,” an entry may say “Equus caballus (horse),” or other similar entry. Thus, if desired, care may be taken to use inclusive language in the query to avoid omitting desired entries. Similarly, it should be recognized that entries may refer to a species as a host, such as “Equine lymphoma.” If desired, care can be taken to use exclusive language to avoid including such entries.

2. Separating Coding Sequences (CDS) from Non-Coding Sequences (NonCDS)

The Coding Sequences (CDS) and Non-Coding Sequences (NonCDS) sequences are separated by the program GetCDS. NonCDS can undergo further analysis, as described herein below in step 11. Within the CDS selection, some sequences may comprise DNA and others mRNA.

3. Separation of DNA CDS from mRNA CDS

By the program CheckMRNA, one can separate mRNA sequences from DNA sequences. Sequences identified as “mRNA” are treated further below under step number 7. DNA CDS may further comprise complete and partial sequences.

4. Selection of 3′Complete DNA Sequences

“Complete 3′” DNA coding sequences contain stop codons at the three-prime ends, and thus can be full-length or partial sequences anchored at their three-prime ends. Other sequences are 5′ partial DNA sequences. The DNA CDS from step 3 above can be further selected for “3′ complete” sequences, to remove 5′ partial sequences from the collection. Of course, if desired, partial DNA sequences can be retained and later analyzed and annotated.

5. Removing Duplicate Sequences

Because there is a possibility that multiple entries exist for the same sequence, steps may be taken to remove duplicates. In the case of GenBank sequences, the selected DNA sequences from step 4 can be converted to a uniform format, such as by using the Fasta program, then submitted to an overlap-detecting algorithm, such as the ClusterG program. Any level of scrutiny can be applied in identifying “duplicates.” For example, sequences that are greater than 99%, 98%, 97%, 96%, 95%, 90%, 85%, 80%, 75%, 70%, 65%, 60%, 55%, 50%, or even lower percent, identical can be deemed duplicates and removed. Obviously, a higher level allows for a larger number of similar sequences to be retained, whereas a lower level will have the opposite effect. The desired level can be unique to any situation, and will be determined by the scientist or practitioner using the system, depending on their needs.

6. Identifying “Buried” mRNA Sequences

The non-duplicate DNA CDS can further be examined for the presence of mRNA information. When available, the mRNA information can be collected and further analyzed as described below step number 10.

7. Selection of 3′Complete mRNA Sequences

Like the DNA described above, “3′ Complete” mRNA coding sequences contain stop codons at the three-prime ends, and thus can be full-length or partial sequences anchored at their three-prime ends. Other sequences are 5′ partial mRNA sequences. The mRNA CDS from step 3 above can be further selected for “3′ complete” sequences, to remove 5′ partial sequences from the collection. Unlike with partial DNA sequences, however, partial mRNA sequences are retained for further processing as described in step 9, below.

8. Removing Duplicate Sequences

Because there is a possibility that multiple entries exist for the same sequence, steps may be taken to remove duplicates. In the case of GenBank sequences, the selected complete 3′ mRNA sequences from step 7 above can be converted to a uniform format, such as by using the FastaG program, then submitted to an overlap-detecting algorithm, such as the ClusterG program. Any level of scrutiny can be applied in identifying “duplicates.” For example, sequences that are greater than 99%, 98%, 97%, 96%, 95%, 90%, 85%, 80%, 75%, 70%, 65%, 60%, 55%, 50%, or even lower percent, identical can be deemed duplicates and removed. Obviously, a higher level allows for a larger number of similar sequences to be retained, whereas a lower level will have the opposite effect. The desired level can be unique to any situation, and will be determined by the scientist or practitioner using the system, depending on their needs. Sequences selected are further treated in step 10, below.

9. Annotating Partial mRNA Sequences

Because 5′ partial mRNA from step 7 above may include regions close to the 3′ end, and thus be suitable for use in a microarray, further analysis of these sequences can be performed.

First, the 5′ partial mRNA from step 7 are compared to a combined coding sequence database, such as human+mouse, which can be obtained by querying GenBank for “homo cds” and combining those results with “mus cds.” The coding sequence database can include any sequences, but highly evolved and annotated databases are desirable as the comparative database. The comparison can be achieved using a sequence comparison program such as “BlastN.” The program compares sequences and identifies those that are similar or identical. As with similar programs, the stringency of the comparison can be varied, so as to be more or less selective. Thus, a Blast “score” can be greater than 50, 55, 60, 65, 70, 75, 80, 85, 90, 95, or higher, depending on the desire for identifying similar or identical sequences. Another measurement that can be used is the “E” value, which can be less than 10⁻², 10⁻³, 10⁻⁴, 10⁻⁵, 10⁻⁶, 10⁻⁷, 10⁻⁸, 10⁻⁹, 10⁻¹⁰, or even less, again depending on the desire for identifying similar or identical sequences.

Sequences can then be further selected for their closeness to the 3′ end. “Closeness” is a subjective determination, but can be arbitrarily set at any number of bp, such as less than 1000 bp, 900, 800, 700, 600, 500, 400, 300, 200, 100, or fewer bp, from the 3′ end.

10. Combining and Processing Selected Species Sequences

“Buried” mRNA sequences from step 6, 3′ complete mRNAs from step 8, and selected 5′ partial mRNAs from step 9 are combined, and further processed for duplicates. Again, the sequences can be converted to a uniform format, such as by using the Fasta program, then submitted to an overlap-detecting algorithm, such as the ClusterG program. Any level of scrutiny can be applied in identifying “duplicates.” For example, sequences that are greater than 99%, 98%, 97%, 96%, 95%, 90%, 85%, 80%, 75%, 70%, 65%, 60%, 55%, 50%, or even lower percent, identical can be deemed duplicates and removed. Obviously, a higher level allows for a larger number of similar sequences to be retained, whereas a lower level will have the opposite effect. The desired level can be unique to any situation, and will be determined by the scientist or practitioner using the system, depending on their needs. The selected sequences are further processed as described in step 15, below.

11. Selection of Poly-A ESTs from Non-CDS

Because Non-CDS may still include useful sequences, the Non-CDS from step 2 above can be further processed. The Non-CDS are further selected for those that are identified as including a poly-A tail. This can be performed by querying the GenBank database for a “Yes” or “No” relating to “polyA.” The sequence information from these ESTs may contain the polyA tail if the sequencing process reaches to the 3′ end. However, if the sequencing is initiated at the 5′ end and stops in the middle, the obtained sequence information may not include the polyA tail, although it may be very close to the 3′ end. Therefore, ESTs claiming “PolyA=No” may not necessarily mean that they are not at or close to the 3′ end. Based on this, we first selected the ESTs which claim both “PolyA=Yes” and “PolyA=No” so that a maximal pool of candidate 3′ ESTs could be constructed.

12. Selection of High Quality ESTs

The poly-A-containing ESTs from step 11 above are further processed to select high-quality, vector-trimmed regions. In Genbank there is a feature that states the regions that are of high phred quality with the start and stop positions. All sequences were trimmed to only include these high quality regions based on the start and stop positions. This enhances the confidence that the sequencing was completed accurately.

13. Removing Duplicate Sequences

Again, because there is a possibility that multiple entries exist for the same sequence, steps can be taken to remove duplicates, for example, to maximize the space limitations of a microarray. In the case of GenBank sequences, the selected poly-A ESTs from step 12 above can be converted to a uniform format, such as FastaG format, then submitted to an overlap-detecting algorithm, such as the ClusterG program. Any level of scrutiny can be applied in identifying “duplicates.” For example, sequences that are greater than 99%, 98%, 97%, 96%, 95%, 90%, 85%, 80%, 75%, 70%, 65%, 60%, 55%, 50%, or even lower percent, identical can be deemed duplicates and removed. Obviously, a higher level allows for a larger number of similar sequences to be retained, whereas a lower level will have the opposite effect. The desired level can be unique to any situation, and will be determined by the scientist or practitioner using the system, depending on their needs.

14. Annotating Poly-A EST Sequences

The polyA ESTs can be compared to a combined human+mouse coding sequence database, which can be obtained by querying GenBank for “mus cds” and combining those results with “homo cds.” The comparison can be achieved using a sequence comparison program such as “BlastN.” The program compares sequences and identifies those that are similar or identical. As with similar programs, the stringency of the comparison can be varied, so as to be more or less selective. Thus, a Blast “score” can be greater than 50, 55, 60, 65, 70, 75, 80, 85, 90, 95, or higher, depending on the desire for identifying similar or identical sequences. Another measurement that can be used is the “E” value, which can be less than 10⁻², 10⁻³, 10⁻⁴, 10⁻⁵, 10⁻⁶, 10⁻⁷, 10⁻⁸, 10⁻⁹, 10⁻¹⁰, or even less, again depending on the desire for identifying similar or identical sequences.

Sequences can then be further selected for their closeness to the 3′ end. “Closeness” is a subjective determination, but can be arbitrarily set at any number of bp, such as less than 1000 bp, 900, 800, 700, 600, 500, 400, 300, 200, 100, or fewer bp, from the 3′ end.

Still further, the sense or anti-sense orientation of the sequence can be determined, for example, through use of the BlastN program, which shows the direction of the match. Those sequences deemed to be in anti-sense orientation can be converted to sense sequences by, for example, programs that reverse complement the sequence.

The selected sense-oriented 3′-biased ESTs and converted anti-sense 3′-biased ESTs can be combined together and further processed as described below in step 15.

15. Combining Sequences and Removing Duplicates

The selected sequences from step 10 are combined with those selected from step 14. To reduce the existence of duplicates, further processing can be performed, again to maximize the number of unique sequences represented on a microarray of limited space. The selected sequences can be converted to a uniform format, such as FastaG format, and then submitted to an overlap-detecting algorithm, such as the ClusterG program. Any level of scrutiny can be applied in identifying “duplicates.” For example, sequences that are greater than 99%, 98%, 97%, 96%, 95%, 90%, 85%, 80%, 75%, 70%, 65%, 60%, 55%, 50%, or even lower percent, identical can be deemed duplicates and removed. Obviously, a higher level allows for a larger number of similar sequences to be retained, whereas a lower level will have the opposite effect. The desired level can be unique to any situation, and will be determined by the scientist or practitioner using the system, depending on their needs.

The collection of data created in the steps above can be used for any applicable purpose. Those of skill in the art will recognize uses for such information. The nucleic acid sequences can be used as they are or transformed for any desired use. For example, the sequences can be translated into polypeptide sequences, which can be used for any desired purpose, or probes can be derived from the nucleic acid sequences selected.

Polynucleotide Probes

Probes can be genomic DNA or cDNA or mRNA, or any RNA-like or DNA-like material, such as peptide nucleic acids, branched DNAs and the like. Probes can be sense or antisense polynucleotide probes. Where target polynucleotides are double stranded, the probes may be either sense or antisense strands. Where the target polynucleotides are single stranded, the nucleotide probes are complementary single strands.

Probes can be prepared by a variety of synthetic or enzymatic schemes, examples of which are well known in the art. Probes can be synthesized, in whole or in part, using chemical methods, examples of which are well known in the art (Caruthers et al. (1980) Nucleic Acids Res. Symp. Ser. 215-233). Alternatively, the probes can be generated, in whole or in part, enzymatically.

Nucleotide analogs can be incorporated into polynucleotide probes by methods well known in the art. The incorporated nucleotide analogues should serve to base-pair with target polynucleotide sequences. For example, certain guanine nucleotides can be substituted with hypoxanthine, which base-pairs with cytosine residues. However, these base pairs may be less stable than those between guanine and cytosine. Alternatively, adenine nucleotides can be substituted with 2,6-diaminopurine, which can form stronger base pairs than those between adenine and thymidine. Additionally, polynucleotide probes can include nucleotides that have been derivatized chemically or enzymatically. Typical chemical modifications include derivatization with acyl, alkyl, aryl, or amino groups.

The probes can be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical, and/or chemical means. The labeling moieties include, for example, radioisotopes, such as ³²P, ³³P, or ³⁵S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

Probes can be immobilized on a substrate, examples of which include but are not limited to, rigid and/or semi-rigid supports including membranes, filters, chips, slides, wafers, fibers, magnetic or nonmagnetic beads, gels, tubing, plates, polymers, microparticles, and capillaries. Substrates can have a variety of surface forms, such as wells, trenches, pins, channels and pores, to which the probes are bound. The substrates can be optically transparent.

Hybridization Complexes

Hybridization causes a probe and a complementary target to form a stable duplex. In the case of polynucleotide probes and targets, this occurs through base pairing. Hybridization methods are well known to those skilled in the art (See, e.g., Ausubel (1997; Short Protocols in Molecular Biology, John Wiley & Sons, New York N.Y., units 2.8-2.11, 3.18-3.19 and 4.6-4.9). Conditions can be selected for hybridization where exactly complementary target and polynucleotide probe can hybridize, i.e., each base pair must interact with its complementary base pair. Alternatively, conditions can be selected where target and polynucleotide probes have mismatches but are still able to hybridize. Suitable conditions can be selected, for example, by varying the concentrations of salt in the prehybridization, hybridization, and wash solutions, or by varying the hybridization and wash temperatures. With some membranes, the temperature can be decreased by adding formamide to the prehybridization and hybridization solutions.

Hybridization conditions are based on the melting temperature (Tₘ) of the nucleic acid binding complex or probe, as described in Berger and Kimmel (1987) Guide to Molecular Cloning Techniques, Methods in Enzymology, Vol. 152, Academic Press. The term “stringent conditions,” as used herein, is the “stringency” which occurs within a range from about Tₘ-5 (5° below the melting temperature of the probe) to about 20° C. below Tₘ. As used herein, “highly stringent” conditions employ at least 0.2×SSC buffer and at least 65° C. As recognized in the art, stringency conditions can be attained by varying a number of factors, including for example, the length and nature, i.e., DNA or RNA, of the probe; the length and nature of the target sequence; and the concentration of the salts and other components, such as formamide, dextran sulfate, and polyethylene glycol, of the hybridization solution. All of these factors can be varied to generate conditions of stringency which are equivalent to the conditions listed above.

Hybridization can be performed at low stringency with buffers, such as 6×SSPE with 0.005% Triton X-100 at 37° C., which permits hybridization between target and polynucleotide probes that contain some mismatches to form target polynucleotide/probe complexes. Subsequent washes can be performed at higher stringency with buffers, such as 0.5×SSPE with 0.005% Triton X-100 at 50° C., to retain hybridization of only those target/probe complexes that contain exactly complementary sequences. Alternatively, hybridization can be performed with buffers, such as 5×SSC/0.2% SDS at 60° C. and washes are performed in 2×SSC/0.2% SDS and then in 0.1×SSC. Background signals can be reduced by the use of detergent, such as sodium dodecyl sulfate, Sarcosyl, or Triton X-100, or a blocking agent, such as salmon sperm DNA.

Other procedures for the use of microarrays are available in the art, and are provided, for example, by Affymetrix. In this regard, reference is made to the Affymetrix GeneChip® Expression Analysis Technical Manual, the entire disclosure of which is incorporated herein by reference.

Microarray Construction

The nucleic acid sequences can be used in the construction of microarrays. Methods for construction of microarrays, and the use of such microarrays, are known in the art, examples of which can be found in U.S. Pat. Nos. 5,445,934, 5,744,305, 5,700,637, and 5,945,334, the entire disclosure of each of which is hereby incorporated by reference. Microarrays can be arrays of nucleic acid probes, arrays of peptide or oligopeptide probes, or arrays of chimeric probes—peptide nucleic acid (PNA) probes. Those of skill in the art will recognize the uses of the collected information.

One particular example, the in situ synthesized oligonucleotide Affymetrix GeneChip system, is widely used in many research applications with rigorous quality control standards. (Rouse R. and Hardiman G. Pharmacogenomics 5:623-632 (2003).). Currently the Affymetrix GeneChip uses eleven 25-oligomer probe pair sets containing both a perfect match and a single nucleotide mismatch for each gene sequence to be identified on the array. Using a light-directed chemical synthesis process (photolithography technology), highly dense glass oligo probe array sets (>1,000,000 25-oligomer probes) can be constructed in a ˜3×3-cm plastic cartridge that serves as the hybridization chamber. The ribonucleic acid to be hybridized is isolated, amplified, fragmented, labeled with a fluorescent reporter group, and stained with fluorescent dye after incubation. Light is emitted from the fluorescent reporter group only when it is bound to the probe. The intensity of the light emitted from the perfect match oligoprobe, as compared to the single base pair mismatched oligoprobe, is detected in a scanner, which in turn is analyzed by bioinformatics software (http://www.affymetrix.com). The GeneChip system provides a standard platform for array fabrication and data analysis which permits data comparisons among different experiments and laboratories.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute detailed modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

Further details of the invention can be found in the following examples, which further define the scope of the invention.

### Example 1

### Construction of Equine Nucleic Acid Database and Microarray

All gene sequences were obtained from the public (GenBank) database, which is maintained at the National Center for Biotechnology Information (NCBI). The sequences were obtained by queries to the GenBank and the returned results were downloaded in GenBank format to the local computer.

The project was completed by using a series of Java application programs which were run under the JAVA™ 2 Runtime Environment, Standard Edition, Version 1.4.1 from the Sun Microsystems, Inc. using a Dell Optiplex GX240 Intel(R) Pentium (R) 4 CPU 1.70 GHz with 256 MB of RAM with Microsoft Windows XP Professional Version 2002 operating system. The BlastN and BlastX were conducted using the bioinformatics resources at the Ohio Supercomputer Center (http://www.osc.edu). Table 1 lists all the programs used.

TABLE1
Software and programs used

| Name | Function |
|---|---|
| GetEquine | Selects the gene sequences which |
| | are from the source of either equus |
| | caballus or equus caballus (horse) |
| CheckCDS | Collects the coding sequences and |
| | non-coding sequences separately |
| GetThreePrimeCompleteCDS | Selects the coding sequences which |
| | contain the stop codons at the 3′ |
| | ends. |
| CheckMRNA | Splits the gene sequences into mRNA |
| | sequences and DNA sequences |
| FastaG | Transforms the gene sequences in |
| | GenBank format to FASTA format |
| ClusterG | Identifies the unigene sets; if |
| | sequences are found >90% identical |
| | match, only the longest sequence is |
| | stored |
| FastaCombine | Combines different FASTA files to one |
| | FAST file |
| GetPolyAEST | Selects ESTs which claim as |
| | “PolyA = Yes” or “PolyA = No” |
| SelectHighQualityEST | Selects the high phred quality region |
| | of the ESTs based on the annotated |
| | start and stop positions in the |
| | GenBank format |
| GetRC | Obtains the reverse complementary |
| | sequence of a target sequence |
| BlastN | Nucleotide-nucleotide sequence |
| | comparison |
| BlastX | Nucleotide-protein sequence |
| | comparison |

The source code for each program is provided in Appendix A.

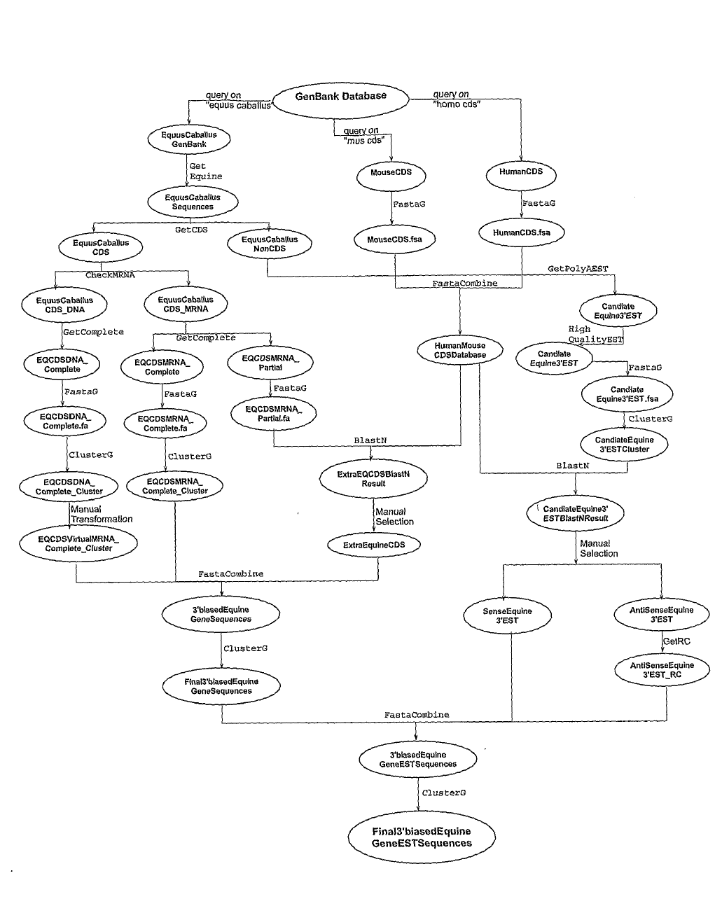

The overall design steps in selecting the 3′ equine annotated genes and ESTs are summarized in FIG. 1.

Construction of Equine, and Human/Mouse Sequence Databases

Equine gene sequences were first obtained through a query of “equus caballus” to the GenBank database at the NCBI web site. A total of 20,022 sequences were returned (as of June 2003) and downloaded in GenBank format to the local computer. Program GetEquine was performed to specifically select those gene sequences that are from either equus caballus or equus caballus (horse), and 18,924 sequences were obtained and named as “EquusCaballusSequences.” This is the original database from which 3′ equine coding sequences and 3′ equine ESTs were identified.

By a query of “homo cds” to the GenBank database at the NCBI web site, 208,480 human sequences (as of the date the Genbank was accessed) were returned and downloaded in GenBank format to the local computer, which were then transformed to FASTA format using the FastaG program. Similarly, by a query of “mus cds,” 205,373 mouse sequences (as of the date the GenBank was accessed) were obtained and stored in FASTA format. The resulting human and mouse coding sequences were combined and a correspondent HumanMouseCDS database was created at the Time Logic DeCypher System at the Ohio Supercomputer Center (http://www.osc.edu).

Selection of 3 Equine Coding Sequences

To screen out the 3′ equine cDNA sequences, program CheckCDS was first applied to the EquusCaballusSequences, with 981 equine coding sequences and 17,943 equine non-coding sequences identified, respectively. The equine coding sequences contain both mRNA and DNA sequences. DNA sequences contain alternative exons and introns, and the latter are removed to produce the mature mRNA. Preferably, mRNA sequences are selected for a gene expression microarray. Program CheckMRNA was performed on the EquusCaballusCDS file, with 436 equine mRNA coding sequences and 545 equine DNA coding sequences identified, respectively.

The equine mRNA coding sequences were further split into two-hundred 5′ partial coding sequences and two-hundred thirty-six 3′ complete coding sequences using the GetThreePrimeCompleteCDS program. 3′ complete coding sequences contain stop codons at the three-prime ends, and hence are either full-length sequences or partial sequences yet 3′ anchored. All these two-hundred thirty-six 3′-anchored sequences were collected for further analysis. Similarly, the equine DNA coding sequences were split into one-hundred thirty-eight 3′ complete coding sequences and four-hundred seven 5′ partial coding sequences. Only the 3′ complete DNA sequences were subjected to further analysis, but 5′ DNA partial sequences could be further evaluated if desired. (See Table 2.)

It is quite often that one single gene may be represented by several sequences, each with a different GenBank Accession Number. The same genes may be sequenced and deposited separately by different labs, or the gene sequences may first be deposited into GenBank as partial coding sequences and later as complete sequences. Therefore, multiple sequences, although with different GenBank Accession Numbers, can actually represent the same gene.

To address this potential problem, the FastaG program was first applied to transform the sequences from the GenBank format to the FASTA format, in which the sequence begins with a single-line description followed by lines of sequence data. Then the ClusterG program was used to identify the unigene clusters and only keep the longest sequence for each cluster. One-hundred ninety-five equine mRNA 3′ complete coding sequence clusters and fifty equine DNA 3′ complete coding sequence clusters were obtained. Because the complete gene (DNA) sequences may contain introns, the virtual respective mRNA sequences of the above equine DNA sequences were obtained by selecting the mRNA or CDS features at the respective GenBank website. The equine mRNA and virtual mRNA sequences were combined with the FastaCombine program and screened again with the ClusterG program for unigene clusters and the final 209 equine annotated 3′ coding sequences were identified. These equine sequences are either full-length sequences or 3′ anchored.

This screening was based on selecting the 3′-biased coding sequences. However, some partial sequences may actually contain regions close to the 3′ end and thus could also be suitable for use in a microarray. To capture these sequences, the two-hundred 5′ partial equine mRNA coding sequences were first reduced to 149 clusters with the ClusterG program. Sequence comparisons of these clusters were performed against the HumanMouseCDS database using the BlastN program at the Time Logic DeCypher System at Ohio SuperComputer Center. The blast result was manually examined and a total of 83 equine partial coding sequences which are in close proximity (i.e., within 500 bp) to the 3′ end or important to our research were identified and combined with the previously identified 209 3′ equine coding sequences using the FastaCombine program. Program ClusterG was performed on the combined sequences and 290 final equine annotated gene sequences were ultimately selected for the microarray. Table 2 summarizes the result in each step of selecting the 3′ equine coding sequences.

TABLE 2
Results for analyses of a public database to identify
3′ equine coding sequences

| Sequence | Number |
|---|---|
| Equine sequences | 18,924 |
| Equine coding sequences | 981 |
| Equine coding sequences, mRNA | 436 |
| Equine coding sequences, mRNA, 3′ complete | 236 |
| Equine coding sequences, mRNA, 3′ complete cluster | 195 |
| Equine coding sequences, mRNA, partial | 200 |
| Equine coding sequences, partial mRNA selected | 83 |
| Equine coding sequences, DNA | 545 |
| Equine coding sequences, DNA, 3′ complete | 138 |
| Equine coding sequences, DNA, 3′ complete cluster | 50 |
| Equine coding sequences, selected mRNA and DNA | 328 |
| Equine coding sequences selected | 290 |

The selected annotated equine gene sequences were also subjected to the BlastX assay against the SwissProt database (Gasteiger E. et al. Curr Issues Mol Biol 3(3):47-55 (2001)) to confirm the sequence orientation, and all sequences were shown in the sense orientation (data not shown).

Selection of 3′ Equine ESTs

The 3′ equine ESTs were isolated from the 17,943 equine non-coding sequences. Candidate 3′ equine ESTs were first obtained using the GetPolyAEST program against the EquusCaballusSequences. Program GetPolyAEST selects the EST sequences which indicate as “PolyA=Yes” or “PolyA=No”. As noted above, the sequence information from these ESTs may contain the polyA tail if the sequencing process reaches to the 3′ end. However, if the sequencing is initiated at the 5′ end and stops in the middle, the obtained sequence information may not include the polyA tail, although it may be very close to the 3′ end. Therefore, ESTs claiming “PolyA=No” may not necessarily mean that they are not at or close to the 3′ end. Based on this, we first selected the ESTs which claim both “PolyA=Yes” and “PolyA=No” so that a maximal pool of candidate 3′ ESTs could be constructed. A total of 8,752 putative equine 3′ ESTs were obtained. Then the SelectHighQualityEST program was applied to specifically select the high-quality, vector-trimmed regions and transform into FASTA format.

The resulting high quality ESTs (8,752 sequences) were subjected to the ClusterG program to obtain EST clusters (4,139 clusters). Table 3 shows the 3′ ESTs. (We selected the longest sequence for each cluster. Longer sequences can be obtained by sequence assembly. For long sequences, the whole sequence is fragmented and each fragment is sequenced individually and the whole sequence is obtained by assembly later. Some sequencing may be performed in both directions. Through assembly, more complete sequences can be obtained, if there is enough overlap exists between the fragments.)

TABLE 3
Results of analyses to identify equine 3′ sequences
for use in a gene expression microarray

| Sequence | Number |
|---|---|
| Equine sequences | 18,924 |
| Equine EST with polyA* | 8,752 |
| Equine polyA EST cluster | 4,139 |
| Equine EST cluster with algorithm confirmation | 3,791 |
| Equine EST screened | 3,155 |
| Sense EST | 2,856 |
| Antisense EST | 299 |
| Equine coding sequences selected | 290 |
| Equine 3′ sequences | 3,288 |
| Final equine sequences selected for the microarray | 3,098 |

*Equine sequences with and without the polyadenylation A (polyA) sequence.
EST = Established sequence tag.

To obtain the annotations and 3′ bias confirmation, the equine ESTs were blasted against the HumanMouseCDS database using the BlastN algorithm at the Ohio SuperComputer Center facility. A total of 3,791 equine EST clusters had blast hits with a Blast score >60. Of these, only sequences with blastE values of <10⁻⁸ were considered candidates for selection. (Makabe et al. Development 128:2555-2567 (2001)). The blast result also was examined manually to remove any ESTs that matched to the 5′ end of the corresponding human or mouse coding sequences. A total of 3,155 ESTs were identified as 3′ biased. The orientations of the ESTs were also derived from the blast results by inspection of the direction of the sequence match (blast hit), with 2,856 in sense orientation and 299 in antisense orientation (Table 3). The reverse complementary sequences of the antisense ESTs were obtained by the program GetRC and were combined with the sense equine ESTs. The resulting ESTs were also combined with the annotated equine coding sequences and undergone the cluster analysis again. A total of 3,288 equine 3′ coding sequences and 3′ ESTs were initially selected, from which 191 were omitted because the possible probe set was of low quality, leaving 3,098 equine coding sequences for the equine gene expression microarray. (In fact, 3,099 equine origin gene sequences were idenfied, but the first, GBEQ0001 is Equus caballus partial 18S rRNA, which was added as a reference gene.)

Note that many of the annotated genes that were publicly available were from laboratories studying musculoskeletal conditions. In total, this may include 100-200 genes. Thus, in the end, the collection of sequences had a slight bias toward musculoskeletal genes.

A complete list of the sequences are listed in attached Tables. Table 39 shows the GB . . . identification codes for the sequences included on the microarray. Table 33 identifies the GenBank accession numbers for all 3,289 equine sequences initially selected (from which the 3,098 were ultimately chosen); Table 34 shows the equine sequences (SEQ ID NOS 1-3289) corresponding to Table 33.

Preparation of the Microarray

The probe set design was accomplished based on the selected equine sequences according to Affymetrix's chip design guide. The probe sets were selected by the following parameters: probe set score, gap multiplier, cross hybridization multiplier, probe count, raw standard deviation, siflength, etc. Each sequence was checked for unique, identical, or mixed probe sets. Probe sets with a score no less than 2.0 for unique set or a score no less than 4.0 for identical or mixed set were selected. A total of 68,266 equine oligonucleotide probes were included on a high density microarray, with average 11 perfect matches and 11 single nucleotide mismatches for each equine gene.

2. Discussion

Genetic information has been exploding dramatically since its construction. At the time of the equine microarray design, over 20,000 equine sequences were available in the public database (GenBank). How to data-mine the 3′-biased sequences is an issue in generating gene expression microarrays, including equine microarrays. Here, we have disclosed a unique computer-based approach that is applicable for creating gene expression microarrays for any other species. The approach generally involves two major steps: identifying the 3′ coding sequences and 3′ ESTs.

In identifying the 3′ equine coding sequences, we first focused on the selection of full-length coding sequences and partial sequences with 3′ end. This is done by selecting the coding sequences with the stop codon at the 3′ end. This approach ensures that sequences selected are 3′ anchored. Some of them also contain the 3′-untranslated regions, which may be more species-specific compared to the coding region. To capture additional coding sequences for the microarray, we performed the blast analysis for the partial coding sequences against the self-constructed HumanMouseCDS database instead of the non-redundant (nr) nucleotide database available at NCBI. The HumanMouseCDS database is actually a subset of the nr database. Most of the sequences are annotated human or mouse coding sequences. Therefore, the blast result based on this database provides more useful information, which was especially valuable in the equine EST annotation and sequence orientation determination. Moreover, as the HumanMouseCDS database is much smaller, the computing time for the blast assay is tremendously decreased.

One approach in constructing the cDNA library used for transcript sequencing is using the oligo-dT as the primer in the first strand cDNA synthesis. This would preferentially begin sequencing from the 3′ end due to priming on the polyA tail. In other methods, the sequence information from these ESTs may contain the polyA tail if the sequencing process reaches to the 3′ end. However, if the sequencing is initiated at the 5′ end and stops in the middle, the obtained sequence information may not include the polyA tail, although it may be very close to the 3′ end. Therefore, ESTs claiming “PolyA=No” may not necessarily mean that they are not at or close to the 3′ end. Based on this, we first selected the ESTs which claim both “PolyA=Yes” and “PolyA=No” so that a maximal pool of candidate 3 ESTs could be constructed.

ESTs are short sequences, representing only fragments of genes, not complete coding sequences. The sequences may be in either sense or antisense orientation. Therefore, a major effort and emphasis is focused on how to best annotate these ESTs. In fact, we first annotated the equine ESTs with blast analysis against the nr database (data not shown). However, an overwhelming number of hits occurred between the ESTs and sequences without much useful information, as the hits occurred with the chromosomal sequences, cDNA clones, etc. Therefore, we modified the blast analysis against the self-constructed HumanMouseCDS database that contained more concentrated annotated human and mouse coding sequences. Approximately 92% of the ESTs had blast hits and putative annotations were provided. (Annotations were categorized based on the published papers. Escribano J. and Coca-Prados, M., Molecular Vision 8:315-332 (2002); Lo J. et al. Genome Research 13(3):455-466 (2003)). (See Table 4.)

For the gene expression microarray, further probe design could be based on the antisense strand of the selected sequences. The array can be either cDNA spotted microarray (the clones can be purchased or self obtained by PCR) or the Affymetrix oligonucleotide GeneChip. cDNA spotted microarrays use longer sequences as probes which are advantageous in that sequences could be spotted first without being known and the gene sequence of interest could be determined later. However, this approach is labor intensive and costly in producing and maintaining the clones or PCR products. Errors may occur in mis-assigning the clones. (Halgren R G, et al. Nucleic Acids Research 29:582-588 (2001).)

It is difficult to distinguish closely related gene families using cDNA microarray. Also, for rarely expressed genes, it is hard to obtain the suitable cDNA clones. On the other hand, if the sequence information is available, oligonucleotides can be synthesized to hybridize specifically and uniquely to any available target genes. This approach avoids the need to manipulate large cDNA clone libraries. The cross-hybridization problem due to the short length of the probe could be ameliorated by the usage of several probe sets per gene. In the Affymetrix GeneChip system, the use of perfect match and mismatch design provides a control for background noise and cross-hybridization from unrelated targets. The chip cost has now decreased several-fold and become more affordable to academics, compared to large-scale cDNA microarrays.

This is the first published microarray accumulation of equine annotated genes and ESTs and all that is publicly available to date. The equine chip includes equine gene sequences functioning in apoptosis, cell cycle, signal transduction, developmental biology, etc, as listed in Table 4. (Escribano J. and Coca-Prados, M., Molecular Vision 8:315-332 (2002); Lo J. et al. Genome Research 13(3):455-466 (2003)).

Note that the final “annotation” of the equine gene sequences selected was simply a Blast search against the combined mouse and human sequence database, as described above. For the sake of brevity, those results are not shown herein, but can easily be repeated by identifying the GenBank accession number corresponding to the “GB . . . ” identification number, and performing a Blast analysis. (The correlation between the GenBank accession numbers and the GB . . . identification numbers is found in Table 33 for the equine sequences.)

TABLE 4
Characterization of selected equine 3′ coding sequences and 3′ ESTs

| | 3′ coding | |
|---|---|---|
| Protein category | sequence | 3′ EST |
| Enzyme | | |
| Dehydrogenase | 4 | 35 |
| Isomerase | 0 | 15 |
| Kinase | 1 | 78 |
| Phosphatase | 0 | 39 |
| Synthase | 5 | 35 |
| Transferase | 1 | 37 |
| Oxidase | 0 | 8 |
| Peptidase | 0 | 6 |
| Others | 14 | 69 |
| Protein synthesis | | |
| Ribosomal protein | 5 | 105 |
| Initiation, elongation, and other factors | 0 | 49 |
| RNA binding | 2 | 108 |
| DNA binding | 5 | 203 |
| Transcription factor | 3 | 64 |
| Protein degradation | 8 | 62 |
| Membrane protein | 2 | 53 |
| Cellular signaling | | |
| Receptor and receptor-related | 32 | 223 |
| Ligand and other exchange factors | 62 | 142 |
| Structural protein | 21 | 98 |
| Cell division | 5 | 41 |
| Cell adhesion | 2 | 12 |
| Cell differentiation | 2 | 15 |
| Ligand binding or carrier | 5 | 102 |
| Transporter | 8 | 74 |
| Antioxidant | 1 | 6 |
| Immune-related proteins | 33 | 152 |
| Lipoprotein | 1 | 3 |
| Apoptosis | 1 | 24 |
| Chaperone | 3 | 21 |
| Enzyme inhibitor | 7 | 33 |
| Enzyme activator | 0 | 10 |
| Developmental protein | 4 | 27 |
| Motor | 0 | 10 |
| Unclassified | 53 | 849 |

Data from this microarray will provide insight into gene expression for equine specific diseases and conditions. Thousands of equine ESTs whose genetic functions were unknown previously are now annotated. This not only enriches the equine gene expression profile, but also will provide a solid base for future full-length gene discovery and analysis.

### Example 2

### Equine Gene Expression and Performance of the Microarray

Materials and Methods

Equine synoviocytes were obtained from adult horses and cultured in monolayer in Dulbecco modified Eagle's medium (DMEM, Gibco, Grand Island, N.Y.) that contained glutamine supplemented with 10% fetal bovine serum, 100 U of penicillin/mL, and 100 μg of streptomycin/mL. Cultures were maintained in a humidified atmosphere containing 5% carbon dioxide at 37° C. Lipopolysaccharide from Escherichia coli 055:B5 (LPS from Escherichia coli 055:B5, Sigma Chemical Co, St Louis, Mo.) at concentrations of 0 and 100 ng/mL was added, and cells were culture for 2.5 hours. Total RNA was isolated by use of a commercial protocol (RNeasy Mini protocol, Qiagen, Valencia, Calif.) for total RNA isolation from animal cells. The RNA samples were separated and developed by use of 1% agarose gel electrophoresis, and sample concentration and purity were measured by use of UV spectra (260 and 280 nm).

All protocols were conducted in according with the manufacturer's instructions (Affymetrix. Affymetrix GeneChip expression analysis technical manual. Santa Clara, Calif.: Affymetrix, 2003). Total RNA (5 μg) was reverse transcribed into double-stranded cDNA by use of a polymerase (Superscript II, Invitrogen, Carlsbad, Calif.) and the T7-(dT) 24 primer (T7-(dT) 24 primer, Qiagen, Valencia, Calif.). Biotinylated cRNA was synthesized by in vitro transcription. The cRNA products were fragmented prior to hybridization overnight at 45° C. for 16 hours. Microarrays were washed at low- and high-stringent conditions and stained with streptavidin-phycoerythrin in accordance with an established protocol (EukGE-WS2, Affymetrix, Inc., Santa Clara, Calif.).

Data analysis was performed by use of commercially available software packages (Microarray suite 5.0, Affymetrix Inc, Santa Clara, Calif.; MicroDB, Affymetrix Inc, Santa Clara, Calif.; Data Mining Tool 3.0, Affymetrix Inc, Santa Clara, Calif.). To test their performance, microarrays were probed in triplicate with the same fragmented cRNA samples from normal equine synoviocytes and LPS-challenge exposed equine synoviocytes. Variables for performance of the microarray, such as signal intensity, were determined by use of statistical algorithms.

Results

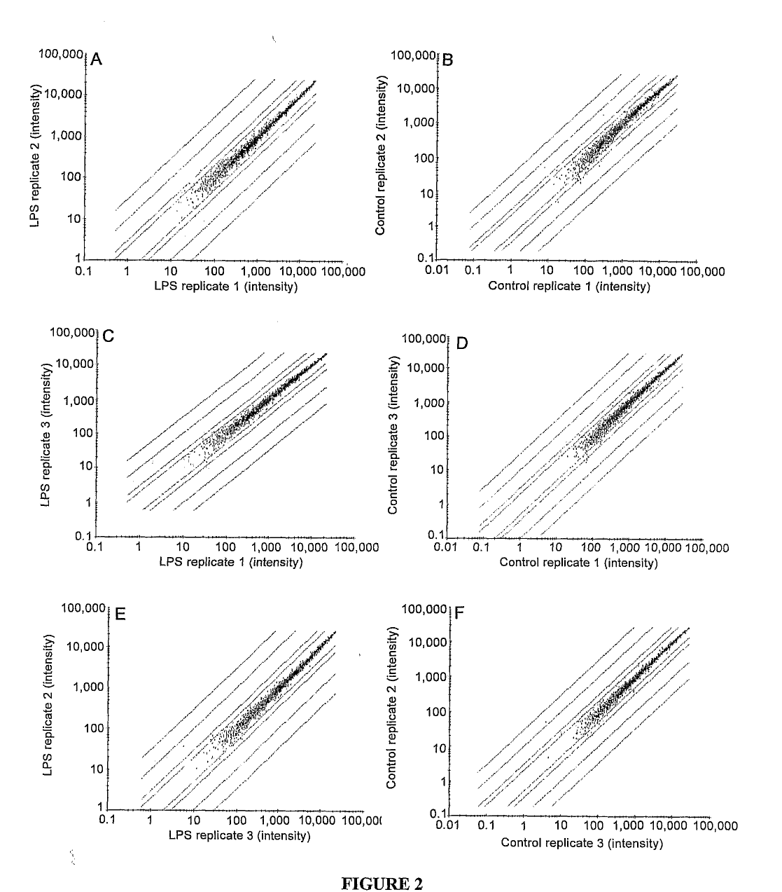

In total, two thirds of the sequences represented on the array were expressed in equine synoviocytes (LPS-treated and control synoviocytes). For each condition, replicates were highly correlated (FIG. 2). Correlation was the highest in expressed genes but was less in nonexpressed or marginally expressed genes. Regardless, there was a high overall correlation (r, >0.99) among replicates. Mean signal intensity was low for the nonexpressed genes (<87) and ranged from 176 to 226 for marginally expressed genes. Gene expression in these categories (nonexpressed or marginally expressed) was low relative to the mean signal intensity of expressed genes (range 2,576 to 2,684; Table 5).

TABLE 5
Results of the equine gene expression microarray for equine
synoviocytes cultured with the addition of lipopolysaccharide (LPS; 100 ng/mL)
and without LPS (control cells)

| | | | Expressed | | Not expressed | | Marginal | |
|---|---|---|---|---|---|---|---|---|
| | Detection of genes | | Mean | | Mean | | Mean | |
| | Signal | Intensity | signal | Genes | signal | Genes | signal | Genes |
| Synoviocytes | Mean | Maximum | intensity | No. (%) | intensity | No. (%) | intensity | No. (%) |
| LPS | 1,774 | 22,917 | 2,576 | 2,142 (68) | 87 | 982 (31) | 176 | 38 (1) |
| Control | 1,806 | 27,509 | 2,684 | 2,092 (66) | 85 | 1,029 (33) | 226 | 41 (1) |

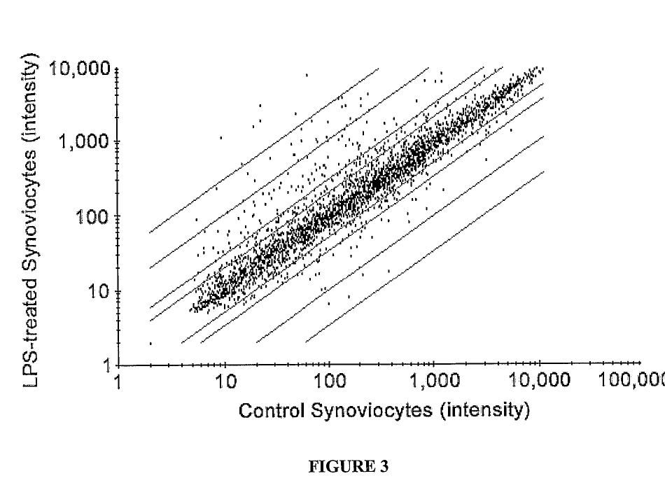

Data from triplicate replicates of each condition (LPS-treated or control synoviocytes) were used to calculate the mean value. Scatter plots of the mean intensity signals of the LPS-treated and control synoviocytes were created (FIG. 3). Although the total number of genes expressed was similar for both conditions, 752 genes were up-regulated and 877 were down-regulated in response to LPS. Among them, several genes had at least a 5-fold change in expression (84 genes were increased and 18 genes were decreased; Table 6). These data were used to create an expression pattern for LPS stimulation of synoviocytes that consisted of 102 genes.

TABLE 6
Genes differentially regulated (>5-fold change) in response to
addition of LPS to cultures of equine synoviocytes

| | GenBank | | GenBank |
|---|---|---|---|
| | Accession | | Accession |
| | number of | | number of |
| | Equine | | Blast |
| Change | Sequence | Full or Provisional annotation | Annotation |
| 164.24 | CD536631 | GRO2 oncogene | XM_003510 |
| 136.67 | CD469327 | Tumor necrosis factor, α-induced protein 6 | NM_007115 |
| 130.48 | AF053497 | Equine melanoma growth-stimulatory activity | AF053497 |
| | | homolog | |
| 108.17 | CD468799 | GRO3 oncogene | XM_031287 |
| 106.81 | AF148882 | Equine matrix metalloproteinase 1 precursor | AF148882 |
| 52.83 | BI960809 | Tumor necrosis factor-stimulated gene 6 | AJ421518 |
| | | protein | |
| 47.09 | CD464860 | Pentaxin-related gene | BC039733 |
| 30.46 | CD535167 | Nuclear factor of κ light polypeptide gene | BC004983 |
| | | enhancer | |
| 29.64 | BM734883 | Chemokine (C-C motif) ligand 7 | NM_006273 |
| 28.71 | BI961093 | Unknown | NM_025079 |
| 28.55 | BM735056 | Interferon regulatory factor 1 | XM_034862 |
| 28.55 | BI961535 | Interleukin-8 | XM_170504 |
| 27.82 | CD469032 | Phosphodiesterase 7A | XM_037534 |
| 25.35 | AY040203 | Equine granulocyte-macrophage colony- | AY040203 |
| | | stimulating factor | |
| 22.51 | BM780597 | CCAAT-enhancer binding protein | XM_171180 |
| 21.73 | CD536763 | Baculoviral IAP repeat-containing 3 | XM_040715 |
| 20.73 | CD468301 | Nuclear factor of κ light chain gene enhancer | BC046754 |
| 19.8 | CD528418 | Prostaglandin endoperoxide synthase-2 | D28235 |
| 19.11 | CD466440 | PP2135 mRNA | AF193048 |
| 18.89 | BI961945 | Unknown | XM_040715 |
| 18.27 | CD468265 | Interleukin-8 | XM_170504 |
| 18.01 | BI961101 | Chemokine (C-C motif) ligand 7 | NM_006273 |
| 15.46 | CD535316 | Interleukin-8 | XM_170504 |
| 15 | CD528575 | Amyloid beta (A4) precursor protein-binding, | NM_019043 |
| | | family B | |
| 14.56 | M27462 | Equine chorionic gonadotropin α-subunit | M27462 |
| 14.21 | CD464433 | Embigin | XM_170912 |
| 14.21 | BM781439 | Chimerin | NM_001822 |
| 13.28 | BI961389 | KIAA0882 protein | XM_093895 |
| 13.05 | AJ319906 | Equine fibroblast growth factor 2 | AJ319906 |
| 12.7 | BM735054 | FAM14A | NM_032036 |
| 11.99 | BM734850 | Uubiquitin-like protein ISG15 mRNA | AY168648 |
| 11.57 | BM734511 | PrP gene | X83416 |
| 10.96 | BM735123 | Hypothetical protein FLJ23231 | NM_025079 |
| 10.85 | AF027335 | Equine prostaglandin G/H synthase-2 gene | AF027335 |
| 10.79 | CD536086 | Tumor necrosis factor, α-induced protein 3 | AA661080 |
| 10.51 | CD466465 | Interleukin-1, α | NM_000575 |
| 10.45 | BM781319 | Cyclin D2 | NM_001759 |
| 10.44 | AF027335 | Equine prostaglandin G/H synthase-2 gene | AF027335 |
| 10.43 | BI960863 | Tumor necrosis factor, α-induced protein 6 | NM_007115 |
| 10.3 | BM735029 | Interferon-induced transmembrane protein 1 | BC000897 |
| 10.18 | CD464478 | Similar to embigin | XM_059649 |
| 10.17 | AF203913 | Equine steroidogenic factor 2 | AF203913 |
| 10.16 | CD536074 | Interferon regulatory factor 1 | XM_034862 |
| 10.02 | CD468537 | Unknown | CD468537 |
| 9.02 | AF038127 | Equine dermatan sulfate proteoglycan II | AF038127 |
| 8.87 | CD464576 | Interleukin-8 | XM_170504 |
| 8.81 | BM735098 | Glia maturation factor, γ | NM_004877 |
| 8.75 | BM781374 | Fibulin 1 | BC022497 |
| 8.68 | CD535463 | ρ GDP-dissociation inhibitor 2 | Ξ69549 |
| 8.64 | CD465406 | KIAA0882 protein | XM_093895 |
| 8.02 | BM735336 | Unknown | AK090519 |
| 7.86 | BM734930 | Unknown | BC012423 |
| 7.81 | AY114351 | Equine granulocyte chemotactic protein 2 | AY114351 |
| 7.24 | CD536651 | Unknown | BC036098 |
| 7.2 | BI961242 | Colony-stimulating factor 3 | NM_172219 |
| 7.16 | CD466975 | Unknown | BD109582 |
| 6.93 | CD535197 | NORE1 protein | NM_031437 |
| 6.8 | CD467520 | Cyclin D2 | NM_001759 |
| 6.78 | BI961361 | KIAA0882 protein | XM_093895 |
| 6.76 | CD536618 | Cyclin D2 | NM_001759 |
| 6.6 | BI961105 | PRG1 gene | X96438 |
| 6.56 | CD469180 | FLJ00024 protein | AK024434 |
| 6.56 | BI961594 | Tumor necrosis factor, □-induced protein 6 | NM_007115 |
| 6.41 | CD468109 | □-2-microglobulin gene | □I□□□□ |
| 6.41 | CD536657 | Guanylate binding protein 1 | NM_002053 |
| 6.21 | CD469026 | Epithelial stromal interaction 1 | NM_033255 |
| 6.11 | AF503365 | Equine granulocyte colony-stimulating factor | AF503365 |
| 6.11 | BM780519 | Serine (or cysteine) proteinase inhibitor | NM_000062 |
| 6.03 | CD472099 | Junctional adhesion molecule 1 | NM_144504 |
| 5.97 | CD464893 | Immediate early response 3 | NM_003897 |
| 5.9 | BI961310 | Chemokine (C—X—C motif) ligand 5 | NM_002994 |
| 5.86 | CD464588 | B-cell CLL/lymphoma 3 | NM_005178 |
| 5.57 | CD536610 | Unknown | BC036098 |
| 5.53 | AY005808 | Equine toll-like receptor 4 | AY005808 |
| 5.49 | CD471341 | MHC class I antigen HLA-A | U03754 |
| 5.47 | CD468091 | Unknown | CD468091 |
| 5.46 | CD469607 | Hypothetical protein FLJ39885 | NM_152703 |
| 5.4 | CD528897 | N-myc (and STAT) interactor | BC001268 |
| 5.38 | BM735180 | Unknown | AX466510 |
| 5.31 | CD467650 | Unknown | CD467650 |
| 5.24 | BI960830 | Interferon regulatory factor 1 | XM_034862 |
| 5.22 | BI961018 | Immediate early response 3 | NM_052815 |
| 5.06 | CD465968 | Kinesin family member 5B | BC009353 |
| 5.04 | CD528326 | Neutrophil cytosolic factor 1 | XM_170516 |
| −5.29 | BM734828 | Dudulin 2 (FLJ10829), mRNA | NM_018234 |
| −5.38 | CD466561 | PDZ and LIM domain 2 | NM_021630 |
| −5.55 | BI961715 | Unknown | BC019236 |
| −5.64 | BM780462 | 3′-phosphoadenosine 5′-phosphosulfate | AF160509 |
| | | synthetase 2 | |
| −5.74 | CD469298 | Unknown | XM_041375 |
| −6.1 | BM735590 | Unknown | BC010959 |
| −6.32 | AJ319907 | Equine fibroblast growth factor receptor | AJ319907 |
| −6.58 | BI961854 | Heparan sulfate (glucosamine) 3-O- | NM_006041 |
| | | sulfotransferase 3B1 | |
| −6.73 | CD528599 | Transcription elongation factor A | XM_114075 |
| −7.3 | CD528582 | Ribonuclease, RNase A family, 4 | NM_002937 |
| −7.31 | BM780574 | Metallothionein 2A | BC007034 |
| −10.22 | CD466107 | Inositol 1,3,4-triphosphate 5/6 kinase | NM_014216 |
| −11.11 | BM735117 | Unknown | BC027258 |
| −12.02 | CD468788 | Unknown | CD468788 |
| −12.11 | BM780841 | E74-like factor 1 | NM_172373 |
| −18.18 | BI961458 | Smcx homolog | NM_004187 |
| −18.94 | CD535871 | Eukaryotic translation initiation factor 2, subunit | NM_001415 |
| | | 3 γ | |
| −27.75 | L42623 | MHC class I mRNA | L42623 |

*Refer to Genbank (Available at www.ncbi.nih.gov. Accessed on Jun. 15, 2003) for more information on the names and abbreviations of the provisional annotation genes.

Discussion

In the study reported here, we used a computer-based approach to create a gene expression microarray for a particular species. We then constructed and tested the performance of an equine species-specific microarray. Genetic information has been increasing dramatically since the development and use of expression microarrays; however, algorithms to examine the 3′ biased sequences have not been described to assist with generating ideal sequences for use on these arrays. Our goal was to curate all quality equine sequence data and prune the number of sequences to generate unduplicated annotated sequences for an optimized array. Our approach involved 2 major steps: identification of the 3′CDs and 3′ ESTs and subsequent annotation of the sequences. For our algorithm, the 3′ equine CDSs were identified by selecting the full and partial CDSs that had a stop codon at the 3′ end. This approach ensured that sequences selected were anchored to the 3′ end. Most would contain the 3′ untranslated region (UTR), which is more species-specific, compared with the coding region (Affymetrix. Genechip CustomExpress array design guide. Available at: http://www.affvmetrix.com/support/technical/other/custom_design_manual.pdf. Accessed Dec. 15, 2003). Because the UTR is found in many mRNA samples isolated by use of poly-dT primers, species-specific sequence heterogeneity in the UTR enhances the accuracy of species-specific arrays (Higgins M A et al., Toxicol Sci 2003; 74:470-484). Polymerase activity fades toward the 5′ end; thus, it would be possible to have a portion consisting of the UTR and none of the CDS in the processed mRNA samples. Therefore, use of the UTR sequence in probe design is an asset for improvement of microarray accuracy.

We chose to perform an algorithm analysis for the partial equine CDSs and ESTs with those in a human-mouse CDS database we created, rather than a nonredundant database available at NCBI. Our human-mouse CDS database was actually a subset of the nonredundant database and consisted of annotated human or mouse CDSs. Results of the algorithm on the basis of comparison with the annotated human or mouse CDS database would be more useful in determining the equine EST annotation and sequence orientation. Our human-mouse CDS database was much smaller than the nonredundant database, and the computing time was tremendously reduced for the algorithm.

Quite often, a single gene may be represented by several sequences, each with a unique public database accession number. The same gene may be sequenced and deposited by several laboratory groups, or the gene sequences may initially be deposited into the public database as partial CDSs, and subsequently be deposited again as complete sequences. Therefore, multiple sequences, although each with a unique accession number, will actually represent the same gene. To solve this problem, cluster programs have been designed to reduce sequence duplicates. Our cluster program models a program from the NCBI (Pontius J U et al., In: NCBI Staff, eds. The NCBI handbook. Bethesda, Md.: National Center for Biotechnology Information; 2003; 21.1-21.12). Alternatively, we could have used that NCBI cluster program, or other programs could have been incorporated into our algorithm. We chose a high filter of 95% for CDS to reduce the risk of losing fully annotated, separate, but closely related, genes (e.g., calcitonin gene related peptide I and II). We also chose a relatively high filter of 90% for ESTs to reduce the risk of duplicates and maximize the space available on the microarray to enable us to include as many genes as possible.

To maximize the number of candidate genes that could be selected for the microarray, all 3′ sequences (or close to 3′ sequences) were identified. Because transcript sequencing was performed on many cDNA libraries by use of oligo-dT primers in the first-strand cDNA synthesis (Weiss G B et al., J Biol Chem 1976; 251:3425-3431; Hagenbuchle O et al., J Biol Chem 1979; 254:7157-7162), the sequence information from these ESTs contained the polyA tail only when the sequencing process reached to the 3′ end. However, when the sequencing was initiated at the 5′ end and stopped in the middle, the obtained sequence information may not have included the polyA tail, although it may have been extremely close to the 3′ end. Therefore, ESTs characterized as no polyA may not necessarily mean that they did not contain a polyA or that the polyA was close to the 3′ end. To capture these sequences, ESTs were selected that claimed those with and without polyA to maximize the pool of candidate 3′ ESTs. The pool of sequences that did not contain the 3′ end were subsequently analyzed by use of an algorithm and compared with our human-mouse CDS database to locate the sequence position relative to the 3′ end. Any sequences within 500 bp of the 3′ end of the matched sequence were also included as a candidate for inclusion on the microarray.

The ESTs are short sequences that represent only fragments of genes or incomplete CDSs, and they may be in a sense or antisense orientation. Therefore, a major effort and emphasis was focused on how best to annotate these ESTs. In fact, we initially annotated the equine ESTs by use of an algorithm by comparison with the nonredundant database of the NCBI (data not shown). However, there were an overwhelming number of possible matches identified between the ESTs and sequences without much useful information because the matches were with chromosomal sequences, such as cDNA clones. Therefore, analysis by use of the algorithm was modified by creating our human-mouse CDS database that contained more concentrated annotated human and mouse CDSs. As a result, approximately 92% of the ESTs had matches in the algorithm analysis, and putative annotations were performed (Table 4).

This work is the first microarray accumulation of equine annotated genes and ESTs and all that are currently publicly available for horses. The equine gene expression microarray includes equine gene sequences that function in apoptosis, the cell cycle, signal transduction, and developmental biological processes (Escribano J and Coca-Prados M, Molecular Vision 2002; 8:315-332; Lo J et al. Genome Res 2003; 13:455-466). This equine array was used to evaluate the gene expression pattern of equine synoviocytes and the response to LPS, which is an established signal molecule generated by gram-negative bacteria that can be used to assess microarray function. The microarrays reported here revealed gene expression patterns typical of other custom arrays (Higgins M A et al. Toxicol Sci 2003; 74:470-484) and had excellent reproducibility of performance (r, >0.99). Very few (<4%) of the genes were expressed at such a low intensity that replicate arrays could not consistently distinguish an expressed gene from a nonexpressed gene, and all were at low to very low signal intensity (FIG. 2).

Therefore, significant discrepancies in gene expression were not identified, and high accuracy for expressed genes among replicates is anticipated with this array. Investigations that place importance on genes with low or marginal expression should perform the microarrays in triplicate or validate findings by use of methods (e.g., quantitative real-time polymerase chain reaction techniques).

The gene expression rate of approximately two thirds or greater for the microarray reported here is greater than that for human (40% to 50%) (Affymetrix. Technical documentation page. Technical note: design and performance of the GeneChip human genome U133 plus 2.0 and human genome U133A 2.0 arrays. Available at: http://www.affymetrix.com/support/technical/technotes/hgu133_p2_technote.pdf. Accessed Oct. 15, 2003) and canine (28%) (Higgins M A et al. Toxicol Sci 2003; 74:470-484) microarrays and is appropriate for sequences selected from multiple tissue libraries. These rates of expression will offer sufficient availability on the microarray for genes with no, low, or high expression. This permits evaluation for tissue-specific expression or manipulation experiments in which investigators want to optimize the detection of switched-on genes or genes that are not naturally expressed. This reveals a potential advantage of sequence-based gene selection for microarrays, compared with use of tissue-specific microarrays, in the discovery of new genes.

### Example 3

### Effect of LPS on Gene Expression as Measured on Microarray

Addition of LPS at a concentration of 100 ng/mL to synoviocyte cultures induced large-scale upregulation of many genes, most notably TNF, IL-8, prostaglandin endoperoxide synthase 2, nuclear factor kappa, interferon, and matrix metalloproteinase-1 (Table 6). Similar inflammatory genes (e.g., ILs, TNF, and cyclooxygenase-2 (a prostaglandin synthase)) reportedly increase with exposure to LPS (Hashimoto et al. Scand J Infect Dis 2003, 35:619-627; Rodgerson D H et al. Am J Vet Res 2001, 62:1957-1963). Understanding the interrelationships of these genes and unveiling the complexities and regulatory roles of these genes will require many additional studies.

Identification of a panel of 102 genes with altered expression in response to endotoxin documents the complexity of cellular signals. Up-regulation of toll-like receptor, oncogenes, IL-8, IL-1, TNF genes, interferon regulatory factor, prostaglandin endoperoxidase synthase-2, chemokine ligand, fibroblast growth factor 2, granulocyte chemotactic protein, colony stimulating factor, and similar proinflammatory molecules were anticipated. Interesting findings that will precipitate additional studies were the upregulation of chorionic gonadotropin and steroidogenic factors that may cross-communicate with stress-induced genes. Additionally, genes associated with adhesion (e.g., junctional adhesion molecule, dermatan sulfate, and heparan sulfate sulfotransferase) may be associated, assuming it happens in other equine cells, with the induction of cell adhesion classically associated with peripheral margination of WBCs in horses exposed to LPS (Palmer J L and Bertone A L, Equine Vet J 1994; 26:492-495). Analysis of our results identified a gene expression panel associated with LPS challenge exposure.

Use of the microarray to identify a subset of gene sequences highly sensitive and accurate in detecting synovial cell reaction to LPS inflammation.

For this experiment we cultured in monolayer, using techniques described elsewhere in this document, normal synovial cells from three horses. After growth to confluence the cells were exposed to 6 concentrations of LPS-Escherichia coli 055:B5 at 6 doses for 2 hours (0.01, 0.1, 1.0, 10, 100 and 1000 ng/ml). Experiments were run in triplicate. Cells were harvested at 24 hours and RNA extracted as described previously in this document for synovial cells and processed on the microarray. There were many genes that were identified to be up-regulated by the LPS across many of the doses, many that were duplicated across dosages of LPS. In final analysis, there were five genes that were up-regulated in all dosages except the lowest dose and followed a pattern that was correlated to dose; as LPS dose went up, the induction of gene expression went up. These five genes represent a very accurate signature for LPS joint inflammation at any dose and highly sensitive to detection of the gene changes. (Table 7.)

TABLE 7
Signature Genes for LPS Joint Inflammation

| GenBank | Full or | Dose LPS ng/mL | | | | | |
|---|---|---|---|---|---|---|---|
| Accession Eq. | Provisional | Fold-Change | | | | | |
| Sequence | Annotation | 0.01 | 0.1 | 1.0 | 10 | 100 | 1000 |
| CD536631 | GR02 Oncogene | — | 8 | 7 | 10 | 164 | 15 |
| AF053497 | Equine melanoma | — | 3.5 | 4.3 | 11 | 130 | 15 |
| | growth-stimulatory | | | | | | |
| | activity homolog | | | | | | |
| CD468799 | GR03 oncogene | 3 | 9.8 | 8.5 | 21 | 108 | 34 |
| BM734883 | Chemokine | — | 4.3 | 3.7 | 24 | 30 | 16 |
| | (C-C motif) ligand 7 | | | | | | |
| BL961535 | Interleukin-8 | — | 5 | 5 | 18 | 29 | 28 |

### Example 4

### Validation of the Microarray with RT-PCR

The performance of the array was also validated by comparison to quantitative real-time reverse transcription polymerase chain reaction (RT-PCR; ABI PRISM 7000™ Sequence Detection System by Applied BioSystems, Foster City, Calif.) using an equine synoviocyte LPS model of cell stimulation. Total RNA was extracted from synoviocytes using a commercially available kit (RNEasy®, QIAGEN, Inc., Valencia, Calif.) that had been stimulated with 10 ng or 1000 ng of LPS in our published manner (Gu and Bertone, AJVR: 65; 12:1664-1673, 2004). Reverse transcription of total RNA to complementary DNA (cDNA) was performed by adding random hexamers and a 10-mM deoxynucleotide triphosphate (dNTP) mix to each total RNA sample and heating to 65° C. for 5 minutes. Samples were then placed on ice and subjected to a single, brief pulse centrifugation at 4° C. A commercially available buffer (250 mM Tris-HCl, 375 mM KCl, 15 mM MgCl₂), RNase inhibitor, and 0.1M dithiothreitol (DTT) (Invitrogen Corp., Carlsbad Calif.) were added to each sample and the contents of each tube were gently mixed. Samples were incubated at room temperature for 10 minutes, then at 37° C. for 2 minutes. Moloney murine leukemia virus reverse transcriptase (Invitrogen; 200 units, diluted in 3 μl of RNase-free water) was added to each sample. Samples were mixed and incubated at 37° C. for 50 minutes. The reaction was inactivated by heating samples at 70° C. for 15 minutes. Resulting cDNA samples were frozen at −20° C.

The mRNA sequences for the genes tested (See Table 8) were amplified by the 5′-nuclease assay, using sequence specific probes labeled with the fluorescent reporter dye 6-carboxyfluorescein (FAM) on the 5′ end of the probe and the quencher dye 6-carboxytetramethylrhodamine (TAMRA) on the 3′ end of the probe to quantify accumulating accumulating PCR product in real time. Taqman® Universal PCR Master Mix, Assays-on-Demand Gene Expression Array Mix™ (containing the forward primer, reverse primer, and labeled probe for each amplicon) were added to each cDNA sample, which was diluted in RNase-free water to yield a total reaction volume of 50 μl. The thermal cycling parameters were as follows: 2 minutes at 50° C., 10 minutes at 95° C., and 40 cycles between 15 seconds at 95° C. and 1 minute at 60° C. Other techniques for the isolation and processing of RNA for RT-PCR could be used. Samples were processed and analyzed by these two gene expression techniques, the microarray and RT-PCR.

The data in Table 8 below demonstrates that the fold change in gene expression was similar quantified similarly by both RT-PCR and microarray methods.

TABLE 8

| | Synoviocytes Stimulated | | | Synoviocytes Stimulated | | |
|---|---|---|---|---|---|---|
| | 100 ng/mL LPS | | | 1000 ng/mL LPS | | |
| | TNF- | | PG | | | PG |
| | protein | | peroxide | TNF- | | peroxide |
| Genes | 6 | IL-1 | synthase | protein 6 | IL-1 | synthase |
| Microarray | 5 | 16 | 3 | 6 | 34 | 3 |
| Fold- | | | | | | |
| Change | | | | | | |
| RT-PCR | 6 | 155 | 2 | 10 | 290 | 4 |
| Fold- | | | | | | |
| Change | | | | | | |

### Example 5

### Equine-Specific Large-Scale Gene Expression Analysis of Developmental Bone Diseases

Developmental Orthopedic Disease (DOD) represents a group of bone diseases that manifest during growth and development and include articular dyschondroplasia (osteochondrosis dessicans, OCD) and cervical vertebral malformation (CVM). The underlying pathogenesis is altered endochondral ossification of mineralizing cartilage. Site-specific clinical syndromes result. Abnormalities at the articular growth front result in a dyschondroplasia called osteochondrosis dessicans (OCD) or intra-articular cartilage flaps with abnormal underlying bone. The incidence of articular osteochondrosis is increasing and the condition is present in the horse population at high levels (10-25%). OCD induces arthritis and lameness and is usually treated surgically. The hock and stifle are the most common joints affected. Abnormalities of vertebral growth result in narrowing of the cervical vertebral canal in combination with malformation of the vertebra. The result is spinal neurologic disease characterized by ataxia and weakness.

The syndrome is termed cervical vertebral stenotic myelopathy (CVM) and is treated with anti-inflammatory medication, nutritional support, and, in selected cases, surgical cervical fusion. CVM is the leading cause of noninfectious spinal cord ataxia in the horse and affects 2% of the Thoroughbred population. Both conditions are distributed internationally, in multiple breeds and usually manifest in the young growing horse. Studies supporting a genetic predisposition to both conditions, and unique biochemical and molecular features of osteochondrotic cartilage in horses, suggest that evaluation of gene expression will be a productive approach to identifying the presence and predisposition to this disease. The use of microarrays for gene expression studies and diagnostics is becoming well established. The use of a species-specific microarray is of critical importance for accurate biomarker identification and monitoring of highly specific markers. In cross-species hybridization on microarrays, even single nucleotide mismatches can alter the detectable gene expression and relative intensities resulting in erroneous conclusions. Affymetrix is a recognized manufacturer of large-scale microarray technology that is sensitive, specific, and highly repeatable.

In this Example, we describe how to quantify and bioinformaticly analyze gene expression alterations associated with two of the most common developmental orthopedic diseases in young horses, articular dyschondroplasia (osteochondrosis dessicans, or OCD) and cervical vertebral malformation (CVM). Gene expression markers were identified that uniquely identified the presence of these disease conditions (a signature). This example describes the construction of a bioinformatic tool that can predict, diagnose, and monitor therapy of these conditions. First, gene expression has been bioinformatically profiled to identify a gene expression signature for OCD and/or CVM (two forms of DOD) for use as a diagnostic tool.

To determine a gene expression profile for DOD, we collected, in 2002, data on a preeminent Kentucky thoroughbred farm (>100 foals/year) in collaboration with the farm veterinarians. Thirteen yearlings with OCD and 7 age- and sex-matched yearlings (within a month of age); and 6 weanlings with CVM, 3 weanlings with CVM affected siblings, and 4 age and sex match control weanlings were selected for the study and their medical records evaluated by a veterinarian, copied for this study and filed. All OCD horses had either stifle or hock OCD, diagnosed by radiographic lesion and the presence of affected joint effusion, that was classical for the disease. All horses with CVM had cervical spinal radiographs and a myelogram that confirmed spinal cord compression and classical malformation of the vertebrae typical of the disease. All horses with CVM had a complete neurologic examination performed previously and were neurological. Additionally all CVM horses were evaluated by a veterinary neurology specialist at the time of sample collection and showed neurological signs. All control horses had similar radiographs that were normal, had no history of joint effusions or lameness or neurologic signs and did not have any signs at the time of sample collection.

Blood was drawn by two veterinarians into three heparin tubes, placed on ice, and immediately carried to Bertone's lab for processing. Alternatively, other samples could have been obtained and similarly analyzed such as synovial fluid from the joints or cerebral spinal fluid. Blood samples from all horses yielded high quality RNA from blood (O.D. 260/280>2.0) that was frozen at −80° C. The investigators collected and copied all clinical data including radiographs, myelograms, lameness, and neurologic examinations and filed them for the study. Gene expression analysis using the equine-specific microarray, prepared as described elsewhere in this document, was performed on five DOD horses and five matched control horses.

For these studies, cells from these blood cells were isolated by centrifugation (and manual buffy coat fractionation and subsequently batch processed for ribonucleic acid (RNA) extraction, cDNA synthesis, in vitro transcription, RNA amplification and fragmentation, and RNA fluorolabeling as per the GeneChip Expression Analysis Technical Manual, Affymetrix, Inc., 2001. All equipment (Affymetrix hybridization chamber, fluidics station, and computer workstation and software) are publicly available.

For blood samples, the RNA was extracted from the white blood cells in the buffy coat by the standard method already described for synoviocytes. Blood was collected as plasma in heparin tubes to prevent clotting and consumption of cells. After centrifugation of the blood for 10 minutes (4° C.), the white buffy coat layer at the junction of plasma and packed red cells was removed carefully with a pipette and placed in RNAase free tubes and kept on ice. Buffy coat cell RNA was extracted by Trizol homogenization. Cells were suspended and homogenized/vortexed in 1 ml cold Trizol reagent for 15 seconds. 100 μL of Chloroform was added and vortex-mixed until a creamy pink color. The preparation was spun at 14,000 RPM range can be 13,000-16,000 G at 4° C. for 15 minutes. The aqueous phase (clear fluid on top) was removed in 100-μL aliquots and put in a new RNA free chilled tube (200-300 μL total). This was done carefully to not disturb the interface where DNA accumulates. 1.5-2× isopropanol was added to aqueous phase, vortex mixed and RNA precipitated at −80° C. for at least 30 minutes. After thawing to room temperature and tube inversion mixing, tubes were spun at 14,000 G at 4° C. for 30 minutes to localize the precipitated RNA at the bottom of the tube. Isopropanol was decanted and the tube towel dried for 15 minutes. The RNA pellet was redissolved in 15-25 μL of RNase-free water. The optical density concentration of RNA is measured using 2 or 4 μL of sample to 1 ml water in cuvette and reading in a spectrophotometer at 260 nm wavelength. Reading is the concentration of RNA in μg/μL.

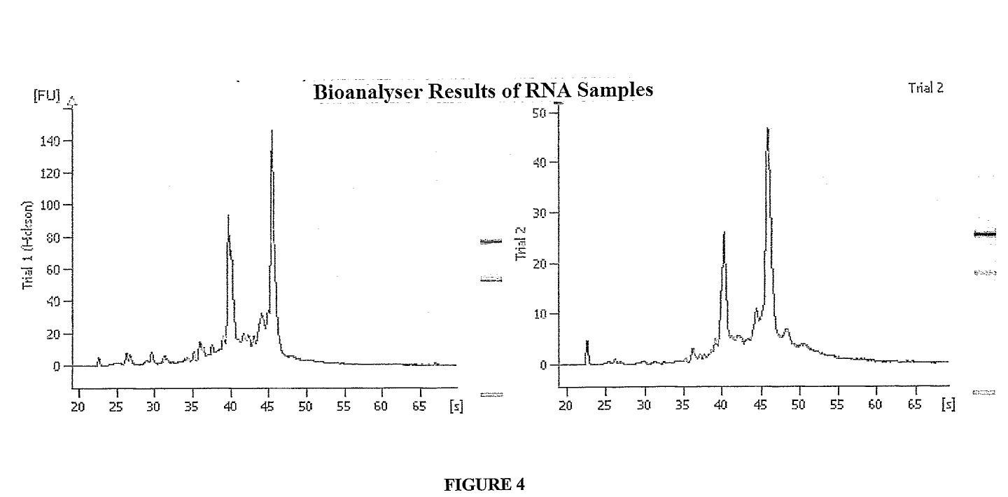

RNA was then assessed for purity by gel electrophoresis or a bioanalyzer analysis before processing for use on the microarray. It was important to have RNA of the highest integrity when using microarray to study gene expression. Even partial degradation of RNA can result in bias of quantification of different transcripts due to the variability of messenger RNA degradation. High quality RNA was also necessary for successful In Vitro Transcription (IVT) reaction during the microarray protocol to produce biotin-labeled RNA. Running total RNA in capillary electrophoresis (bioanalyzer analysis) was the most effective test for RNA quality. Capillary electrophoresis was performed using the Bioanalyzer 2100 (Agilent) and prominent 18S and 28S rRNA peaks showed high integrity of RNA (see FIG. 4). High-quality total RNA was extracted using the Trizol technique.

In some cases, the RNA was visualized for quality by electrophoresis in a 1.0% agarose gel stained with 3 μg/mL of ethidium bromide (Sigma). Gel electrophoresis was conducted at 100 volts for 30 minutes. RNA was visualized using ultraviolet transillumination (Spectroline® ultraviolet transilluminator, Spectronics Corporation, Westbury, N.Y.) in a commercially available gel documentation system (Kodak EDAS 290, Eastman Kodak Company, Rochester, N.Y.) and dedicated software (Kodak 1D Image Analysis Software, Version 3.6.0).

Labeled RNA was hybridized to equine species-specific high density DNA probes and scanned for gene expression intensity using an Affymetrix Gene Expression System and the equine custom microarray described in Example 1. Briefly, the resuspended total RNA was reverse transcribed into copy single stand DNA (cssDNA) using Superscript II reverse transcriptase (invitrogen, Inc) and T7-(dT)₂₄ primers (Affymetrix, Inc). Biotinylated copy RNA (cRNA) was formed using a Bioarray T-7 Polymerase Labelling Kit (Enzo, Inc) and then fragmented before hybridization on the GeneChip. An overnight hybridization was followed by washing and staining of the microarray with phyocerythrin. The phyocerythrin only fluoresces with cRNA that hybridized with the probe on the GeneChip. Signal intensity was then detected and measured by the microarray scanner and results were analyzed by bioinformatics software.

This equine gene expression microarray represents 3,098 equine genes that contain a bias for musculoskeletal relevance. Over 360 genes represent cell signaling functions, 322 are enzymes, 154 in protein synthesis, 375 in RNA/DNA binding including transcription factors, 193 in cell differentiation including developmental protein function, and 24 in apoptosis pathways. All known relevant genes to OCD in horses, such as PTHrP, Indian hedgehog, bone morphogenetic proteins, and receptor-activated nuclear factor kappa β ligand (RANK L) are on the array.

Bioinformatic analysis of gene intensity data by cluster analysis and comparisons among groups (OCD/CVM vs control; was performed using, initially, Affymetrix Microarray Suite Software packages, Microarray Suite (MAS) 5.0, MicroDB, and Data Mining Tool (DMT) 3.0. Probe level data was further analyzed using dChip software Li, C., and W. H. Wong. 2003. DNA-Chip Analyzer (dChip). In The analysis of gene expression data: methods and software. G. Parmigiani, E. S. Garrett, R. Irizarry, and S. L. Zeger. Springer-Verlag. Array normalization was performed using the invariant set procedure. Then, model-based expression indices (MBEI) were computed using the perfect match only model.

Genes that were significantly up- or down-regulated in DOD are listed below from the most sensitive genes to least sensitive genes to represent equine DOD. These genes, individually or in subsets of 5, 10, or 13 genes, can represent DOD to a greater or lesser accuracy and sensitivity. Due to the tight selection of control horses, these represent a direct marker of DOD.

TABLE 9

| | | | | Fold | | |
|---|---|---|---|---|---|---|
| Parametric | p-value | Norm | OCD | Change | Unique id | Gene Name |
| 1 | 9.21e−05 | 1567.2 | 542.9 | 2.887 | GBEQ1361 | Interferon -induced |
| | | | | | | protein |
| 2 | 0.0001637 | 1339 | 858.5 | 1.56 | GBEQ3012 | NFAT-activation |
| | | | | | | molecule |
| 3 | 0.0003515 | 1051.4 | 734.1 | 1.432 | GBEQ2386 | tumor differentially |
| | | | | | | expressed gene |
| 4 | 0.0008141 | 1297.1 | 847.4 | 1.531 | GBEQ3111 | |
| 5 | 0.0008758 | 1532.3 | 982 | 1.56 | GBEQ0534 | Receptor retinoic acid |
| 6 | 0.001096 | 286.9 | 169.5 | 1.693 | GBEQ3177 | |
| 7 | 0.0013346 | 532.8 | 306.5 | 1.738 | GBEQ3110 | |
| 8 | 0.0014709 | 1173.2 | 1645.1 | 0.713 | GBEQ1535 | Dendritic cell protein |
| 9 | 0.0015773 | 1345.8 | 845 | 1.593 | GBEQ0169 | Horse serpin M91161 |
| 10 | 0.0015997 | 2145.5 | 1525.9 | 1.406 | GBEQ3006 | |
| 11 | 0.0018568 | 1296.4 | 890.8 | 1.455 | GBEQ0033 | Natural resistence |
| | | | | | | macrophage associated protein |
| 12 | 0.0019245 | 256.1 | 182.8 | 1.401 | GBEQ1928 | retinoid inducible |
| | | | | | | serine carboxypeptidase |

In summary, the use of the microarray has created a method to evaluate blood of horses and identify the presence of DOD.

### Example 6

### Identification of Gene Expression Profiles for Equine Osteoarthritis

The goal of this Example was to determine a gene expression profile to identify osteoarthritis (OA), and therefore produce a gene expression signature for OA using horse samples. Osteoarthritis is one of the most significant causes of locomotor morbidity in horses and humans, with an increasing prevalence in an ageing society. To date, inflammatory and degradative pathways associated with OA have been studied in isolation. Current microarray technology permits identification and classification of cartilage molecular phenotype in large scale and can be used to unveil the complexities of the degradative pathways and discover potential intervention points for disease-curtailing therapy.

Briefly, horses were screened for OA by clinical inclusion criteria and placed into normal or OA groups. Articular cartilage of the distal metacarpal condyle was digitally photographed and harvested for mRNA analysis and histological grading. Total RNA was processed and placed on the equine gene expression microarray (Example 1). Genes with significant increases and decreases in gene expression in OA as compared to normal articular cartilage were identified as profile gene candidates. Genes were identified that changed in accordance with OA and represent an OA gene expression signature. See Tables below.

Specific Aims:

Large-scale gene expression profiling has not been applied to the study of equine osteoarthritis (OA). Although molecular pathways in OA have been studied in isolation, large scale bioinformatic analysis of gene expression has not been used to unveil the complexities of the degradative pathways. Our hypothesis is that there will be a sub-set of genes with significant up- and down-regulation in osteoarthritic cartilage as compared to disease-free cartilage. The experimental and specific aims of this Example are: 1. to grade the histological extent of cartilage degeneration in OA and matched normal equine metacarpophalangeal (MCP) joints; and 2. to identify genes with significant changes in gene expression in OA as compared to age and site matched normal cartilage.

Significance:

OA is a significant cause of morbidity in a multitude of equine sports disciplines and has been cited as the most economically important musculoskeletal disease in performance and pleasure horses (McIlwraith C W. General pathobiology of the joint and response to injury. In Joint disease in the horse (1996) Eds McIlwraith C W, Trotter G W. Pub: W.B. Saunders Company; Frisbie D D, McIlwraith C W. Evaluation of gene therapy as a treatment for equine traumatic arthritis and osteoarthritis. (2000) Clinical Orthopedics and Related Research. 379 (S); S273-S287). Treatment of OA in humans is a billion-dollar industry. OA affects more than 70% of people over 65 years of age in the United States. (American Academy of Orthopedic Surgeons, 2002; www.aaos.org) Therapeutic intervention in any species is impeded by the inability to target agents directly to the joint with the majority of treatments being directed toward reducing the pain associated with OA. The symptomatic relief afforded by protocols such as non-steroidal and steroidal therapy is often associated with undesirable side effects (McIlwraith C W. General pathobiology of the joint and response to injury. In Joint disease in the horse (1996) Eds McIlwraith C W, Trotter G W. Pub: W.B. Saunders Company; Murray R C, DeBowes R M, Gaughan E M, Zhu C F, Athanasiou K A. The effects of intra-articular methylprednisolone and exercise on the mechanical properties of articular cartilage in the horse. (1998) Osteoarthritis and Cartilage. 6; 106-114), most notably suppression of cartilage metabolism and healing.

To facilitate the development of more effective treatment regimens and selection of new therapeutic targets, it is imperative that a greater understanding of the pathophysiology of OA is obtained. Although the disease process affects the entire joint structure, including the synovial membrane, subchondral bone, ligaments and periarticular muscles, the hallmark of destruction, and the irreversible changes, occur in the articular cartilage (Malemud C J et al. (2003) Cells Tissues Organs 174: 34-48). Many of the etiological factors responsible for the initiation of disease, such as trauma and wear, is related to the breakdown of the extracellular macromolecules and release of breakdown products from articular cartilage into the synovial fluid. Cartilage macromolecules have been demonstrated to have significant immunogenic properties (Pelletier J P et al. (2001) Arthritis & Rheumatism 44: 6; 1237-1247). Furthermore, it is increasingly appreciated that chondrocytes have the capacity to produce a variety of cytokines and mediators associated with inflammation, such as prostaglandins, nitric oxide, interleukin-1β, -6 and -8, the matrix metalloproteinases and tumor necrosis factor β. Some of the extracellular matrix genes of particular interest include Types I, II, III, IX, XI, XII and XIV collagens, proteoglycans, aggrecan, decorin, biglycan, Cartilage Oligomeric Protein and Cartilage Matrix Protein, all of which are on the equine microarray (Sandell L J (2000) Clinical Orthopaedics and Related Research 379(S); S9-S16). A limited number of these genes have been studied extensively. However, methods previously available, including reverse transcriptase polymerase chain reaction (Dumond H et al. (2004) Osteoarthritis and Cartilage April:12(4); 284-295; Gelse K et al. (2003) Osteoarthritis and Cartilage February:11(2); 141-148) and in-situ hybridization (Gehrsitz A et al. (2001) Journal of Orthopaedic Research 19; 478-481), have resulted in limitations of the number of genes investigated. The simultaneous analysis of thousands of genes under identical conditions using microarray technology will provide the initial opportunity to explore the mRNA expression profile for equine OA cartilage.

Radiography and histology have historically been the standard methods of identifying the syndrome of OA in affected joints. Radiographic assessment of articular pathology, including osteophytes and enthesopathy, is an established method for the verification of osteoarthritis (Gelse K et al. (2003) Osteoarthritis and Cartilage February:11(2); 141-148). This is a relatively poor modality as sensitivity to articular degeneration is limited to detection of bony pathology, not cartilaginous change. Histological grading systems of articular cartilage are the “gold standard” for classifying OA and have been extensively used throughout human and veterinary literature to document the severity of disease in affected cartilage (Mankin H J (1971) Journal of Bone and Joint Surgery April:53(3); 523-537). We will use these established gold standards to clarify our genes of relevance to OA.

DNA microarray technology has been recently employed to identify the expression profiles in human derived chondrocytes (Aigner T. et al. (2003) Journal of Bone and Joint Surgery 85(A): 2; 117-123; Ochi K. (2003) Journal of Human Genetics 48:177-182; Aigner T. et al. (2001) Arthritis and Rheumatism 44: 12; 2777-2789), and OA affected chondrocytes (Ochi K. et al. (2003) Journal of Human Genetics 48:177-182; Aigner T. et al. (2001) Arthritis and Rheumatism 44: 12; 2777-2789). Improved understanding of the cellular events are obtained by mapping larger scale gene expression changes that take place with the natural OA condition. Expression profiling permits the classification of genes by biological function, allowing the researcher to analyze the transcriptome. Transcriptome analysis has been shown to be beneficial in human rheumatology by identifying genes with statistically significant changes of expression, thereby allowing the identification of novel proteins in the intracellular cascade typified in OA (Lequerre T. et al. (2003) Joint Bone Spine August:70(4); 248-256; Evans C H et al. (2004) Gene Therapy February; 11(4):379-89). The potential for the discovery of novel biomarkers of disease, and thus new therapeutic targets, is an attractive goal for all researchers. Simultaneous investigations involving human and equine gene expression profiles are mutually advantageous providing shared knowledge of technical tools and interpretation approaches.

Until recently it has not been possible to produce an expression profile of equine cells because a species-specific large scale microarray was not available. Our equine DNA gene expression microarray permits the quantification of the simultaneous response of 3,098 equine genes to a disease, therapy, or experimental manipulation (Gu W, Bertone A L. Curation, pruning and annotation of the public equine nucleotide database to generate an equine gene expression microarray. (2004) American Journal of Veterinary Research Manuscript In Press). This equine gene expression microarray offers an unprecedented opportunity to identify new cytokines active in the disease process, facilitating the understanding of the pathologic mechanisms of fundamental importance to the human and animal medical communities.

Increasing knowledge of the pathogenesis of OA has focused on alterations at the molecular level, leading to the advancement of intra-articular gene therapies. The emphasis has been predominantly on the transfer of genes whose products enhance synthesis of the cartilaginous matrix, or inhibit its breakdown (Evans C H (2004) Gene Therapy February; 11(4):379-89).

In the field of rheumatic diseases, cellular modification by over-expressing anabolic factors, such as insulin-like growth factor-I or transforming growth factor beta, or inhibitors of catabolic cytokines or proteolytic enzymes has been shown to protect tissues form further destruction and stimulate tissue repair (van der Pouw Kraan T C et al. (2003) Genes and Immunity 4; 187-196). Studies in rabbit models have shown indicate that the intra-articular delivery of genetically modified synoviocytes incorporating the interleukin-1 receptor antagonist gene (IL-1 RA) and interleukin-10 gene effectively targeted multiple inflammatory effectors, thereby reducing cartilage breakdown (Zhang X et al. (2004) Journal of Orthopaedic Research July; 22(4):742-50). The use of IL-1 RA gene transfer in an equine model of OA was found to result in clinical improvement and have beneficial effect on the histological appearance of articular cartilage (Frisbie D D et al. (2002) Gene Therapy January; 9(1):12-20).

The affectivity of transgenes on tissue engineering relies on adequate test systems being available. It is essential that animal models used to study gene therapy and tissue engineering respond similarly to human tissue undergoing the same disease process (van der Kraan P M et al. (2004) Biomaterials April; 25(9):1497-504). The use of animal models to be reliably representative of human OA would be supported by verifying similar alterations in gene expression. Large-scale analysis of gene expression afforded by microarray technology will provide the opportunity to validate the use of equine models for future gene therapy investigations and potentially identify novel pathways that may be susceptible to modification in the treatment of OA in both human and animal patients.

Species Relevance:

The research is purposely oriented to the investigation of equine degenerative joint disease due to its prevalence and significance in both the equine athlete and companion horse. The equine species is chosen for the study to provide data that will be most representative of the population in question, thereby maximizing validity as no assumptions are made regarding cross-species genetic sequencing or biology. The gene expression technology utilizes equipment that is species specific, dedicated to facilitate the collection of accurate profiles. The identification of novel biomarkers of OA will be relevant to paralleled research in the human and canine fields.

Experimental Design:

a. Rationale: The equine metacarpophalangeal (MCP) joint has the largest number of traumatic and degenerative lesions of all joints of the appendicular skeleton (McIlwraith C W. General pathobiology of the joint and response to injury. In Joint disease in the horse (1996) Eds McIlwraith C W, Trotter G W. Pub: W.B. Saunders Company) and was selected due to the high degree of morbidity affecting horses of all types and disciplines. The clinical signs and histologic signs of OA are classical for any species.

b. Experimental Design:

Two MCP groups were identified: Group 1: unaffected joints (n=9) consisting of normal, healthy cartilage (control) of similar age horses as OA horses and Group 2: OA affected MCP joints (n=8). Inclusion criteria for the two groups were based on parameters of joint disease validated in previously described work (Ochi K et al. (2003) Journal of Human Genetics 48: 177-182; Aigner T et al. (2001) Arthritis and Rheumatism 44: 12; 2777-2789). In summary, control cartilage was grossly normal and harvested from sound horses with normal joint palpation and radiographs. OA cartilage was grossly abnormal and harvested from lame (grade 2/5) horses with abnormal radiographs, including osteophytes and joint space irregularity. All horses underwent lameness examination, MCP joint angle and circumference measurement and radiography.

Pre-Mortem Data:

Horses underwent a routine physical examination and basic hematology (packed cell volume, total protein). Horses with parameters outside of established normal ranges were not included.

Lameness scores were based on a scale of 1-5 (American Association of Equine Practitioners grading scale) 0. Lameness not detectable. 1. Lameness intermittent, detectable after distal limb flexion at the trot. 2. Lameness consistent when trotting. 3. Lameness detectable at the walk. 4. Severe lameness at the trot and walk. 5. Non-weight bearing at the walk.

Goniometry Pain-free range of motion was measured by use of a goniometer placed on the lateral aspect of the MCP joint. The mid portion was centrally located along the MCP joint, with one arm extending along the first phalanx and the other arm extending along the third metacarpal. The joint was flexed until resistance was met, evidenced by elevation of the horses head above the initial neutral starting position.

Radiographic Examination A standard series of radiographs (5 views) in the standing horse was assessed for presence of radiographic signs of OA by determining the prevalence of osteophytes, subchondral sclerosis and joint space narrowing (van der Kraan P M et al. (2004) Biomaterials April; 25(9):1497-504).

Circumferential Measurement The circumference of each fetlock was measured using a flexible measuring tape placed around the widest aspect of the joint, with the tape passing palmar to the basal aspect of the sesamoids.

Euthanasia and Digital Photography:

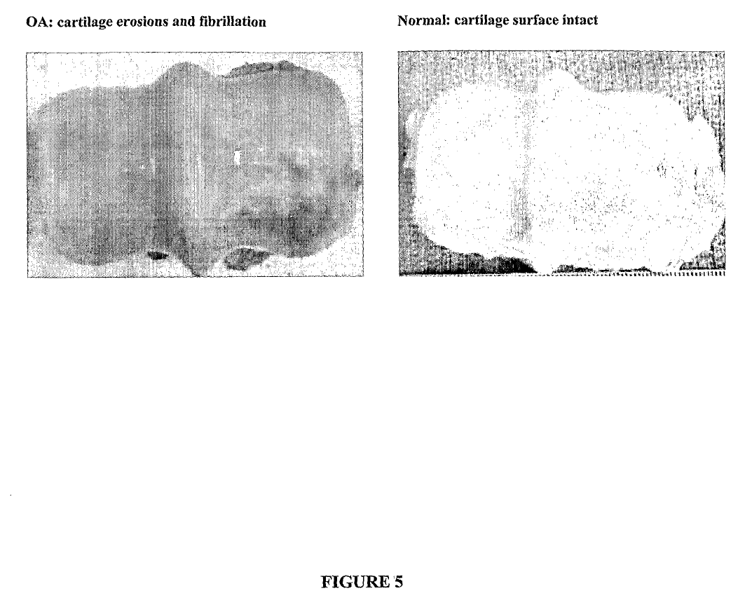

Horses were euthanized with an intravenous overdose of pentobarbital. The MCP joint was opened and the distal aspect of MC3 was assessed grossly for cartilage quality. Close high resolution digital photography with set focal distance of 150 mm documented the condition of the articular cartilage for erosions, score lines, osteophytes and surface fibrillations (Kirker-Head C A et al. (2000) American Journal of Veterinary Research 61(6):714-718). See FIG. 5, which shows exemplary articular cartilage.

Histology Cartilage biopsy specimens were harvested from representative dorsal and palmar halves and fixed immediately in neutral-buffered 10% formalin, dehydrated, and embedded in paraffin wax. Sections were cut at 6 μm, followed by HE and toluidine blue stainings as routinely described (Gelse K et al. (2003) Osteoarthritis and Cartilage February:11(2); 141-148). Slides were assessed blindly by 3 qualified individuals and allocated grades according to the descriptions below, adapted from the Mankin scoring system (Mankin H J et al. (1971) Journal of Bone and Joint Surgery April:53(3); 523-537), and mean scores documented.
a. Structure of articular cartilage through the radiate zone (0-4; 0=0% surface irregularities; 1=1-25% depth of surface irregularities; 2—26-50% depth of surface irregularities; 3—51-75% depth of surface irregularities; 4—76-100% depth of surface irregularities;
b. Cells (0—normal; 1—1-25% less cells; 2—26-50% less cells; 3—51-75% less cells; 4—76-100% less cells)

c. Matrix staining intensity (0—normal intense staining; 1—minimal reduction of staining; 2—mild reduction of staining; 3—moderate reduction of staining; 4—marked reduction of staining)

TABLE 10

| | Evaluator 1 | | | Evaluator 2 | | | | | |
|---|---|---|---|---|---|---|---|---|---|
| | | | Matrix | | | Matrix | Evaluator 3 | | |
| Sample | Structure | Hypocellularity | Stain | Structure | Hypocellularity | Stain | Structure | hypocellularity | matrix stain |
| N9LD | 0 | 0 | 0 | 0 | 0 | 0 | 0 | 0 | 0 |
| N9RD | 0 | 0 | 0 | 0 | 0 | 0 | 0 | 0 | 0 |
| N11RD | 0 | 0 | 0 | 0 | 0 | 0 | 0 | 0 | 0 |
| N11RP | 0 | 0 | 0 | 0 | 0 | 0 | 0 | 0 | 0 |
| N13LD | 0 | 0 | 0 | 0 | 0 | 0 | 0 | 0 | 0 |
| N13LP | 0 | 0 | 0 | 0 | 0 | 0 | 0 | 0 | 0 |
| N14LD | 0 | 0 | 0 | 1 | 1 | 0 | 0 | 0 | 0 |
| N14LP | 2 | 1 | 2 | 0 | 0 | 0 | 1 | 1 | 1 |
| N14RD | 0 | 0 | 0 | 0 | 0 | 0 | 0 | 0 | 0 |
| N14RP | 0 | 0 | 0 | 0 | 0 | 0 | 0 | 0 | 0 |
| N15LD | 0 | 0 | 0 | 0 | 0 | 0 | 0 | 0 | 0 |
| N15LP | 2 | 1 | 2 | 2 | 3 | 3 | 2 | 2 | 2 |
| N15RD | 0 | 0 | 0 | 0 | 0 | 0 | 0 | 0 | 0 |
| N15RP | 2 | 1 | 1 | 1 | 1 | 1 | 2 | 1 | 1 |
| N16LD | 0 | 0 | 0 | 0 | 0 | 0 | 0 | 0 | 0 |
| N16LP | 0 | 0 | 1 | 0 | 0 | 0 | 0 | 0 | 0 |
| N16RD | 0 | 0 | 0 | 0 | 0 | 0 | 0 | 1 | 0 |
| N16RP | 2 | 1 | 0 | 2 | 1 | 1 | 2 | 1 | 1 |
| OA1RP | 4 | 4 | 3 | 4 | 4 | 3 | 4 | 4 | 2 |
| OA2LP | 3 | 2 | 4 | 3 | 3 | 4 | 3 | 3 | 4 |
| OA2RD | 1 | 1 | 1 | 1 | 1 | 1 | 1 | 1 | 1 |
| OA2RP | 2 | 3 | 3 | 4 | 3 | 4 | 3 | 3 | 3 |
| OA3LD | 0 | 3 | 1 | 0 | 1 | 0 | 0 | 1 | 1 |
| OA3RD | 1 | 2 | 1 | 1 | 2 | 1 | 0 | 0 | 1 |
| OA3RP | 2 | 1 | 2 | 2 | 1 | 3 | 2 | 3 | 3 |
| OA4LD | 1 | 1 | 1 | 1 | 3 | 0 | 0 | 1 | 0 |
| OA4LP | 1 | 2 | 2 | 1 | 2 | 1 | 0 | 1 | 1 |
| OA4RD | 1 | 1 | 1 | 2 | 3 | 3 | 1 | 1 | 1 |
| OA4RP | 3 | 4 | 3 | 1 | 3 | 2 | 3 | 4 | 2 |
| OA5LP | 2 | 4 | 4 | 4 | 4 | 3 | 2 | 4 | 3 |
| OA5RD | 2 | 1 | 1 | 2 | 1 | 2 | 2 | 1 | 2 |
| OA5RP | 4 | 3 | 3 | 3 | 4 | 3 | 3 | 3 | 3 |

Code:
N = normal,
OA = osteoarthritic;
R = right,
L = left,
D = dorsal,
P = palmar,
numeral = horse number

Cartilage Harvesting For Array Analysis

The articular cartilage from distal MC3 was successfully harvested and processed completely from 6 normal and 5 OA joints. The surface was split frontally into dorsal and palmar halves and aseptically harvested using sharp curettage for snap freezing in liquid nitrogen prior to storage (−80° C.).

RNA Isolation, Amplified, Fragmentation and Labeling

Cartilage shavings were stored at −80° C. until required for RNA isolation. Cartilage was ground under liquid nitrogen using a mortar and pestle as a novel method to avoid sample thawing as has been recommended (Simmons E J et al., (1999) American Journal of Veterinary Research 60(1); 7-13). Each 1 mg of milled cartilage powder is mixed with 10 mL TRIZOL reagent (Life Technologies, Gaithersburg, Md.) and homogenized with a rotor-stator tissue homogenizer for 1 minute prior to centrifugation (Baelde H J et al. (2001) Journal of Clinical Pathology October; 54(10):778-82). The liquid phase was incubated with chloroform for phase separation. RNA was then extracted using isopropanol precipitation and one step of ethanol washing. The RNA pellet was diluted in RNase and DNase free water and amount of nucleotide calculated by measuring UV absorbance at 260/280 nm. The absorbance ratios at the different wavelengths identified if there was sufficient RNA yield or excessive sample contamination.

RNA analysis was assessed for quantity and integrity using the Agilent Bioanalyzer 2100 capillary electrophoresis unit to measure fluorescence bound to polynulceotides, ie high molecular weight RNA (OSU CCC Microarray Unit, http://www.dnaarrays.org/rna_quality.php). The degree of fluorescence provided information on DNA or salt contamination sustained during extraction, and chondrocyte apoptosis as indicated by signal intensities of 28S and 18S rRNA.

TABLE 11
Sample of RNA extraction data

| | | RNA total |
|---|---|---|
| | | yield |
| Harvest Site | A260/280 nm | μG |
| OA-01-RD | 1.956 | 13.97 |
| OA-01-RP | 1.82 | 22.46 |
| OA-02-LD | 2.05 | 18.88 |
| OA-02-LP | 2.17 | 16.04 |
| OA-02-RD | 2.24 | 22.43 |
| OA-02-RP | 1.89 | 7.93 |
| OA-03-LD | 1.81 | 8.04 |
| OA-03-LP | 2.01 | 10.70 |
| OA-03-RD | 2.23 | 8.54 |
| OA-03-RP | 2.21 | 20.42 |
| OA-04-LD | 1.98 | 18.47 |
| OA-04-LP | 2.20 | 8.02 |
| NO-09-LD | 1.88 | 10.03 |
| NO-09-LP | 2.19 | 11.24 |

Subsequent RNA preparation was as detailed in the literature (Higgins M A et al. (2003) Toxicological Sciences August; 74(2): 470-84). Total RNA was reverse transcribed into double standed cDNA using Superscript II (Invitrogen, Carlsbad, Calif.). Biotinylated cRNA is synthesized using Bioarray T-7 polymerase labeling kit (Enzo, Farmingdale, N.Y.) and fragmented prior to overnight hybridization with the equine microarray GeneChip, followed by washing and staining with Phycoerythrin. Light is emitted from the fluorescent reporter group, the bound phycoerythrin, only when it is bound to the probe. Light emitted from the perfect match oligoprobe, as compared to the single base pair mismatched oligoprobe, is detected in a scanner, which is in turn analysed by bioinformatics software (Gu W, Bertone A L. Curation, pruning and annotation of the public equine nucleotide database to generate an equine gene expression microarray. (2004) American Journal of Veterinary Research Manuscript In Press). (http://www.affymetrix.com).

Data Analysis and Results of gene expression data for OA:

Data analysis was initially performed by Affymetrix Microarray Suite Software packages (Affymetrix Custom Expression Array Design Guide. http://www.affymetrix.com), Microarray Suite (MAS) 5.0, MicroDB, and Data Mining Tool (DMT) 3.0. Probe level data was further analyzed using dChip software (Li, C., and W. H. Wong. 2003. DNA-Chip Analyzer (dChip). In The analysis of gene expression data: methods and software. G. Parmigiani, E. S. Garrett, R. Irizarry, and S. L. Zeger. Springer-Verlag). Array normalization was performed using the invariant set procedure. Then, model-based expression indices (MBEI) were computed using the perfect match only model. Probe-set level data that was called an “array outlier” by dChip was omitted and considered to be missing data in subsequent analyses. Array quality characteristics (including % array outliers and % present calls) are shown below in Table 12.

TABLE 12

| | Median | | % | % | |
|---|---|---|---|---|---|
| | Intensity | | Array | Single | GAPDH |
| Array | (unnormalized) | P call % | outlier | outlier | 3′/5′ |
| N11RD | 59 | 33.10 | 0.00 | 0.02 | 3.60 |
| N11RP | 81 | 20.40 | 0.05 | 0.19 | 5.38 |
| N13LD | 60 | 27.10 | 0.00 | 0.05 | 6.06 |
| N13LP | 57 | 28.50 | 0.00 | 0.04 | 5.29 |
| N14LD | 64 | 26.90 | 0.13 | 0.06 | 14.64 |
| N14LP | 65 | 36.60 | 0.00 | 0.02 | 6.10 |
| N14RD | 76 | 25.60 | 0.00 | 0.07 | 3.98 |
| N14RP | 69 | 25.30 | 0.05 | 0.08 | 4.91 |
| N15LD | 61 | 30.70 | 0.00 | 0.04 | 9.21 |
| N15LP | 62 | 33.60 | 0.03 | 0.07 | 3.31 |
| N15RD | 82 | 29.30 | 0.00 | 0.10 | 3.91 |
| N15RP | 62 | 26.70 | 0.00 | 0.13 | 4.33 |
| (rescan) | | | | | |
| N16LD | 96 | 28.40 | 0.00 | 0.04 | 6.07 |
| N16LP | 152 | 20.70 | 0.05 | 0.40 | 5.61 |
| N16RD | 83 | 26.30 | 0.05 | 0.02 | 3.17 |
| N16RP | 96 | 28.10 | 0.05 | 0.08 | 6.99 |
| N9LD | 109 | 21.70 | 0.05 | 0.17 | 3.24 |
| N9RD | 90 | 14.70 | 2.07 | 0.80 | 6.71 |
| OA1RP | 68 | 27.40 | 0.48 | 0.15 | 4.89 |
| OA2LP | 77 | 26.80 | 0.00 | 0.02 | 5.99 |
| OA2RDscan2 | 86 | 37.00 | 0.13 | 0.05 | 6.70 |
| OA2RP | 69 | 33.20 | 0.08 | 0.09 | 7.14 |
| OA3LD | 95 | 25.70 | 0.00 | 0.03 | 2.35 |
| OA3RD | 67 | 28.20 | 0.05 | 0.07 | 3.16 |
| OA3RP | 95 | 11.80 | 8.79 | 1.44 | 1.90 |
| OA4LD | 101 | 25.80 | 0.11 | 0.10 | 6.10 |
| OA4LP | 74 | 29.20 | 0.03 | 0.09 | 7.04 |
| OA4RD | 257 | 13.50 | 3.17 | 0.61 | 7.68 |
| OA4RP | 187 | 22.80 | 3.68 | 0.83 | |
| OA5LP | 56 | 28.30 | 0.00 | 0.04 | 5.74 |
| OA5RD | 69 | 15.30 | 3.58 | 1.12 | 4.16 |
| OA5RP | 73 | 14.40 | 4.73 | 1.14 | 3.71 |

After MBEI computation and log-transformation of the values, data were imported into BRB ArrayTools for statistical comparisons. Only probe sets that displayed a significant amount of variation in expression among specimens were considered for further analysis. Furthermore, probe sets receiving an “Absent” call for more than 75% of the specimens were omitted. These filtering criteria resulted in a set of 521 probe sets for inclusion in the statistical comparisons.

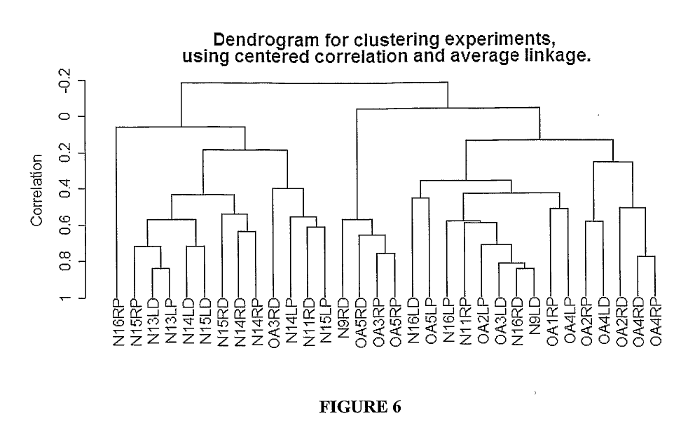

Specimens were clustered using hierarchical clustering with average linkage and one minus Pearson correlation as the distance measurement (see FIG. 6). One of the two main clusters consists almost entirely of normal specimens.

As demonstrated the samples identified as normal based on clinical and gross examination correlated and clustered in gene expression patterns. Samples classified at OA significantly clustered with OA gene expression patterns. Not all cartilage initially classified as clinically and grossly normal was completely normal on histology or as identified above in gene expression pattern. This demonstrated a continuum of cartilage expression change and that age matched controls are critical to pick up differences that are due to actual OA disease and not just aging of joints.

Statistical tests were performed at a nominal 0.002 significance level. Examining 521 probe sets at this significance level results in roughly one expected false positive claim (i.e., a probe set determined to be differentially expressed that in truth is not) under the null hypothesis of no differential expression of probe sets. Tighter control of multiple comparisons using permutation methods was also performed (Korn E L, Troendle J F, McShane L M and Simon R. Journal of Statistical Planning and Inference. 2003. 124:379-398). Permutation methods allow confidence statements to be made about the actual (as opposed to expected) number of false positive claims.

First, an interaction between aspect of joint (palmar and dorsal) and disease status (normal and OA) was tested for. For each horse's joint that included both a palmar and dorsal specimen (8 normal joints and 5 osteoarthritic joints), the difference in gene expression between the palmar and dorsal aspects was computed. A univariate t-test on each probe set was then performed, comparing normal to osteoarthritic. Many differentially expressed probe sets in this comparison would be evidence of an aspect-disease interaction. However, no differentially expressed genes resulted from this comparison.

Since there was no evidence of an aspect-disease interaction, the dorsal and palmar gene expression profiles were averaged within a particular joint and tested for differences in expression between normal and osteoarthritic joints (10 normal joints and 9 osteoarthritic joints were included in this comparison). Three probe sets were significantly differentially expressed (Table 13 below). Based on the permutation analysis, we were 90% confident that these three probe sets contained at most one false positive. Annotation of these genes, using the methodology described earlier in this document, reveals that BGEQ 0070 is Type II collagen, and GBCA0190 is Type IIA procollagen.

TABLE 13
Gene Expression Signature for OA, Regardless of Severity

| | | signal | | |
|---|---|---|---|---|
| | Parametric | intensity | signal intensity | Fold |
| Unique id | p-value | normal | OA | difference |
| GBEQ0070_s_at | 0.0002271 | 91.4 | 203.9 | 0.448 |
| GBEQ3104_at | 0.0003064 | 40.6 | 26.3 | 1.544 |
| GBCA0190_at | 0.00184 | 36.1 | 79 | 0.457 |

More appropriately, since there were no aspect/disease interactions, analyses were performed for the normal vs. osteoarthritic comparisons within palmar and dorsal aspects. These results disregarded the lack of a detectable aspect-disease interaction and results included the genes identified above. For the comparison involving palmar aspects (severe OA), eight normal and eight osteoarthritic specimens were considered. Five probe sets were significantly differentially expressed, and we were 90% confident that these five probe sets contain at most one false positive. (Table 14 below) Annotation of these genes in similar fashion to above reveal these genes represent NSF1-BP, eukaryotic translation initiation factor, beta cell CLL/lymphoma 2 gene, and heparan sulfate (glucosamine) 3-0 sulfatransferase. These genes are important in cell division and aggrecan matrix production of chondrocytes and cartilage, respectively. Down-regulation of these genes that we detected in more severe OA are signals of cell arrest in growth and matrix production.

TABLE 14

| | Parametric | signal intensity | signal intensity | Fold |
|---|---|---|---|---|
| Probe set | p-value | normal | OA | difference |
| GBEQ3104_at | 0.0003445 | 44.6 | 27.5 | 1.622 |
| GBEQ1029_at | 0.0003829 | 74.1 | 43.3 | 1.711 |
| GBEQ1212_at | 0.0005054 | 123.9 | 54.2 | 2.286 |
| GBEQ1854_at | 0.0010417 | 64.2 | 33.2 | 1.934 |
| GBEQ2019_at | 0.0013562 | 27.1 | 13.1 | 2.069 |

For the comparison involving dorsal aspects (mild OA), ten normal and six osteoarthritic specimens were considered. Four probe sets were significantly differentially expressed, and we are 90% confident that these 4 probe sets contain at most 1 false positive (Table below).

TABLE 15

| | | signal | | |
|---|---|---|---|---|
| | Parametric | intensity | signal intensity | Fold |
| Probe set | p-value | normal | OA | difference |
| GBCA0190_at | 0.0001425 | 34.6 | 89.8 | 0.385 |
| GBEQ0070_s_at | 0.0004725 | 92.7 | 261.8 | 0.354 |
| GBEQ0255_at | 0.0010854 | 9 | 22.7 | 0.396 |
| GBEQ0255_x_at | 0.0017371 | 39 | 113.2 | 0.345 |

Annotation of these genes by methodology described in this document revealed that GBCA0190 is Type IIA procollagen, GBEQ0070 is Type II collagen and GBEQ0255 is Type 1A2 collagen.

Histology scores were examined for an association with gene expression. The structure, hypocellularity, and matrix stain scores were summed for each scorer to obtain an overall histology index for each specimen for each scorer, then the median overall index was computed for each specimen. Three groupings of overall scores were apparent: a group consisting solely of normal specimens that had median overall scores of 0 (termed “low”), a group consisting of 8 osteoarthritic specimens and 4 normal specimens that ranged in score from 2 to 6 (termed “medium”) and a group consisting solely of osteoarthritic specimens that ranged in score from 9 to 11 (termed “high”). Differences in intensity of expression between the osteoarthritic joints with medium histology and high histology indices were not identified.

The annotation of differentially expressed genes was performed as described above, briefly by Blast analyses against combined human and mouse databases. (See Table 33.)

As seen in the rigorous and stringent statistical analyses performed above, several genes are up-regulated and statistically represent earlier OA (dorsal OA; less severe lesions). The upregulation of GBCA0190, GBEQ0070, and GBEQ0255 represent a signature for early OA at a statistical significance of P<0.001. Additional genes listed below were also highly associated with OA and represent a profile of less severe (dorsal) OA with less accuracy. If these genes were present in addition to the genes in table 15, this would add power to the accuracy of the gene signature.

TABLE 16

| | | signal | signal | |
|---|---|---|---|---|
| | Parametric | intensity | intensity | Fold |
| Probe set | p-value | normal | OA | difference |
| GBCA0190_at | 0.0001425 | 34.6 | 89.8 | 0.385 |
| GBEQ0070_s_at | 0.0004725 | 92.7 | 261.8 | 0.354 |
| GBEQ0255_at | 0.0010854 | 9 | 22.7 | 0.396 |
| GBEQ0255_x_at | 0.0017371 | 39 | 113.2 | 0.345 |
| GBEQ0255_s_at | 0.0024778 | 10.7 | 19.6 | 0.546 |
| GBEQ3035_at | 0.0036182 | 32 | 64.1 | 0.499 |
| GBEQ3104_at | 0.0039462 | 39.9 | 25.1 | 1.59 |
| GBEQ1633_at | 0.0060233 | 26.4 | 18.5 | 1.427 |
| GBCA0189_s_at | 0.0069451 | 22.5 | 37 | 0.608 |
| GBEQ0916_at | 0.0103856 | 81.8 | 38.3 | 2.136 |
| GBEQ1009_at | 0.0108189 | 57 | 93.9 | 0.607 |
| GBEQ1928_at | 0.0115205 | 65 | 93.3 | 0.697 |
| GBEQ0069_at | 0.012457 | 17.6 | 28 | 0.629 |
| GBEQ2816_at | 0.0126772 | 66.9 | 133.2 | 0.502 |
| GBEQ1692_s_at | 0.0138907 | 33.3 | 56.5 | 0.589 |
| GBEQ1779_s_at | 0.0179735 | 95 | 72.5 | 1.31 |

Several genes are up-regulated in early (less severe) OA 2-fold or greater which is considered significant in biologic systems, by convention. It is important to distinguish statistical and biological significance. If the genes showing biologic significance and the genes showing statistical significance are combined in smaller subsets, a greater association with OA is predicted. Evaluation of fold changes produced additional genes that represent OA in the Table below. If these genes are present in addition to the genes listed in Table 15, it may enhance the accuracy of the call of the presence of OA.

TABLE 17

| | | signal | signal | |
|---|---|---|---|---|
| | Parametric p- | intensity | intensity | fold |
| Probe set | value | normal | OA | difference |
| GBEQ0255_x_at | 0.0017371 | 39 | 113.2 | 0.345 |
| GBEQ0070_s_at | 0.0004725 | 92.7 | 261.8 | 0.354 |
| GBCA0190_at | 0.0001425 | 34.6 | 89.8 | 0.385 |
| GBEQ0255_at | 0.0010854 | 9 | 22.7 | 0.396 |
| GBEQ0052_at | 0.0530537 | 33.7 | 71.1 | 0.474 |
| GBEQ3035_at | 0.0036182 | 32 | 64.1 | 0.499 |

Only two genes were down regulated in dorsal (less severe) OA 2-fold or greater (GBEQ0776 and GBEQ0916) which is considered biologically significant. GBEQ0916 is an anti-death gene and if down regulated would result in cell death as occurs insidiously in OA. If these gene changes were present along with genes from tables 15 and 17, these might add accuracy to the call of early OA.

Additionally, several genes were up-regulated >2-fold in later more severe (palmar) OA and if these gene expression changes were present in addition to the 5 genes down-regulated that represent the signature for severe OA, Table 14, they would add power to the call of late stage OA.

TABLE 18

| | | signal | signal | |
|---|---|---|---|---|
| | Parametric p- | intensity | intensity | Fold |
| Probe set | value | normal | OA | difference |
| GBEQ0070_s_at | 0.0032033 | 86.5 | 199.5 | 0.434 |
| GBCA0155_at | 0.1416793 | 16.7 | 36.8 | 0.454 |
| GBCA0190_at | 0.0261547 | 40.5 | 83.3 | 0.486 |
| GBEQ0092_at | 0.1570649 | 14.4 | 29 | 0.497 |
| GBEQ0255_s_at | 0.0247576 | 9 | 18 | 0.5 |

Several genes were down regulated >2-fold in more severe (palmar) OA and if these gene expression changes are present in addition to the 5 genes down-regulated that represent the signature, they may add power to the call of late stage, severe OA.

TABLE 19

| | | signal | signal | |
|---|---|---|---|---|
| | Parametric p- | intensity | intensity | Fold |
| Probe set | value | normal | OA | difference |
| GBEQ2135_at | 0.1354844 | 611.8 | 250 | 2.447 |
| GBEQ1151_at | 0.0427891 | 117.2 | 48.2 | 2.432 |
| GBEQ1240_at | 0.0031178 | 129.9 | 54.5 | 2.383 |
| GBEQ0140_at | 0.0790122 | 147.6 | 62 | 2.381 |
| GBEQ0918_at | 0.0334057 | 55.8 | 23.8 | 2.345 |
| GBEQ2493_at | 0.0089043 | 132.3 | 56.5 | 2.342 |
| GBEQ2499_at | 0.1190244 | 562 | 241.6 | 2.326 |
| GBEQ1212_at | 0.0005054 | 123.9 | 54.2 | 2.286 |
| GBEQ2623_at | 0.0134986 | 323.9 | 141.9 | 2.283 |
| GBEQ2008_at | 0.0097757 | 190.9 | 85.6 | 2.23 |
| GBEQ1622_at | 0.0698789 | 371.2 | 168.4 | 2.204 |
| GBEQ1883_at | 0.0093816 | 103 | 47.4 | 2.173 |
| GBEQ2698_at | 0.038729 | 168.3 | 78.8 | 2.136 |
| GBEQ0574_s_at | 0.002315 | 107.5 | 51.7 | 2.079 |
| GBEQ2019_at | 0.0013562 | 27.1 | 13.1 | 2.069 |
| GBEQ0916_at | 0.0071843 | 95.8 | 47 | 2.038 |
| GBEQ2697_s_at | 0.0589158 | 159 | 78.2 | 2.033 |
| GBEQ1330_at | 0.0052024 | 56.2 | 27.9 | 2.014 |

In Summary, our methodology has been applied to a disease condition of osteoarthritis and identified gene signatures that represent this disease state.

### Example 7

### Selection of Viral and Protozoal Sequences for Inclusion on Microarray

Equine viral and protozoal diseases were identified for use in a diagnostic microarray. The selected organisms included equine herpesvirus 1, equine herpesvirus 2, equine herpesvirus 3, equine herpesvirus 4, equine herpesvirus 5, equine morbillivirus, Neospora hughesi, Sarcocystis neurona, and West Nile virus. Nucleic acid sequences were selected based on the following procedure.

Briefly, the herpesviruses 1, 2, and 4, and West Nile had complete genome data available in the public database. Therefore, for these, the sequences encoding capsid, membrane, envelope, or virus package proteins were specifically selected. Other viruses did not have complete genome data, so all of the available sequences were selected for those species. Table 37 lists an annotation of equine viral and protozoal sequences identified in accordance with the invention; Table 38 shows the actual sequences (SEQ ID NOS 3798-3859).

The sequences can be used as is, as the basis for a microarray, or can be separated based on pathogen and then used for generation of a microarray.

### Example 8

### Equine-Specific Large-Scale Gene Expression Analysis of Equine Protozoal Myelitis

Equine protozoal myelitis represents an infectious disease with protozoan organisms, sarcocystis neurona, canis neospora, and maybe others, that encyst in neuronal cell bodies in the central nervous system resulting in neurologic disorders in horses. The horse is a dead-end host and not a host in the primary life cycle of the organisms. Well-described clinical signs include spinal ataxia and weakness as well as muscle atrophy, peripheral nerve dysfunction, and possibly any other lower motor neuron dysfunction. Diagnosis is usually inconclusive and limited because organisms are hard to find on histology due to lesion rarity in the CNS and obviously requires death of the animal to retrieve the brain and spinal cord. Blood and cerebral spinal fluid assays to date are inconclusive because they have depended on antibody titers or staining that does not effectively distinguish exposure to organisms and pathologic invasion by the organism. Other diagnostic approaches to identify organisms have been limited by oversensitivity (high false positives) and failure to assess the biologic response to the organism as part of the cause of the development and severity of the disease.

The use of a species-specific large-scale gene expression microarray permits the simultaneous measurement of the biologic response to the organisms, which may include increased inflammatory and immunologic responses. Cells from spinal cord fluid or blood could be processed for use on the array to identify these changes and monitor response to treatment. RNA placed on the microarray provides a signature gene expression typical of the disease as compared to other neurologic diseases such as CVM previously described.

### Example 9

### Protozoan-Specific Gene Expression Microarray Analysis for Equine Protozoal Myelitis

Sequences have been placed on the array, which are genes expressed by Sarcocystis neurona and Canis neospora, similarly obtained from the public database as the sequences in Example 4 above. These S. neurona and C. neospora RNA sequences were selected to identify as high sensitivity as can be obtained on a microarray the presence of the organism and its infection in cells of the horse or other species for that matter. Since these sequences were generated from the organisms, the species from which infected tissue was obtained would not be required to be only horse.

The equine species has a significant prevalence of this disease and therefore would be a logical animal to inspect tissues. The sequences on the microarray are specific to these organisms and these organisms must have infected cells to make this RNA that would be detected on the array.

Other diagnostic tests for the presence of these organisms have attempted to detect DNA from the organisms, by PCR or other techniques. DNA is highly stable and can represent dead or silently encysted organisms. DNA-based techniques are also known for a high false positive rate due their extreme sensitivity and ease of laboratory or processing contamination. RNA, on the other hand, is labile and to be present, must be from active organisms. It does not contaminate laboratories as it is readily degraded at room temperatures.

For this study, eight adult healthy horses were used. Six horses were dosed orally with Sarcocystic neurona organisms to induce equine protozoal myelitis disease and two horses were undosed and served as controls. Horses were subsequently euthanized when clinical signs developed or at the same time period (controls). Tissues were harvested from the spinal cord, snap frozen in liquid nitrogen, stored at −80° C. and transferred to Dr. Bertone's laboratory for RNA extraction and microarray processing. RNA extraction and processing was performed precisely as outlined in the Example of stress (Example 11, below) and microarrays scanned at the Cancer Microarray Core facilities The Ohio State University.

RESULTS: Adequate quantity and quality of RNA was obtained in these samples. Statistical analysis performed by Dr. Alan Bakaletz in a similar manner as outlined in Example 11, below. Twenty-three genes had significant up- or down-regulation in the experimental horses as compared to the control horses. (Table 20.) The greatest fold change (13.4) was in gene GBEQ0486, Major histocompatibility class II. GBEQ2412 and GBEQ0393 also repesent the upregulation of the important immunomodulatory genes, integrin alpha L and leukocyte immunoglobulin-like receptor 3, respectively. We postulate that the disease is actually caused by an immune reaction to the Sarcocystis organism rather than direct destruction by the organism. This is the first documentation of this and represents a signature for the disease in horses absolutely known to have the disease.

TABLE 20
Mean Signal Intensities of Genes That Were Significantly Different
(P < 0.01) Between Control and Experimental Horses

| ProbeSet | Ctl Mean | Exp Mean | Diff. | p-Value |
|---|---|---|---|---|
| GBEQ0445_x_at | 224 | 75 | −148 | 0.00045390 |
| GBEQ0322_at | 241 | 100 | −141 | 0.00047655 |
| GBEQ2055_at | 82 | 267 | 184 | 0.00249491 |
| GBEQ0528_at | 1,318 | 925 | −393 | 0.00337740 |
| GBEQ0469_at | 452 | 228 | −223 | 0.00344998 |
| GBEQ2731_at | 142 | 727 | 584 | 0.00518644 |
| GBEQ0803_at | 1,937 | 3,393 | 1,455 | 0.00554004 |
| GBEQ2977_at | 76 | 228 | 152 | 0.00605627 |
| GBEQ0368_at | 2,297 | 3,852 | 1,555 | 0.00685735 |
| GBEQ0551_at | 58 | 364 | 307 | 0.00719710 |
| GBCA0196_at | 57 | 520 | 463 | 0.00726521 |
| GBEQ0683_at | 45 | 539 | 495 | 0.00807589 |
| GBEQ1852_at | 344 | 1,168 | 824 | 0.00840475 |
| GBEQ0996_at | 229 | 132 | −98 | 0.00840518 |
| GBCA0317_at | 40 | 167 | 127 | 0.00873921 |
| GBEQ0486_s_at | 405 | 5,429 | 5,025 | 0.00880188 |
| GBEQ1295_at | 5,395 | 3,966 | −1,428 | 0.00895785 |
| GBEQ0941_at | 887 | 1,913 | 1,026 | 0.00902847 |
| GBEQ2412_at | 324 | 1,041 | 717 | 0.00906464 |
| GBEQ2860_at | 21 | 123 | 102 | 0.00915093 |
| GBCA0393_at | 14 | 211 | 197 | 0.00946307 |
| GBEQ3111_at | 50 | 136 | 86 | 0.00975737 |
| GBCA0466_at | 181 | 831 | 650 | 0.00993219 |

The presence of the sarcocystis organism was detected by the microarray in the experimental horses. (Table 21.) Most experimental horses had increased sarcocystic RNA detection on the microarray over background in the control horse, with five of ten genes showing a 2-fold positive change ranging from 2.2 to 22.2. These data confirmed the ability of the method and the microarray to detect the presence of sarcocystis organism. Importantly, our selection of RNA confirms active infection of organism and is a unique feature of this method. We also have used a unique model that defines the presence of organism in the animal with certainty.

TABLE 21
Signal Intensity (Raw, Mean, and Fold-Change) for Control and
Experimental (Equine Protozoal Myelitis) Horses for the Ten Genes that Identify
the Equine Protozoal Organism

| | Ctrl | Ctrl | Exp | Exp | Exp | Exp | Exp | Exp | Ctrl | Exp | |
|---|---|---|---|---|---|---|---|---|---|---|---|
| Genes | 6617 | 742 | 744 | 6451 | 6453 | 6459 | 6460 | 6570 | Mean | Mean | FoldChn |
| GBEV0042 | 77.9 | 161.3 | 552.5 | 128 | 16.1 | 400 | 406 | 105 | 119.6 | 267.9 | 2.2 |
| GBEV0043 | 7.7 | 53.8 | 73.6 | 39.9 | 26.4 | 124 | 68.5 | 9.6 | 30.8 | 57.0 | 1.9 |
| GBEV0044 | 130.9 | 160.5 | 1531.8 | 315 | 537 | 1057 | 709 | 128 | 145.7 | 712.96 | 4.9 |
| GBEV0045 | 8.8 | 35.4 | 105.6 | 13.9 | 49.3 | 53 | 80.6 | 7.2 | 22.1 | 51.6 | 2.3 |
| GBEV0046 | 42.3 | 162.8 | 210.5 | 82.6 | 168 | 317 | 298 | 40 | 102.6 | 186.0 | 1.8 |
| GBEV0047 | 141.4 | 231.9 | 1254.9 | 444 | 371 | 494 | 875 | 154 | 186.7 | 598.8 | 3.2 |
| GBEV0048 | 1.2 | 2.6 | 9.1 | 17.4 | 12.5 | 157 | 25.7 | 29 | 1.9 | 41.8 | 22.0 |
| GBEV0049 | 57.5 | 574.2 | 1502.1 | 300 | 572 | 844 | 547 | 69.3 | 315.9 | 639.1 | 2.0 |
| GBEV0050 | 5.2 | 360.6 | 638.1 | 71.6 | 24.6 | 104 | 430 | 11.7 | 182.9 | 213.3 | 1.2 |
| GBEV0051 | 23.6 | 242.6 | 252.4 | 73.8 | 121 | 143 | 159 | 30 | 133.1 | 129.9 | 1.0 |

### Example 10

### Equine Viral-specific Gene Expression Analysis of Herpes Virus-1 Infection in Horses

Equine Herpes Infection is classically characterized by fever, nasal discharge (i.e., an upper respiratory tract infection) and malaise. This disease, however, can be particularly virulent with some strains, such as occurred in 2003 at Finley College Equestrian Program herd in Central Ohio. The Ohio State

University was integrally involved in containment of this outbreak and in the diagnostics.

Of 132 horses, the majority developed clinical signs >75%, and this is an exceptionally high virulence rate. Typically, most exposed horses will not develop clinical signs, but fight off the invading organism before clinical signs occur. Of these, a high percent (>10%) developed the complicating neurologic disease that is associated with this virus, documenting it as a neurotrophic strain. Diagnosis is currently dependent on serum antibody titer and viral culture from nasal swabs. The former is limited by representing past exposure only, not current disease. Therefore, serial titers are necessary to demonstrate expected increases in titers.

In all regards, these results can be influenced by previous vaccination status as most horses are vaccinated for equine herpes-1. The viral culture requires a minimum of 2 weeks and typically longerto complete. It is fraught with false positives from organisms harboring in the laboratory and contaminating long-standing culture plates. This was a problem in the diagnostic testing of this outbreak. Use of Herpes virus-1 RNA sequences on a microarray for testing offers increased sensitivity, bulk analysis, and rapid turnaround.

RNA from cells from or any other tissue suspected of containing organisms, such as spinal cord, cerebral spinal fluid cells, blood, discharges, etc. is isolated and placed on the microarray of Example 4, with appropriate control samples. The presence of herpes virus-1 RNA means that the organism is not only present but has infected cells, inserted its DNA into the cell nucleus and is using the cell machinery to make the virus's own RNA to make the virus's own proteins necessary for it to invade and replicate. In other words, the virus has infected the host, and is not just present. It currently takes three days to complete the processing for this microarray and obtain results, a substantial savings in time as compared to several weeks. The same tests can be run on equine morbillivirus, Neospora hughesi, Sarcocystis neurona, and West Nile virus.

This microarray diagnostic test also can detect infection before clinical signs even become apparent and/or carriers of the virus that are not yet clinical. Using our invention, we have demonstrated the ability of microarray to detect activation of latent herpes virus infection in horse cells. This is an example of a powerful diagnostic application for herpes infection, latent or subclinical. To demonstrate this, a normal horse, normal on physical examination without signs of Herpes virus infection, had cells submitted for culture. The RNA was extracted and put on the array. There was no expression of any of the Herpes virus genes in the initial cell cultures. However, the importance of early diagnosis includes rapid isolation of infected animals, release of uninfected animals from expensive quarantine, identification of outbreaks, and moving animals at high risk for the complications like neurologic disease and abortion.

In the case of these cells in culture from an asymptomatic horse, challenge of the cells in culture with a nonreplicating, inactivated E-1 defective human Adenovirus-5 (Bertone et al. J Orthop Res 2004; 22:1261-1270) incorporated the adenovirus DNA and transgenes into the horses cells (bone marrow derived mesenchymal stem cells) and was confirmed by ELISA measurement of gene product carried by the adenovirus. (Zachos and Bertone, Trans Orthop Res Soc Abstract No. 398; 2005.) Significant up-regulation of many genes occurred by day 2 in these cells associated with the adenoviral infection (including the transgenes carried by the virus and subsequent signaling genes, but not Herpes virus. These data confirm that initially these cells were not expressing Herpes-2 genes (Table 22 below). Data in the Table is shown as the Herpesvirus gene expression in three different adenovirus construct treated cells (Ad-BMP2; AdBMP6 and AdLacZ) expressed as a ratio to the same cells at the same day of culture without adenovirus infection.

TABLE 22
Sequences with three-fold or Greater upregulation of gene
expression in equine mesenchymal stem cells cultured for 2 days and
associated with Adenoviral transduction as compared to the same
cells without adenoviral transduction

| | | d2 | d2 | d2 |
|---|---|---|---|---|
| | | AdBMP2 | AdBMP6 | AdLuc |
| | | vs. d2 | vs. d2 | vs. d2 |
| Gene | Biological Process | NoAd | NoAd | No Ad |
| Smad6 | Regulation of transcription | 381.14 | 4.59 | — |
| | of bone morphogenetic | | | |
| | proteins | | | |
| Bone morphogenetic | Embryonic development | — | 362.04 | — |
| protein (BMP6) precursor | | | | |
| ALK5 for TGF beta | Embryonic development; | — | — | 18.38 |
| receptor type I | signal transduction | | | |
| Exostoses (multiple) 1 | Cell growth/maintenance; | — | 3.03 | — |
| (EXT1)* | glycosaminoglycan | | | |
| | biosynthesis; skeletal | | | |
| | development | | | |
| Inhibin beta A subunit | Cell growth/maintenance; | 3.48 | — | — |
| | signal transduction; skeletal | | | |
| | development; apoptosis | | | |
| Tumor necrosis factor- | Regulation of transcription; | 3.48 | — | — |
| alpha | signal transduction; anti- | | | |
| | apoptosis; apoptosis; | | | |
| | necrosis | | | |
| p53-responsive gene 1 | Anti-apoptosis; apoptosis; | 6.96 | 12.13 | — |
| (PRG1)* | cell growth/maintenance | | | |
| NFKBIA (nuclear factor of | Apoptosis | 3.48 | 5.66 | — |
| kappa light polypeptide | | | | |
| gene enhancer in B-cells | | | | |
| inhibitor, alpha)* | | | | |
| Interleukin 8 (IL8)* | Inflammation; signal | — | 3.25 | — |
| | transduction | | | |
| CXCL2 (Alias: GRO2)* | Signal transduction; | — | 3.25 | 3.73 |
| | inflammation | | | |
| Matrix metalloproteinase 3 | Collagen catabolism | 3.24 | 3.48 | — |

*Denotes annotated expressed sequence tag (EST)
TGF = transforming growth factor

Within 12 days of culture, massive upregulation of Herpesvirus 2 gene expression indicated active infection and was detected by our microarray, in some cases with several hundred fold increases in Herpes gene expression from these horse cells. Control cells from the same horse and original mesenchymal stem cells were cultured simultaneously for the same duration in directly adjacent wells without adenovirus infection to serve as tight controls and eliminate Herpesvirus contamination concerns. None of the control wells showed this increase in Herpesvirus gene expression.

TABLE 23
Sequences with 3-fold or greater upregulation of gene expression in
equine mesenchymal stem cells cultured for 12 days

| | Fold Change | | | |
|---|---|---|---|---|
| | d12 | d12 | d12 | d12 |
| | AdBMP2 | AdBMP2 | AdBMP6 | AdBMP6 |
| | vs. d0 | vs. d2 | vs. d0 | vs. d2 |
| Sequence | NoAd | NoAd | NoAd | NoAd |
| Equine herpesvirus 2 | 315.2 | 11.3 | 18.4 | 16 |
| Cartilage oligomeric | 55.7 | 14.9 | 55.7 | 12.1 |
| matrix protein (COMP)^{∞} | | | | |
| Gelsolin^{∞} | 4.9 | — | 3.5 | — |
| Angiomodulin (AGM)^{∞} | 3.7 | — | 4.3 | 3.2 |
| Plasminogen activator | — | 5.3 | 3.0 | 8.6 |
| inhibitor-1 (PAI-1)^{∞} | | | | |
| Procollagen alpha 1 (I) | — | 4.6 | 5.3 | 8.6 |
| (COL1A1)^{∞} | | | | |
| Inhibin, beta A subunit^{∞} | — | 3.0 | — | 4.3 |
| Bone morphogenetic | — | — | 59.7 | 78.8 |
| protein 6 precursor | | | | |
| (BMP6)^{∞} | | | | |
| Smad6^{∞} | — | — | 19.7 | 5.6 |
| Golgi apparatus protein^{∞} | — | — | — | 18.4 |
| Procollagen alpha-1 type | — | — | — | 4.9 |
| III precursor (COL3A1)^{∞} | | | | |
| Parathyroid hormone- | — | — | — | 4.3 |
| related peptide^{∞} | | | | |
| Keratinocyte growth | — | — | — | 3.2 |
| factor (fgf-7)^{∞} | | | | |
| Tissue inhibitor of | — | — | — | 3.0 |
| metalloproteinase-1^{∞} | | | | |

These data confirm that our microarray can detect Herpesvirus 2 infection, changes in Herpesvirus 2 infection and serve as a diagnostic indicator for Herpesvirus 2 infection. Additionally our data demonstrate that culture of cells latently infected with Herpesvirus can activate the infection and, furthermore, challenge of cells with inactivated Adenovirus, may serve as a method to rapidly diagnose carriers of Herpes infections. The challenge with Adenovirus accelerated and amplified the expression of Herpesvirus-2 in these cells.

### Example 11

### Gene Expression Patterns to Detect Potentially Compromising Stress in Horses

The present invention can be used to detect conditions in horses, not simply diseases in horses, such as the condition of stress, which is known to make animals and humans predisposed to disease. Using a model of stress in horses (Sofaly C. J. Parasitol. 2002 December; 88(6):1164-70), known to predispose horses to the disease of equine protozoal myelitis, we used the microarray to determine gene expression pattern signatures for stress. Detection of a stress profile that predisposes horses to disease could affect recommended treatments, such as immunostimulants or immunoprotectants, such as antibiotics.

Stress induces many changes in the neuroendocrine, immune, and hormonal systems that alters blood and tissue concentrations of corticosteroids, immunoglobulins, cytokines and other mediators of pathways associated with the fright- or -flight, inflammatory, and other body defense mechanisms. “Stress” is a relatively ill-defined syndrome, but one consequence of stress can be increased susceptibility to disease, presumably due to immunosuppression. One known initiator of stress in horses is shipping, such that respiratory sickness following shipping is so common as to receive a name called “shipping fever.” Some parameters, such as beta-endorphins, norepinephrine, corticosteroids, and pituitary hormones (ACTH) have been known to rise after shipping and other presumably stressful events such as exercise.

We examined large scale gene expression in blood cells of stressed and matched unstressed horses to identify an expression phenotype associated with stress. Twenty relatively unhandled yearling healthy horses, selected for inclusion in an equine protozoal study had stress induced by shipping the horses from Canada to Columbus, Ohio over an ˜16 hour time period. On arrival and prior to inoculation with sarcocystis to induce disease, 60 mLs of whole blood was drawn and placed in three 20-mL heparin tubes and shipped on ice, overnight to Dr Bertone's laboratory at The Ohio State University for processing. Stress was confirmed by the successful induced susceptibility to an infectious disease (Equine Protozoal Myelitis) in all of these horses. This confirmed a compromising stress state in these shipped horses. Five matched horses were identified in Ohio and had blood drawn at their home environment (no shipping) and blood processed in the same manner as the stressed horses.

Results:

On arrival of the blood in the laboratory, the buffy coat was withdrawn, snap frozen in liquid nitrogen, and frozen at −80° C. Buffy coats were systematically thawed and the RNA extracted.

The first protocol applied was the QIAamp® RNA Blood Mini Handbook for total RNA isolation from whole blood, which yielded moderate to poor RNA. These samples were not of sufficient quality or quantity to put on the microarray. The protocol was as follows:
1) Blood was centrifuged at 1200 rpm for 10 minutes.
2) Serum was drawn off and then the buffy coat was isolated.
3) Mixed 1 volume of blood with 5 volumes of Buffer EL in an appropriately sized tube.
4) Incubated for 10-15 minutes on ice. Mixed by vortexing briefly 2 times during incubation.
5) Centrifuged at 400×g for 10 minutes at 4° C., and completely removed and discarded the supernatant.
6) Added Buffer EL to the cell pellet (used 2 volumes of Buffer EL per volume of whole blood used in step 3). Resuspended cells by vortexing briefly.
7) Centrifuged at 400×g for 10 minutes at 400, and completely removed and discarded supernatant.

8) Added Buffer RLT to pelleted leukocytes according to the table below. Vortexed or pipetted to mix.

TABLE 24

| Buffer | Healthy whole | |
|---|---|---|
| RLT (μl) | blood (ml) | No. of leukocytes |
| 350 | Up to 0.5 | Up to 2 × 10⁶ |
| 600 | 0.5-1.5 | 2 × 10⁶ to 1 × 10⁷ |

9) Pipetted lysate directly into a QIAshredder spin column sitting in a 2-ml collection tube and centrifuged for 2 minutes at maximum speed to homogenize. Discarded QIAshredder spin column and saved homogenized lysate.
10) Added 1 volume (350 μl or 600 μl) of 70% ethanol to the homogenized lysate and mixed by pipetting.
11) Pipetted sample, including precipitate into new QIAamp spin column sitting in a 2 ml collection tube. Centrifuged for 15 seconds at ≧8,000×9.
12) Transferred the QIAamp spin column into a new 2-ml collection tube. Applied 700 μl Buffer RW1 to the QIAamp spin column and centrifuged for 15 seconds at ≧8,000×g to wash.
13) Placed QIAamp spin column in a new 2-ml collection tube. Pipetted 500 μl of buffer RPE into the QIAamp spin column and centrifuged for 15 seconds at ≧8,000×g.
14) Add 500% of Buffer RPE. Centrifuged at full speed for 3 minutes.
15) Placed the QIAamp spin column in a new 2 ml collection tube. Centrifuged at full speed for 1 minute.
16) Transferred the QIAamp spin column into a 1.5 ml microcentrifuge tube and pipeted 30-50 μl of Rnase-free water directly onto the QIAamp membrane. Centrifuged for 1 minute at ≧8,000×g to elute.

The second protocol used was the TRIzol® Bodily Fluids Protocol, which yielded moderate to good RNA. This resulted in sufficient quality and quantity of RNA from the five horses that were successfully processed on the microarray. The protocol was as follows:
1) Blood was centrifuged at 1200 rpm for 10 minutes.
2) Serum was drawn off and then the buffy coat was isolated.
3) HOMOGENIZATION
   The samples were homogenized with the addition of 0.75 ml TRIzol Reagent per 0.25 ml buffy coat.
4) PHASE SEPARATION
   Incubated the homogenized samples for 5 minutes at 15 to 30° C. Added 0.2 ml of chloroform per 1 ml of TRIzol Reagent Capped tubes securely. Shook tubes vigorously by hand for 15 seconds and incubated them at 15 to 30° C. for 2-3 minutes. Centrifuged samples at no more than 12,000× g for 15 minutes.
5) RNA PRECIPITATION
   Transferred the aqueous phase to a fresh tube. Precipitated the RNA from the aqueous phase by mixing with isopropyl alcohol. Used 0.5 ml of isopropyl alcohol per 1 ml of TRIzol Reagent used for the initial homogenization. Incubated the samples at 15 to 30° C. for 10 minutes and centrifuged at no more than 12,000×g for 10 minutes.
6) RNA WASH
   Removed the supernatant. Washed the RNA pellet once with 75% ethanol, adding at least 1 ml of 75% ethanol per 1 ml TRIzol Reagent used in the initial homogenization. Mixed the sample by vortexing and centrifuging at no more than 7,500×g for 5 minutes.
7) REDISSOLVING THE RNA
   At the end of the procedure, removed supernatant (leaving only the pellet) and briefly air dried the RNA pellet. The RNA pellets were redissolved in 30 μl of Rnase free water.

RNA from the top 5 samples in quantity and quality of RNA from stressed (n=5) and unstressed (n=4) were further processed for the study. Quality of RNA was further checked with a bioanalyzer (Agilent Technologies) and 1% agarose gels in a subset of samples. Horse and sample characteristics are listed in Table 25 below.

TABLE 25
Signalment and RNA characteristics of blood buffy coat used for the
microarray analysis.

| | | | | Age | | |
|---|---|---|---|---|---|---|
| Horse | Treatment | Sex | Breed | (yrs) | tRNA (μg) | 260/280 Ratio |
| 2B | Control | M | Belgian | 1 | 39 | 2.138 |
| 3C | Control | M | Belgian | 1 | 45.12 | 2.212 |
| 5C | Control | M | Belgian | 1 | 8.28 | 1.971 |
| 6B | Control | M | Belgian | 1 | 6.6 | 6.6 |
| 41A | Stressed | M | Belgian | 1 | 7.44 | 1.632 |
| 42B | Stressed | M | Belgian | 1 | 4.8 | 1.818 |
| 52B | Stressed | M | Belgian | 1 | 5.04 | 1.75 |
| 69A | Stressed | M | Belgian | 1 | 4.08 | 1.7 |
| 70A | Stressed | M | Belgian | 1 | 5.52 | 1.643 |

All protocols were conducted in accordance with the manufacturer's instructions. (Affymetrix, Inc.) Total RNA (5 ug) was reverse transcribed into double-stranded cDNA by use of a polymerase (Superscript II, Invitrogen) and the T7-(dT) 24 primer (Operon). Biotinylated cRNA was synthesized by in vitro transcription. The cRNA products were fragmented prior to hybridization overnight at 45° C. for 16 hours. Microarrays were washed at low- and high-stringent conditions and stained with streptavidin-phycoerythrin in accordance with an established protocol (EukGE-WS2).

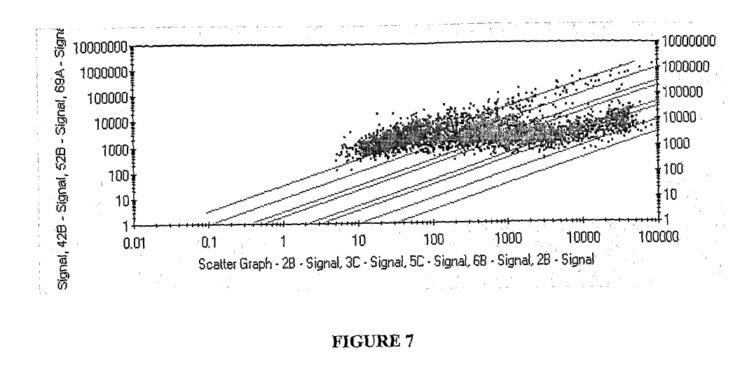

A cluster point graph of the combined data for expressed genes is shown in FIG. 7. Drift patterns are visually obvious showing a selection of genes that are upregulated in stress and a mass down regulation of gene expression in stressed horses. Data analysis was initially performed by use of a commercially available software package. (GCOS, Affymetrix, Inc.) Variables for performance of the microarray, such as signal intensity, were determined by use of statistical algorithms.

For the initial analysis from the Absolute CHP files, all Affymetrix control probes, and any probes which did not have at least 4 present calls among the 10 total chips were removed. For the remaining 2047 probe sets a t-test comparing unstressed control samples to stressed samples and a Bonferroni correction to adjust the p values for the number of multiple comparisons (2047) was performed. Fifteen probe sets had significant changes in gene expression and represent a statistically significant signature for stress. See Table 26.

TABLE 26

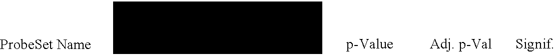

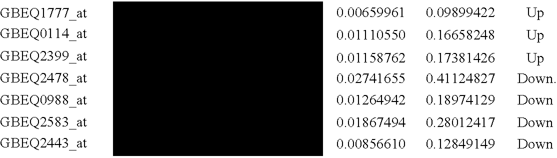

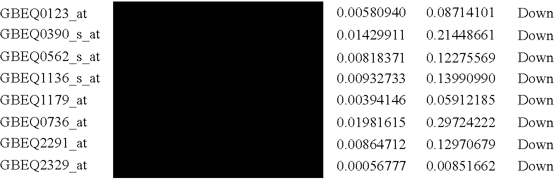

Further analysis of the Comparative CHP files evaluates the count of number of chips for each of the call changes for each gene (Increased, Decreased, and No Change) made by the Affymetrix software. This corresponds to the number of possible comparisons of the stressed microarrays (5 arrays) to the unstressed microarrays (4 arrays), or 20 comparisons for this study. These probe sets were not filtered. Stressed was compared as a ratio to control—one sorted for decreases and the other for increases. Considering that 16 out of 20 chips (80% agreement) a reliable change, then there were 60 increased and 150 decreased genes that may be biologically significant based on probability. In Table 27 below, of 20 total gene chip comparisons, i.e., experimental (stressed) to control (unstressed), there was 1 gene that was always increased in every stressed to unstressed comparison, 7 genes were increased in 95% of the stressed to unstressed comparison, 15 genes were increased in 90% of the stressed to unstressed comparison, 13 genes were increased in 85% of the stressed to unstressed comparison and 24 genes were increased in 80% of the stressed to unstressed comparison. The addition of subsets of these genes to the gene signature in sets of 10 would improve the accuracy of identifying stress in horses.

TABLE 27

| | | Total | | Marg | |
|---|---|---|---|---|---|
| ProbeSetName | Ratio | comparisons | I | Inc | No Change |
| GBEQ2890_at | Exp/Ctl | 20 | 20 | | |
| GBEQ2817_at | Exp/Ctl | 20 | 19 | | |
| GBEQ0693_at | Exp/Ctl | 20 | 19 | | 1 |
| GBEQ2366_at | Exp/Ctl | 20 | 19 | | 1 |
| GBEQ2697_s_at | Exp/Ctl | 20 | 19 | | 1 |
| GBEQ2730_at | Exp/Ctl | 20 | 19 | | 1 |
| GBEQ3018_x_at | Exp/Ctl | 20 | 19 | | 1 |
| GBEQ3187_at | Exp/Ctl | 20 | 19 | | 1 |
| GBCA0302_at | Exp/Ctl | 20 | 18 | | 2 |
| GBCA0390_at | Exp/Ctl | 20 | 18 | 1 | 1 |
| GBEQ1830_at | Exp/Ctl | 20 | 18 | 1 | 1 |
| GBEQ1930_at | Exp/Ctl | 20 | 18 | | 2 |
| GBEQ1989_at | Exp/Ctl | 20 | 18 | | 2 |
| GBEQ2216_at | Exp/Ctl | 20 | 18 | | 2 |
| GBEQ2328_at | Exp/Ctl | 20 | 18 | | 2 |
| GBEQ2392_at | Exp/Ctl | 20 | 18 | | 2 |
| GBEQ2738_at | Exp/Ctl | 20 | 18 | | 2 |
| GBEQ2897_at | Exp/Ctl | 20 | 18 | | 2 |
| GBEQ2967_at | Exp/Ctl | 20 | 18 | | 2 |
| GBEQ3034_at | Exp/Ctl | 20 | 18 | 1 | 1 |
| GBEQ3069_at | Exp/Ctl | 20 | 18 | | 2 |
| GBEQ3095_at | Exp/Ctl | 20 | 18 | | 2 |
| GBEQ3162_at | Exp/Ctl | 20 | 18 | | 2 |
| GBCA0066_at | Exp/Ctl | 20 | 17 | | 3 |
| GBCA0119_at | Exp/Ctl | 20 | 17 | | 3 |
| GBCA0149_at | Exp/Ctl | 20 | 17 | | 3 |
| GBCA0255_at | Exp/Ctl | 20 | 17 | | 3 |
| GBEQ0001-5_s_at | Exp/Ctl | 20 | 17 | | 3 |
| GBEQ0042_at | Exp/Ctl | 20 | 17 | 1 | 2 |
| GBEQ0058_at | Exp/Ctl | 20 | 17 | | 3 |
| GBEQ0208_at | Exp/Ctl | 20 | 17 | | 3 |
| GBEQ0924_at | Exp/Ctl | 20 | 17 | | 3 |
| GBEQ3077_at | Exp/Ctl | 20 | 17 | | 3 |
| GBEQ3145_at | Exp/Ctl | 20 | 17 | | 3 |
| GBEQ3172_at | Exp/Ctl | 20 | 17 | 1 | 2 |
| GBEQ3217_at | Exp/Ctl | 20 | 17 | 1 | 2 |
| GBCA0141_at | Exp/Ctl | 20 | 16 | | 4 |
| GBCA0154_at | Exp/Ctl | 20 | 16 | | 4 |
| GBCA0199_at | Exp/Ctl | 20 | 16 | | 4 |
| GBCA0284_at | Exp/Ctl | 20 | 16 | 1 | 3 |
| GBCA0462_at | Exp/Ctl | 20 | 16 | 1 | 3 |
| GBEQ0011_at | Exp/Ctl | 20 | 16 | 1 | 3 |
| GBEQ0036_at | Exp/Ctl | 20 | 16 | | 4 |
| GBEQ0145_at | Exp/Ctl | 20 | 16 | | 4 |
| GBEQ0153_at | Exp/Ctl | 20 | 16 | | 4 |
| GBEQ0205_at | Exp/Ctl | 20 | 16 | | 4 |
| GBEQ0210_at | Exp/Ctl | 20 | 16 | | 4 |
| GBEQ0310_at | Exp/Ctl | 20 | 16 | | 4 |
| GBEQ0391_at | Exp/Ctl | 20 | 16 | 1 | 3 |
| GBEQ1665_at | Exp/Ctl | 20 | 16 | 1 | 3 |
| GBEQ2088_s_at | Exp/Ctl | 20 | 16 | | 4 |
| GBEQ2327_at | Exp/Ctl | 20 | 16 | 1 | 3 |
| GBEQ2801_at | Exp/Ctl | 20 | 16 | | 4 |
| GBEQ2816_at | Exp/Ctl | 20 | 16 | | 4 |
| GBEQ2891_at | Exp/Ctl | 20 | 16 | | 4 |
| GBEQ2911_at | Exp/Ctl | 20 | 16 | | 4 |
| GBEQ3038_at | Exp/Ctl | 20 | 16 | | 4 |
| GBEQ3085_at | Exp/Ctl | 20 | 16 | | 4 |
| GBEV0062_at | Exp/Ctl | 20 | 16 | | 4 |

In Table 28 below, of 20 total gene chip comparisons, i.e., experimental (stressed) to control (unstressed), there was 1 gene that was always increased in every stressed to unstressed comparison, 7 genes were increased in 95% of the stressed to unstressed comparison, 15 genes were increased in 90% of the stressed to unstressed comparison, 13 genes were increased in 85% of the stressed to unstressed comparison and 24 genes were increased in 80% of the stressed to unstressed comparison. The addition of subsets of these genes to the gene signature in sets of 10 would improve the accuracy of identifying stress in horses.

TABLE 28

| ProbeSetName | Ratio | Total Comparisons | Decreased | MarDecr | Nn Change |
|---|---|---|---|---|---|
| GBEQ0048-3_at | Exp/Ctl | 20 | 20 | | |
| GBEQ0123_at | Exp/Ctl | 20 | 20 | | |
| GBEQ0296_at | Exp/Ctl | 20 | 20 | | |
| GBEQ0330_at | Exp/Ctl | 20 | 20 | | |
| GBEQ0355_at | Exp/Ctl | 20 | 20 | | |
| GBEQ0390_s_at | Exp/Ctl | 20 | 20 | | |
| GBEQ0501_at | Exp/Ctl | 20 | 20 | | |
| GBEQ0634_s_at | Exp/Ctl | 20 | 20 | | |
| GBEQ0736_at | Exp/Ctl | 20 | 20 | | |
| GBEQ0820_at | Exp/Ctl | 20 | 20 | | |
| GBEQ0894_at | Exp/Ctl | 20 | 20 | | |
| GBEQ0980_at | Exp/Ctl | 20 | 20 | | |
| GBEQ1044_at | Exp/Ctl | 20 | 20 | | |
| GBEQ1071_at | Exp/Ctl | 20 | 20 | | |
| GBEQ1165_at | Exp/Ctl | 20 | 20 | | |
| GBEQ1179_at | Exp/Ctl | 20 | 20 | | |
| GBEQ1207_at | Exp/Ctl | 20 | 20 | | |
| GBEQ1245_at | Exp/Ctl | 20 | 20 | | |
| GBEQ1310_at | Exp/Ctl | 20 | 20 | | |
| GBEQ1327_at | Exp/Ctl | 20 | 20 | | |
| GBEQ1330_at | Exp/Ctl | 20 | 20 | | |
| GBEQ1387_at | Exp/Ctl | 20 | 20 | | |
| GBEQ1454_at | Exp/Ctl | 20 | 20 | | |
| GBEQ1503_at | Exp/Ctl | 20 | 20 | | |
| GBEQ1634_at | Exp/Ctl | 20 | 20 | | |
| GBEQ1706_at | Exp/Ctl | 20 | 20 | | |
| GBEQ1771_at | Exp/Ctl | 20 | 20 | | |
| GBEQ1788_at | Exp/Ctl | 20 | 20 | | |
| GBEQ1813_at | Exp/Ctl | 20 | 20 | | |
| GBEQ1814_at | Exp/Ctl | 20 | 20 | | |
| GBEQ1836_at | Exp/Ctl | 20 | 20 | | |
| GBEQ1912_s_at | Exp/Ctl | 20 | 20 | | |
| GBEQ1993_at | Exp/Ctl | 20 | 20 | | |
| GBEQ1997_s_at | Exp/Ctl | 20 | 20 | | |
| GBEQ2202_at | Exp/Ctl | 20 | 20 | | |
| GBEQ2226_at | Exp/Ctl | 20 | 20 | | |
| GBEQ2238_at | Exp/Ctl | 20 | 20 | | |
| GBEQ2291_at | Exp/Ctl | 20 | 20 | | |
| GBEQ2329_at | Exp/Ctl | 20 | 20 | | |
| GBEQ2372_at | Exp/Ctl | 20 | 20 | | |
| GBEQ2452_at | Exp/Ctl | 20 | 20 | | |
| GBEQ2576_at | Exp/Ctl | 20 | 20 | | |
| GBEQ2752_at | Exp/Ctl | 20 | 20 | | |
| GBEQ0562_s_at | Exp/Ctl | 20 | 19 | | 1 |
| GBEQ0659_at | Exp/Ctl | 20 | 19 | | 1 |
| GBEQ0685_at | Exp/Ctl | 20 | 19 | 1 | |
| GBEQ0872_at | Exp/Ctl | 20 | 19 | 1 | |
| GBEQ0938_at | Exp/Ctl | 20 | 19 | | 1 |
| GBEQ1176_at | Exp/Ctl | 20 | 19 | | 1 |
| GBEQ1205_at | Exp/Ctl | 20 | 19 | | 1 |
| GBEQ1266_s_at | Exp/Ctl | 20 | 19 | | 1 |
| GBEQ1298_at | Exp/Ctl | 20 | 19 | | 1 |
| GBEQ1358_at | Exp/Ctl | 20 | 19 | 1 | |
| GBEQ1438_at | Exp/Ctl | 20 | 19 | | 1 |
| GBEQ1495_at | Exp/Ctl | 20 | 19 | | 1 |
| GBEQ1588_at | Exp/Ctl | 20 | 19 | | 1 |
| GBEQ1916_at | Exp/Ctl | 20 | 19 | | 1 |
| GBEQ1988_at | Exp/Ctl | 20 | 19 | | 1 |
| GBEQ2000_at | Exp/Ctl | 20 | 19 | | 1 |
| GBEQ2173_s_at | Exp/Ctl | 20 | 19 | | 1 |
| GBEQ2227_at | Exp/Ctl | 20 | 19 | | 1 |
| GBEQ2294_at | Exp/Ctl | 20 | 19 | | 1 |
| GBEQ2481_at | Exp/Ctl | 20 | 19 | | 1 |
| GBEQ2637_at | Exp/Ctl | 20 | 19 | | 1 |
| GBEQ2767_at | Exp/Ctl | 20 | 19 | | 1 |
| GBEQ2895_at | Exp/Ctl | 20 | 19 | | 1 |
| GBEQ3079_at | Exp/Ctl | 20 | 19 | | 1 |
| GBEQ3218_at | Exp/Ctl | 20 | 19 | | 1 |
| GBEQ0056_s_at | Exp/Ctl | 20 | 18 | | 2 |
| GBEQ0395_at | Exp/Ctl | 20 | 18 | | 2 |
| GBEQ0440_s_at | Exp/Ctl | 20 | 18 | 1 | 1 |
| GBEQ0448_s_at | Exp/Ctl | 20 | 18 | | 2 |
| GBEQ0516_at | Exp/Ctl | 20 | 18 | | 2 |
| GBEQ0578_at | Exp/Ctl | 20 | 18 | 1 | 1 |
| GBEQ0694_s_at | Exp/Ctl | 20 | 18 | | 2 |
| GBEQ0862_s_at | Exp/Ctl | 20 | 18 | | 2 |
| GBEQ0887_s_at | Exp/Ctl | 20 | 18 | 1 | 1 |
| GBEQ1275_at | Exp/Ctl | 20 | 18 | | 2 |
| GBEQ1360_at | Exp/Ctl | 20 | 18 | | 2 |
| GBEQ1395_at | Exp/Ctl | 20 | 18 | | 2 |
| GBEQ1457_at | Exp/Ctl | 20 | 18 | | 2 |
| GBEQ1582_at | Exp/Ctl | 20 | 18 | | 2 |
| GBEQ1609_at | Exp/Ctl | 20 | 18 | | 2 |
| GBEQ2041_at | Exp/Ctl | 20 | 18 | | 2 |
| GBEQ2063_at | Exp/Ctl | 20 | 18 | | 2 |
| GBEQ2284_at | Exp/Ctl | 20 | 18 | | 2 |
| GBEQ2338_at | Exp/Ctl | 20 | 18 | | 2 |
| GBEQ2406_at | Exp/Ctl | 20 | 18 | | 2 |
| GBEQ2437_at | Exp/Ctl | 20 | 18 | | 2 |
| GBEQ2483_at | Exp/Ctl | 20 | 18 | | 2 |
| GBEQ2583_at | Exp/Ctl | 20 | 18 | | 2 |
| GBEQ2632_at | Exp/Ctl | 20 | 18 | 1 | 1 |
| GBEQ2671_at | Exp/Ctl | 20 | 18 | | 2 |
| GBEQ0048-5_at | Exp/Ctl | 20 | 17 | | 3 |
| GBEQ0531_at | Exp/Ctl | 20 | 17 | | 3 |
| GBEQ0576_at | Exp/Ctl | 20 | 17 | 1 | 2 |
| GBEQ0632_x_at | Exp/Ctl | 20 | 17 | | 3 |
| GBEQ0877_s_at | Exp/Ctl | 20 | 17 | | 3 |
| GBEQ0947_s_at | Exp/Ctl | 20 | 17 | | 3 |
| GBEQ0997_at | Exp/Ctl | 20 | 17 | | 3 |
| GBEQ1136_s_at | Exp/Ctl | 20 | 17 | 1 | 2 |
| GBEQ1144_at | Exp/Ctl | 20 | 17 | | 3 |
| GBEQ1168_s_at | Exp/Ctl | 20 | 17 | 1 | 2 |
| GBEQ1426_at | Exp/Ctl | 20 | 17 | | 3 |
| GBEQ1631_at | Exp/Ctl | 20 | 17 | 1 | 2 |
| GBEQ1662_at | Exp/Ctl | 20 | 17 | | 3 |
| GBEQ1881_at | Exp/Ctl | 20 | 17 | 1 | 2 |
| GBEQ1914_at | Exp/Ctl | 20 | 17 | | 3 |
| GBEQ1977_at | Exp/Ctl | 20 | 17 | | 3 |
| GBEQ2265_at | Exp/Ctl | 20 | 17 | 2 | 1 |
| GBEQ2318_at | Exp/Ctl | 20 | 17 | 1 | 2 |
| GBEQ2341_s_at | Exp/Ctl | 20 | 17 | | 3 |
| GBEQ2616_at | Exp/Ctl | 20 | 17 | | 3 |
| GBEQ2646_at | Exp/Ctl | 20 | 17 | | 3 |
| GBEQ3002_at | Exp/Ctl | 20 | 17 | | 3 |
| GBEQ0650_s_at | Exp/Ctl | 20 | 16 | | 3 |
| GBEQ1249_s_at | Exp/Ctl | 20 | 16 | | 3 |
| GBEQ2288_at | Exp/Ctl | 20 | 16 | | 3 |
| GBEQ0527_at | Exp/Ctl | 20 | 16 | | 4 |
| GBEQ0618_s_at | Exp/Ctl | 20 | 16 | | 4 |
| GBEQ0728_at | Exp/Ctl | 20 | 16 | | 4 |
| GBEQ0824_at | Exp/Ctl | 20 | 16 | | 4 |
| GBEQ0886_s_at | Exp/Ctl | 20 | 16 | | 4 |
| GBEQ1070_at | Exp/Ctl | 20 | 16 | | 4 |
| GBEQ1107_at | Exp/Ctl | 20 | 16 | | 4 |
| GBEQ1124_at | Exp/Ctl | 20 | 16 | | 4 |
| GBEQ1151_at | Exp/Ctl | 20 | 16 | | 4 |
| GBEQ1263_s_at | Exp/Ctl | 20 | 16 | 1 | 3 |
| GBEQ1566_at | Exp/Ctl | 20 | 16 | | 4 |
| GBEQ1568_at | Exp/Ctl | 20 | 16 | 1 | 3 |
| GBEQ1630_at | Exp/Ctl | 20 | 16 | 1 | 3 |
| GBEQ1638_at | Exp/Ctl | 20 | 16 | | 4 |
| GBEQ1686_at | Exp/Ctl | 20 | 16 | | 4 |
| GBEQ1694_at | Exp/Ctl | 20 | 16 | | 4 |
| GBEQ1762_at | Exp/Ctl | 20 | 16 | | 4 |
| GBEQ1792_s_at | Exp/Ctl | 20 | 16 | | 4 |
| GBEQ1809_at | Exp/Ctl | 20 | 16 | | 4 |
| G8EQ1832_at | Exp/Ctl | 20 | 16 | | 4 |
| GBEQ1876_at | Exp/Ctl | 20 | 16 | 1 | 3 |
| GBEQ1969_at | Exp/Ctl | 20 | 16 | | 4 |
| GBEQ2115_at | Exp/Ctl | 20 | 16 | 1 | 3 |
| GBEQ2153_at | Exp/Ctl | 20 | 16 | 2 | 2 |
| GBEQ2334_s_at | Exp/Ctl | 20 | 16 | | 4 |
| GBEQ2368_at | Exp/Ctl | 20 | 16 | | 4 |
| GBEQ2455_s_at | Exp/Ctl | 20 | 16 | 1 | 3 |
| GBEQ2511_at | Exp/Ctl | 20 | 16 | | 4 |
| GBEQ2655_at | Exp/Ctl | 20 | 16 | | 4 |
| GBEQ2973_at | Exp/Ctl | 20 | 16 | | 4 |
| GBEQ3020_at | Exp/Ctl | 20 | 16 | | 4 |
| GBEQ3099_at | Exp/Ctl | 20 | 16 | | 4 |

### Example 12

### Gene Expression Patterns to Detect Laminitis in Horses

Laminitis is a major cause of lameness in both cattle and horses resulting in loss of use and production in both species. The disease is characterized by the loss of the laminar structure within the hoof wall of horses and cattle. This destruction leaves the coffin bone without support, causing rotation and sinking of the bone within the hoof. Once the disease has begun, it can lead to a chronic debilitating lameness of which there is little that can be done. Although the disease is common, not much is known of its etiology and to date there are no therapies available for treatment or prevention of the disease. Currently, there are three theories on the pathogenesis of laminitis; the metabolic/toxic hypothesis, the vascular/ischemia, and the inflammatory hypothesis. This Example seeks to examine the role of inflammatory cytokines on the pathogenesis of equine laminitis.

Central inflammatory cytokines, such as, are highly expressed by monocytes and macrophages after infection, tissue damage and during systemic inflammation. Proinflammatory cytokines, IL-1 and TNF, have numerous overlapping biological functions such as inducing other inflammatory cytokines. Microarray studies on human endothelial cells have shown 25 out of 66 genes are up-regulated in common by both IL-1 and TNF inflammatory cytokines. Some of the genes expressed by both IL-1 and TNF include, but are not limited to, chemokines, matrix metalloproteinase, inflammatory cytokines, signal transduction proteins, and metabolic proteins. Previous attempts at blocking systemic inflammation using IL-1 or TNF receptor antagonists and soluble receptors have proven in most cases to be ineffectual. This failure to produce a biological effect maybe due to the degree of overlapping between these two cytokines, not the effectiveness of the individual blocking methods. It is commonly observed that clinical laminitis is closely associated with systemic inflammatory disease, sepsis, and endotoxemia.

Normal horses euthanized for unrelated reasons had digital vessels freshly removed and the endothelium stripped from the inside surface. Clinical cases of horses with naturally occurring laminitis that were euthanized in the acute phase of the disease (<72 hours of clinical signs), had similar tissue harvest. The tissue was homogenized and RNA extracted and processed on the microarray in the same manner as described in Example 11.

Specifically, genes identified as up- and down-regulated in laminitis and representing potential markers of laminitis are graphically represented in the cluster diagram below and are listed in Table 29 below.

TABLE 29
Genes that are up regulated 3-fold or down regulated 5-fold in
laminitis endothelium and represent a profile of gene
expression for laminitis

| | Fold Change |
|---|---|
| GBEQ3087_at | 8.6 |
| GBEQ0825_at | 8 |
| GBEQ2750_at | 7 |
| GBEQ0866_at | 7 |
| GBEQ2948_at | 6.1 |
| GBEQ1467_at | 5.7 |
| GBEQ1051_at | 5.7 |
| GBEQ1389_at | 5.3 |
| GBEQ3145_at | 4.9 |
| GBEQ1198_at | 4.9 |
| GBEQ1163_at | 4.9 |
| GBEQ1299_at | 4.6 |
| GBEQ2385_s_at | 4 |
| GBEQ1326_at | 4 |
| GBEQ1888_at | 4 |
| GBEQ0487_at | 3.7 |
| GBEQ3076_at | 3.7 |
| GBEQ2567_at | 3.7 |
| GBEQ2051_at | 3.7 |
| GBEQ2277_at | 3.7 |
| GBEQ2605_at | 3.7 |
| GBEQ2344_at | 3.5 |
| GBEQ2893_at | 3.5 |
| GBEQ1287_at | 3.5 |
| GBEQ0636_at | 3.5 |
| GBEQ1489_at | 3.5 |
| GBEQ0744_at | 3.5 |
| GBEQ1324_at | 3.5 |
| GBEQ2070_at | 3.3 |
| GBEQ3144_at | 3.3 |
| GBEQ1861_at | 3.3 |
| GBEQ0400_at | 3.3 |
| GBEQ0304_at | 3.3 |
| GBEQ0616_at | 3.3 |
| GBEQ1774_at | 3.3 |
| GBEQ1166_at | 3.3 |
| GBEQ2381_at | 3.3 |
| GBEQ1178_at | 3.3 |
| GBEQ0863_at | 3.3 |
| GBEQ1347_at | 3.3 |
| GBEQ2002_at | 3 |
| GBEQ0979_at | 3 |
| GBEQ0660_at | 3 |
| GBEQ2132_at | 3 |
| GBEQ3104_at | 3 |
| GBEQ1442_at | 3 |
| GBEQ2784_at | 3 |
| GBEQ2450_at | 3 |
| GBEQ0560_at | 3 |
| GBEQ2982_at | 3 |
| GBEQ0477_s_at | −5.3 |
| GBEQ0279_at | −5.3 |
| GBEQ1778_at | −5.3 |
| GBEQ0115_at | −5.3 |
| GBEQ1405_at | −5.3 |
| GBEQ2501_at | −5.3 |
| GBEQ2261_at | −5.3 |
| GBEQ2786_at | −5.3 |
| GBEQ1738_at | −5.7 |
| GBEQ1564_at | −5.7 |
| GBEQ2420_at | −5.7 |
| GBEQ2534_at | −5.7 |
| GBEQ2099_at | −5.7 |
| GBEQ2186_at | −5.7 |
| GBEQ1444_at | −5.7 |
| GBEQ0255_at | −6.1 |
| GBEQ1239_at | −6.1 |
| GBEQ0067_at | −6.1 |
| GBEQ1254_at | −6.1 |
| GBEQ1903_at | −6.1 |
| G8EQ2687_at | −6.5 |
| GBEQ0701_at | −6.5 |
| GBEQ0433_at | −6.5 |
| GBEQ0255_x_at | −7 |
| GBEQ0255_s_at | −7.5 |
| GBEQ0977_at | −7.5 |
| GBEQ1014_at | −7.5 |
| GBEQ1469_at | −8 |
| GBEQ2212_at | −8.6 |
| GBEQ1722_at | −8.6 |
| GBEQ1497_at | −9.2 |
| GBEQ2548_at | −9.2 |
| GBEQ2700_s_at | −9.8 |
| GBEQ0238_s_at | −9.8 |
| GBEQ1911_at | −11.3 |
| GBEQ0047_at | −13.92 |
| GBEQ0281_at | −13.9 |
| GBEQ0275_at | −22.6 |

### Example 13

### Construction of Canine Nucleic Acid Database and Microarray

Example 1 was repeated to create a canine database, with some alterations made in the procedure. First, due to the limitation of the microarray size and the very large number of canine sequences publicly available, only the fully annotated 3′-complete mRNA canine coding sequences were selected. No canine ESTs were included in the processing. Otherwise, the steps were similar as in Example 1: GetCanine->GetCDS->CheckmRNA->GetThreePrimeCompleteCDS->FastaG->ClusterG.

### Example 14

### Use of the Canine Microarray and Gene Expression Patterns to Detect Osteoarthritis in Dogs

Articular cartilage was harvested from freshly removed osteoarthritic hip joints at joint replacement surgery and compared to age and size matched, freshly euthanized normal dogs from the humane society. Cartilage was digested in 0.2% collagenase to release the chondrocytes. Cells were allowed to grow in medium for 3 days before harvested for RNA extraction. RNA extraction was performed in the same manner as described for equine synovial cells in Example 3 and processed on the microarray. Data analysis was performed by use of commercially available software packages (Microarray suite 5.0, Affymetrix Inc, Santa Clara, Calif.; MicroDB, Affymetrix Inc, Santa Clara, Calif.; Data Mining Tool 3.0, Affymetrix Inc, Santa Clara, Calif.).

Expression of genes on the array was excellent for cartilage genes, approximately 47% with mean signal intensities of ˜4,000 (See Table 30 below).

TABLE 30

| | | | Present | | Absent | | Marginal | |
|---|---|---|---|---|---|---|---|---|
| | Detection call | | Mean | | Mean | | Mean | |
| | Signal | Intensity | signal | #genes | signal | #genes | signal | #genes |
| Dog | Mean | Maximum | intensity | (%) | intensity | (%) | intensity | (%) |
| Control | 1997 | 35235 | 4240 | 254 | 85 | 299 | 488 | 9 |
| | | | | (45) | | (53) | | (2) |
| OA | 1947 | 30851 | 3910 | 273 | 85 | 275 | 226 | 14 |
| | | | | (49) | | (49) | | (2) |

Use of this microarray on canine tissue samples has identified genes important in canine osteoarthritis (OA). Table 31 below generally shows how genes are up- and down-regulated in osteoarthritis.

TABLE 31

| Genes Changed | |
|---|---|
| # Up- | # Down- |
| Regulated | Regulated |

| Group | Total # | >2-fold | Total # | >2-fold |
|---|---|---|---|---|
| Control | — | — | — | — |
| OA | 56 | 25 | 52 | 11 |

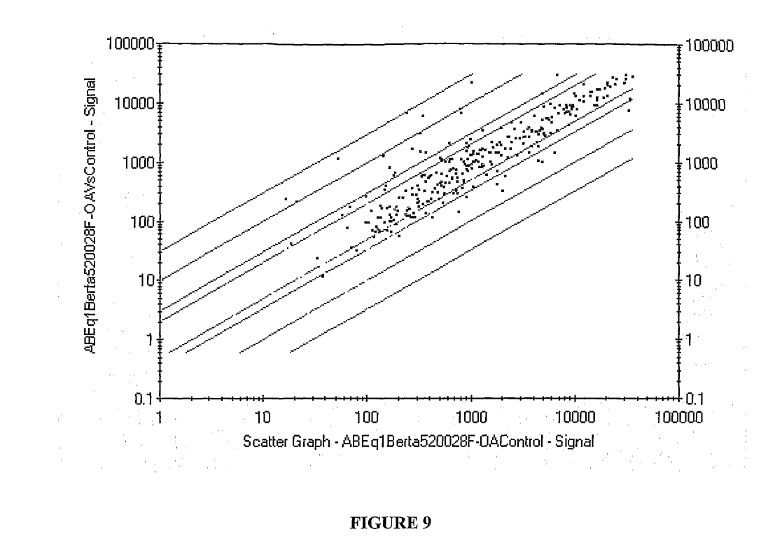

Specifically, genes identified as up and down regulated in OA and representing markers of OA are graphically represented in the cluster diagram shown in FIG. 9 and are listed in Table 32 below.

TABLE 32
Up-regulated and down-regulated genes in OA dog

| | Fold- | |
|---|---|---|
| Accession no | Change | Description |
| U12234 | 30 | Canis familiaris interleukin-6 (IL-6) |
| | | mRNA, complete cds. |
| U32086 | 26 | Canis familiaris vascular cell adhesion |
| | | molecule-1 mRNA, complete cds. |
| U29653 | 26 | Canis familiaris monocyte |
| | | chemoattractant protein-1 mRNA, |
| | | complete cds. |
| L23087 | 12 | Canis familiaris E-selectin mRNA, |
| | | complete cds. |
| AB098562 | 11 | Canis familiaris RANTES mRNA for |
| | | RANTES protein, complete cds. |
| AB054642 | 10 | Canis familiaris mRNA for chemokine, |
| | | complete cds. |
| AY262732 | 10 | Canis familiaris 18S ribosomal RNA |
| | | gene, partial sequence. |
| U10308 | 9 | Canis familiaris interleukin-8 mRNA, |
| | | complete cds. |
| D84397 | 6.5 | Canis familiaris mRNA for |
| | | metallothionein-1, complete cds. |
| AF117714 | 5 | Canis familiaris hematopoietic antigen |
| | | CD38 mRNA, complete cds. |
| AF077821 | 4.3 | Canis familiaris inducible nitric oxide |
| | | synthase mRNA, complete cds. |
| AF177217 | 4 | Canis familiaris matrix |
| | | metalloproteinase-2 (MMP-2) mRNA, |
| | | partial cds. |
| X92505 | 3.5 | C. familiaris mRNA for VIP17/MAL |
| | | proteolipid. |
| S49738 | 3 | Granulocyte-macrophage colony- |
| | | stimulating factor (dogs, mRNA, 809 nt). |
| AF077817 | 3 | Canis familiaris tissue inhibitor of |
| | | metalloproteinases TIMP-1 mRNA, |
| | | complete cds. |
| S42999 | 2.6 | K-ras (dogs, spleen, mRNA Partial, 212 nt). |
| Proprietary | 2.6 | LIB4005-007-Q6-K1-A6 |
| AB043896 | 2.6 | Canis familiaris mRNA for Rad51, |
| | | complete cds. |
| AF177934 | 2.5 | Canis familiaris prostaglandin E2 |
| | | receptor EP4 subtype mRNA, complete |
| | | cds. |
| AY044905 | 2.3 | Canis familiaris prostaglandin G/H |
| | | synthase-2 mRNA, complete cds. |
| X05297 | 2.3 | Dog kidney mRNA for (Na+/K+)- |
| | | ATPase beta-subunit. |
| Proprietary | 2.3 | LIB4217-040-R1-K1-G8 |
| AY057077 | 2.1 | Canis familiaris thiopurine |
| | | methyltransferase (TPMT) mRNA, |
| | | complete cds, alternatively spliced. |
| AJ388535 | 2.1 | Canis familiaris mRNA for partial |
| | | ubiquitin carrier protein (E2-EPF gene). |
| AF212974 | 2.1 | Canis familiaris gamma tubulin (TUBG) |
| | | mRNA, complete cds. |
| AF023169 | −4.3 | Canis familiaris type IIA procollagen |
| | | mRNA, complete cds. |
| U65989 | −4.3 | Canis familiaris articular cartilage |
| | | aggrecan precursor, mRNA, complete |
| | | cds. |
| AF045773 | −4 | Canis familiaris adrenomedullin |
| | | precursor, mRNA, complete cds. |
| AF525493 | −3.5 | Canis familiaris H11 kinase mRNA, |
| | | complete cds. |
| U83140 | −3.5 | Canis familiaris biglycan mRNA, |
| | | complete cds. |
| AF525129 | −3.2 | Canis familiaris protein phosphatase |
| | | type 1 beta isoform mRNA, complete |
| | | cds. |
| AF535138 | −2.5 | Canis familiaris cyclooxygenase mRNA, |
| | | complete cds. |
| M35520 | −2.3 | C. familiaris GTP-binding protein (rab5) |
| | | mRNA, complete cds. |
| Proprietary | −2.3 | LIB4003-010-Q6-K1-H11 |
| AB075027 | −2.1 | Canis familiaris hsp70 mRNA for heat |
| | | shock protein 70, complete cds. |
| AF133250 | −2.1 | Canis familiaris vascular endothelial |
| | | growth factor 188 (VEGF) mRNA, |
| | | complete cds. |

### Example 15

### Canine Microarray Gene Expression Analysis for Molecular Therapy of Hip Disease

Dogs are human's best friends. There are about 300 different dog breeds in the world as a result of a long history of gene pool selection and mixing. The modern domestic dog is unique for the study of human genetic diseases in that it has a larger pedigree than that of the small, outbred human families. Moreover, many of the ˜360 known canine genetic diseases are homologs of the human disorders, including osteoarthritis secondary to hip dysplasia. These genetically complicated disorders are not fully controlled by a single gene and are suited for large-scale gene expression profiling to gain insight into the cross-talk associated with the abnormal phenotype. The use of microarrays for gene expression studies and diagnostics is becoming well established. The use of a species-specific microarray is of critical importance for accurate biomarker identification and monitoring of highly specific markers. In cross-species hybridization on microarrays, even single nucleotide mismatches can alter the detectable gene expression and relative intensities resulting in erroneous conclusions.

Canine disease gene cloning and characterization is the major limiting step in understanding the canine diseases at the gene level. In our preliminary analyses, the current public nucleotide database (GenBank) has stored close to 2 million canine related genetic records, while only 0.1% have been annotated with genetic function. Most nucleotide entries are unknown chromosomal sequences and expressed sequence tags of unknown function. With the maturation of primarily the human and mouse databases, tens of thousands of gene sequences have been functionally identified and mapped. Such information can be used to decipher the canine sequences through comparative analysis.

In this example, we describe the design and use of a canine database, similar to that described for equine in Example 1. The design annotates sequences by Blast to a human/mouse coding sequence database, trims for high quality sequence, substantially reduces duplication, and selects for 3′ complete sequencing to permit high resolution probe design critical for ribonucleic acid (RNA) detection by current technology that involves 3′ amplification.

Osteoarthritis (OA) is a debilitating disease affecting both canine and human patients. It is one of the most common sources of chronic pain treated by veterinarians, estimated to affect one in five of 68 million adult dogs and commonly affects the hip joint secondary to hip dysplasia. Accordingly, the incidence of musculoskeletal pathology in dogs less than one year of age has been estimated at 22%, often related to hip dysplasia. Use of large-scale gene expression profiling of osteoarthritic cartilage to assess phenotype and alterations with experimental manipulation are beginning to appear in the literature, including IL-1.

This example describes the generation of an exhaustive canine database for gene expression and applies this information to large-scale microarray analysis to assess the ability of molecular therapy to promote a regenerative phenotype in canine osteoarthritic (OA) cartilage. This example captures current state of the art technology made possible from the recent canine genome sequencing projects for both public academic use and the use in profiling inducible cellular dedifferentiation pathways of OA chondrocytes.

The current >1.5 million canine sequences on the public database will likely condense to <40,000 high quality, unique annotated canine sequences most of which will contain the criteria necessary for inclusion on a microarray, such as 3′-bias, and also, the bone morphogenetic protein-2 (BMP-2) in combination with interleukin-1 receptor antagonist (IL-1 ra) will induce gene expression patterns involving hundreds of genes that profile a healthier chondrocyte phenotype, including aggrecan and type II collagen up-regulation and metalloproteinase down-regulation.

This example describes the curation, pruning, and annotation of the public canine nucleotide database so it can be used for further canine genomic functional analysis or for generating canine species-specific large-scale gene expression microarrays. These data may complement the recent commercial canine high-density microarrays (Affymetrix), and allow for comparison of gene expression patterns of OA hip cartilage from dysplasic dogs that have been genetically engineered to express BMP-2 and/or IL-1ra as a measure of an induced de-differentiation gene expression profile typical of more healthy chondrocytes. This example proves initial efficacy of novel molecular therapies for hip dysplasia that can be delivered by joint injection, offering a pain-relieving and disease-modifying therapy.

The approach used to obtain the equine database was through queries to NCBI, and downloaded the result to the desktop computer. For equine sequences (˜20,000 records), this is acceptable. However, for dog, it may be difficult to download ˜2 million records in GenBank format from the web to the local computer (PC) by query. Thus, for canine genomic sequences, a file transfer protocol can be used instead to directly transfer the file from NCBI.

In detail, a canine nucleotide sequence database is obtained from GenBank through file transfer protocol (ftp://ftp.ncbi.nih.gov). As described in Example 1, Java-based software programs are used to sequentially: 1) curate sequences specific to canis familiaris, 2) select coding sequences, 3) select high-quality, vector-trimmed regions of expressed sequence tags (ESTs), 4) convert to FASTA format, 5) prune by cluster analysis to eliminate duplication, and 6) select sequences with complete 3′ sequencing. For annotation and sense orientation confirmation, the canine ESTs are blasted against a similarly generated Human/MouseCDS using the BlastN algorithm at the Ohio SuperComputer Center facility. Sequences below the threshold E value (<10⁻⁸) are selected for further annotation. Annotated sequences are blasted against the fully annotated SwissProt protein database to further confirm annotation and sequence orientation. Table 35 lists an annotation of the canine sequences identified in accordance with the invention; Table 36 shows the canine sequences (SEQ ID NOS 3290-3797).

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

LENGTHY TABLE REFERENCED HERE
US20080233564A1-20080925-T00001
Please refer to the end of the specification for access instructions.

LENGTHY TABLE REFERENCED HERE
US20080233564A1-20080925-T00002
Please refer to the end of the specification for access instructions.

LENGTHY TABLE REFERENCED HERE
US20080233564A1-20080925-T00003
Please refer to the end of the specification for access instructions.

LENGTHY TABLE REFERENCED HERE
US20080233564A1-20080925-T00004
Please refer to the end of the specification for access instructions.

LENGTHY TABLE REFERENCED HERE
US20080233564A1-20080925-T00005
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20080233564A1). An electronic copy of the table will also be available from the USPTO upon request and payment of the fee set forth in 37 CFR 1.19(b)(3).

What is claimed is:

1. A method of preparing a species-specific nucleic acid database comprising:
selecting from a species-non-specific nucleic acid database species-specific nucleic acids comprising coding sequences;
selecting from a species-non-specific nucleic acid database species-specific nucleic acids comprising noncoding sequences;
selecting from the coding sequences those sequences that are 3′-compete or 3′-coding biased, wherein 3′-coding biased sequences comprise 5′-partial sequences having desirable characteristics;
selecting from the noncoding sequences those sequences that include poly-A tails or are derived from sequences that include poly-A tails;
reducing redundancy in selected sequences;
comparing sequences comprising unannotated sequences to a collection of sequences comprising annotated coding sequences and selecting those sequences satisfying a threshold of similarity; and
collecting all selected sequences.

2. The method according to claim 1, wherein the species-specific nucleic acid database is an equine-specific nucleic acid database.

3. The method according to claim 1, wherein the species-non-specific nucleic acid database is GenBank.

4. An array comprising a plurality of oligonucleotide probes designed to be complementary to and hybridize under stringent conditions with a gene listed in one of Tables 33, 35, or 37.

5. The array according to claim 4, wherein the array consists of less than 100 probes that are complementary to genes not listed in Tables 33, 35, or 37.

6. The array according to claim 4, wherein the array is designed for diagnosis of disease.

7. The array according to claim 6, wherein the array is designed for diagnosis of equine or canine disease.

8. The array according to claim 4, wherein the array comprises at least one gene or sequence shown in Table 9 or 10, and wherein in the array is designed for diagnosis of disease in any tissue of any animal.

9. An array comprising a plurality of oligonucleotides, wherein:
a) the oligonucleotides are chosen from the nucleic acid sequences shown in Tables 34, 36, or 38, and wherein the array comprises 10 or more of said oligonucleotides; or
b) the oligonucleotides comprise nucleotide probes designed to be complementary to, or hybridize under stringent conditions with, 10 or more nucleic acid sequences shown in Tables 34, 36, or 38.

10. The array according to claim 9, wherein the oligonucleotides comprise nucleotide probes designed to be complementary to, or hybridize under stringent conditions with, 1000 or more nucleic acid sequences shown in Table 6.

11. The array according to claim 10, wherein the oligonucleotides comprise nucleotide probes designed to be complementary to, or hybridize under stringent conditions with, 2000 or more nucleic acid sequences shown in Table 6.

12. The array according to claim 11, wherein the oligonucleotides comprise nucleotide probes designed to be complementary to, or hybridize under stringent conditions with, 3000 or more nucleic acid sequences shown in Table 6.

13. A method for populating a database of species-specific nucleic acid sequences, comprising:
querying a database of nucleic acid sequences to identify nucleic acid sequences associated with a subject species;
processing the identified sequences to create a first subset containing coding sequences and a second subset containing non-coding sequences;
dividing the first subset into a plurality of DNA sequences, if present, and a plurality of mRNA sequences;
processing the plurality of DNA sequences to derive a plurality of virtual mRNA sequences;
dividing the plurality of mRNA sequences into a plurality of complete and mRNA 3′ partial sequences, and a plurality of mRNA 5′ partial sequences;
processing the plurality of mRNA 5′ partial sequences to identify a subset of mRNA 5′ partial sequences, each member of the subset satisfying a threshold level of completeness;
identifying members of the second subset containing non-coding sequences that correlate with at least one known coding sequence of at least one species other than the subject species; and
combining the plurality of virtual mRNA sequences, the plurality of complete and mRNA 3′ partial sequences, the subset of mRNA 5′ partial sequences, and the identified correlated sequences to create the database of species-specific nucleic acid sequences.

14. The method according to claim 13, wherein the step of identifying includes comparing each member of the second subset to each member of a database containing annotated human nucleic acid sequences.

15. The method according to claim 13, wherein the step of identifying includes comparing each member of the second subset to each member of a database containing annotated human and mouse nucleic acid sequences.

16. The method according to claim 15, wherein the database containing annotated human and mouse nucleic acid sequences is derived from the database of nucleic acid sequences.

17. The method according to claim 13, further comprising eliminating duplicates within the database of species-specific nucleic acid sequences.

18. The method according to claim 13, further comprising populating the database of species-specific nucleic acid sequences with selected species-specific virus definitions.

19. The method according to claim 13, further comprising verifying that each of the identified correlated sequences is represented in sense format.

20. A method of identifying changes in gene expression with time, comprising assaying a biological sample with the microarray according to claim 4, repeating the assay after a period of time has elapsed, and comparing the results.

21. A method of detecting or monitoring a disease chosen from osteoarthritis, joint inflammation, neurological diseases, developmental orthopedic diseases, laminitis, and the general condition of stress, comprising testing a biological sample on a microarray according to claim 4 for the presence of a genetic marker associated with the disease being tested for.

22. The method according to claim 21, wherein the neurological disease is equine protozoal myelitis.

23. A method of detecting or monitoring an infectious disease chosen from herpesvirus-2 and equine protozoal myelitis caused by sarcocystis neurona or sarcocystis neurospora, comprising testing a biological sample on a microarray according to claim 4 for the presence of a genetic marker associated with the disease being tested for.
